(19) United States
(12) Patent Application Publication
(10) Pub. No.: US 2024/0108753 A1
(43) Pub. Date: Apr. 4, 2024

(54) NUCLEIC ACID DELIVERY

(71) Applicant: NUNTIUS THERAPEUTICS LIMITED, London, Greater London (GB)

(72) Inventor: Albert KWOK, London, Greater London (GB)

(21) Appl. No.: 18/274,869

(22) PCT Filed: Jan. 28, 2022
(86) PCT No.: PCT/EP2022/052145

§ 371 Date: Jul. 28, 2023

(30) Foreign Application Priority Data
Jan. 29, 2021 (GB) 2101301.6
Mar. 15, 2021 (GB) 2103551.4

(51) Int. Cl. A61K 48/00; A61K 47/64

(52) U.S. Cl. CPC A61K 48/0041; A61K 47/641; A61K 48/0058

(57) ABSTRACT

This invention provides lipid/dendrimer systems that can deliver therapeutic molecules such as nucleic acids to mammalian cells, and to the human and animal body. For instance, the invention provides systems that enable effective delivery of nucleic acids to the lymphoid organs, skeletal muscle, brain and adipose tissues, as well as to tumour tissue, liver and lungs. Delivery of DNA and RNA is provided. In particular, the systems of the invention can deliver of mRNA.

### FIELD OF THE INVENTION

The present invention relates to compositions that can deliver therapeutic molecules such as nucleic acids to mammalian cells, and to the human and animal body. Delivery to certain cell types is shown, e.g. to white blood cells in the lymphoid tissues and in circulation. Delivery to certain organs and tissues is also shown. For instance, the invention provides compositions that enable effective delivery of nucleic acids to the spleen, lymphoid organs, skeletal muscle, brain and adipose tissues, as well as to lungs, tumour tissue, heart, skeletal muscle, adipose tissue, brain, liver and kidney. Delivery of DNA and RNA is provided. In particular, delivery of mRNA is shown.

### BACKGROUND

To realise the full potential of nucleic acid therapeutics, as the next generation of precision medicines to transform healthcare, the key challenge of drug delivery must be addressed. Limitations of current viral and non-viral vector platforms are severely hampering developments, with many therapies failing at the clinical translation stage due to off-target effects, immune system activation and difficult manufacturing at scale.

In the context of in vivo delivery of nucleic acids, virus derived agents are the most potent vectors and some are very advanced in the clinics (Sheridan et al, 2011). In particular, adeno-associated viruses (AAV) are effective systems for in vivo delivery (Wang et al, 2019). The applications of such systems are, however, limited as they are only capable of transporting DNA of <5 kb and cannot transport RNA or larger DNA. In addition, despite the advancement of viral vectors, problems such as potential random insertions and immunotoxicity are associated with the use of nucleic acid delivery vectors of this kind. For example, AAV delivery can be highly immunogenic, especially when high doses are needed to target tissues other than the liver. AAV delivery systems also suffer from a limitation on repeat dosing. Patients will typically develop immunity to the AAV delivery system, making repeat dosing non-viable. Finally, manufacturing of AAV delivery systems is expensive and difficult to manufacture at scale and at Good Manufacturing Practice (GMP) grade.

Given the shortcomings of viral systems, non-viral vectors for delivering nucleic acids to cells and tissues in vivo are being investigated. Different delivery technologies have been developed for the delivery of smaller nucleic acids such as siRNA and antisense oligonucleotides (ASOs). These include bio-conjugated oligonucleotide delivery systems, wherein an siRNA or ASO is conjugated to an antibody or ligand (Benizri et al, 2019). But siRNA and ASO therapies can only achieve gene silencing or exon skipping, not gene expression. For many diseases, expressing a functional gene with DNA or mRNA would be advantageous. It is challenging to apply conjugating systems for the effective delivery of large genetic payloads such as plasmid DNA or mRNA. Plasmid DNA or mRNA are large, negatively charged molecules and cannot easily pass through the negatively charged cell membrane. It is useful when delivery vehicles encapsulate them and neutralise the charge for effective delivery.

Lipid nanoparticles (LNPs), as encapsulating non-viral delivery vehicles, have been used for mRNA delivery, for example in COVID-19 vaccines (Qui et al, 2021). To educate the immune system to fight against a virus, it is sufficient to transfect a relatively small number of immune and muscle cells locally to the site of injection. Therefore, the RNA-based COVID-19 vaccines are administered intramuscularly. However, a wide range of diseases can only be effectively treated if many cells of a specific type are transfected, which may require intravenous injection. Surface characteristics of LNPs currently used in the clinic make them a good fit for targeting the liver when administered intravenously. But the LNPs currently used in the clinic are less suitable for targeting many other tissues.

Various liposome based systems have been investigated as well, e.g. Involving uncharged lipids such as dioleoylphosphatidylethanolamine (DOPE) and/or cationic lipids such as 1,2-dioleoyl-3-trimethylammonium-propane chloride (DOTAP) and N-[1-(2,3-dioleyloxy)propyl]-N,N,N-trimethylammonium chloride (DOTMA) (Braum, 2019; Ren et al, 2000). Similar liposomes have been used in the clinic for nucleic acid delivery, showing modest efficacy.

The addition of peptides to lipid based vectors has been investigated. In peptide/lipid hybrid systems, the peptides and lipids associate with the nucleic acid to form nanoparticles that can be internalised by cells. The peptide element may be linear (Kwok et al, 2016) or branched, such as a peptide dendrimer (Kwok et al, 2013). However, this prior work on peptide/lipid hybrid systems was only performed on double-stranded plasmid DNA (pDNA) and short interfering RNA (siRNA) in serum-free in vitro conditions, differing substantially from in vivo conditions. It was not clear if this system can be used for nucleic acid delivery in vivo. Only the delivery of short single-stranded oligonucleotides using peptide dendrimer/lipid nanoparticles has been reported for in vivo (Saher et al, 2018; Saher et al, 2019), observing a low 20-30% increase in ASO liver delivery compared to ASO alone. From this data one may conclude that the peptide dendrimer system may not be able to deliver even larger nucleic acids such as mRNA to tissues in vivo. Delivery of long nucleic acids such as mRNA has never been demonstrated in vivo before with peptide dendrimer/lipid hybrid systems.

The present invention has been devised in light of the above considerations.

### SUMMARY OF THE INVENTION

The development of nucleic acid therapies is dependent on efficient nucleic acid delivery. The inventors have developed a framework for nucleic acid delivery, using peptide dendrimers. These dendrimers are branched peptides displaying one, two, three or four amino acid residues between ‘branching residues’ which act as branching units within the dendrimer molecule. This invention is surprisingly effective at delivering larger nucleic acids (e.g. larger than antisense oligonucleotides, ASOs). While some smaller nucleic acids such as ASOs are readily taken up into cells even without vector systems, the compositions of the invention allow larger nucleic acids such as mRNA to be effectively delivered. This opens up fields such as CRISPR mediated gene editing and related technologies to clinical application without needing viral delivery vectors.

Specifically developing a tissue-specific mRNA delivery system represents a significant advancement as there is still a lack of clinical mRNA or CRISPR cassette (i.e. sgRNA and Cas9 mRNA) delivery systems that bypass the liver. The present invention can effectively deliver the CRISPR cassettes in cells and mRNA to various tissues, especially to the lungs and immune cells rich tissues, including spleen, lymph nodes and bone marrow. This allows the development of mRNA and CRISPR therapy to extrahepatic tissues. The ability to deliver mRNA to immune cells, to a greater degree the myeloid cells including monocytes, macrophages, neutrophils and dendritic cells, enables this technology to be developed to treat all cancer types (including solid tumours and blood cancer such as Myelodysplastic Syndromes (MDS) and Chronic Myelomonocytic Leukemia (CMML), autoimmune diseases (e.g. Rheumatoid Arthritis, Crohn's diseases, Uveitis, Inflammatory bowel disease) and other immune cell related disorders such as Gaucher's diseases. This invention also allows mRNA to be delivered to the lungs and/or immune cells, making treatments for lung-related diseases such as cystic fibrosis and chronic obstructive pulmonary diseases possible.

The inventor has also found that their dendrimer based system is surprisingly versatile. DNA and RNA molecules can be delivered using a range of first generation, second generation and third generation dendrimers. In one example, a vector system based on dendrimers such as the G1,2,3-KL dendrimer (a ‘third generation’ dendrimer) associated with lipids can improve cell transfection of DNA by 6-10 fold compared to some leading commercial reagents such as Lipofectamine 2000. In another example, a vector system based on dendrimers such as the G1,2-RL,3-LR dendrimer (a ‘third generation’ dendrimer) associated with lipids can improve cell transfection nucleic acids such as mRNA by 10 fold compared to some leading commercial reagents such as Lipofectamine 2000. Moreover, certain organs and tissues can be targeted. As described in more detail below, the inventors have found dendrimer based systems to be surprisingly robust and versatile, exhibiting high activity in the presence of serum in vitro and exhibiting unexpected tissue targeting characteristics in vivo. Effective delivery of DNA and RNA are discussed below.

The inventors have found that, for DNA delivery, G1,2,3-KL and G1,2,3-RL (‘third generation’ dendrimers) and to some degree G1,2-KL, G1,2-RL (both ‘second-generation’ dendrimers) are able to transfect HeLa and Neuro2A cells in the presence of serum. (Serum components present a challenge for in vivo DNA delivery, especially systemic delivery, because components such as albumin can interfere with cationic formulations.) This unexpected finding led the inventors to investigate the mechanism underlying cell entry, and to assess the capacity of this vector system in vivo. Surprisingly, the G1,2,3-RL based vector was found to mediate effective delivery of functional nucleic acids into certain tissues following systemic delivery. Liver and skeletal muscles are highly targeted. Endocytic pathway analysis indicates that the G1,2,3-RL DNA complexes delivers DNA via both clathrin, caveolae mediated endocytosis and macropinocytosis.

The inventors have found that, for mRNA delivery, dendrimers with 1 or 2 generations (e.g. RHCG1-R, RHCG1,2-R, RHCG1-LR, RHCG1-RL, 2-LR) can transfect cells effectively and mediate similar transfection efficiency as dendrimers with 3 generations (see FIG. 28A, which shows the efficacy of transfecting HeLa cells under full growth media conditions, including serum). This indicates particular versatility of the hybrid system for RNA delivery.

The dendrimers used in the invention are first, second or third generation peptide dendrimers, meaning that they have up to three ‘layers’ of peptide motifs (which are typically dipeptide motifs) interspersed between ‘branching’ residues, such as lysine. First generation dendrimers have the following structure, shown in the N-termini to C-terminus orientation, and taking Lys to be the branching unit:
(N-term-Pep1)₂-Lys-(Core)-(C-term)

Second generation dendrimers have the following structure, shown in the N-termini to C-terminus orientation, and taking Lys to be the branching unit:
(N-term-Pep2)₄-Lys₂(Pep1)₂-Lys-(Core)-(C-term)

Third generation dendrimers have the following structure, shown in the N-termini to C-terminus orientation, and taking Lys to be the branching unit:
(N-term-Pep3)₈-Lys₄-(Pep2)₄-Lys₂-(Pep1)₂-Lys-(Core)-(C-term)

Third generation dendrimers are represented diagrammatically (with N-termini on the left and C-terminus on the right) in FIG. 31.

The circle represents the core sequence. Each triangle represents a branching residue, such as lysine. Each rectangle represents a peptide motif. There are two peptide motifs in the first layer, four peptide motifs in the second layer, and eight peptide motifs in the third layer of the third generation dendrimer. The N- and C-termini may be derivatised with further chemical motifs, as discussed herein. For instance, while in underivatized embodiments, the C-terminus is a carboxylic acid, in other embodiments the C-terminus is derivatised e.g. to comprise a primary amide group, CONH₂ (instead of COOH), as a result of the chemical pathway used to synthesise the dendrimer. Functionally important derivatizations such as targeting moieties (e.g. antibodies, peptide groups, sugar groups and/or lipid chains) are also envisaged, which can be attached to the N- and/or C-termini, or at other positions along the dendrimer.

As described herein, the dendrimers can be first, second or third generation. This can be defined structurally as follows: First generation dendrimers comprise a core peptide sequence, a first branching residue and two first peptide motifs. The two first peptide motifs independently consist of a single amino acid, dipeptide, tripeptide or tetrapeptide motifs. Second generation dendrimers further comprise two second branching residues (e.g. lysine) and four second peptide motifs, wherein one of the second branching residues is covalently bound to one of the first peptide motifs and the other second branching residue is covalently bound to the other first peptide motif, and wherein each second branching residue is covalently bound to two second peptide motifs. The four second peptide motifs independently consist of a single amino acid, dipeptide, tripeptide or tetrapeptide motifs. Third generation dendrimers further comprises four third branching residues (e.g. lysine) and eight third peptide motifs, wherein each second peptide motif is respectively covalently bound to one of the third branching residues such that each third branching residue is covalently bound to one second peptide motif, and wherein each third branching residue is covalently bound to two third peptide motifs. The eight third peptide motifs independently consist of a single amino acid, dipeptide, tripeptide or tetrapeptide motifs. Each of the first, second and third peptide motifs, where present, may comprise (1) an amino acid with a basic side chain such as, but not limited to, Lysine (K) or Arginine (R) or Histidine (H), (2) an amino acid with an acidic side chain such as but not limiting to Aspartic acid (D) and Glutamic acid (E), (3) an amino acid with a non-polar side chain such as, but not limited, to Glycine (G), Alanine (A), Valine (V), Isoleucine (I), Leucine (L), Methionine (M), Phenylalanine (F), Beta-alanine (B), Tryptophan (W), Proline (P), aminohexanoic acid (X) and Cysteine (C) and (4) an amino acid with a uncharged polar side chain such as, but not limited to, Asparagine (N), Glutamine (Q), Serine (S), Threonine (T) and Tyrosine (Y).

Preferred dendrimers are presented in Table 2 below. Certain examples are discussed in particular. For instance, in dendrimers where each peptide motif is an Arg-Leu (RL) dipeptide, this structure can be denoted G1-RL, G1,2-RL and G1,2,3-RL. In dendrimers where each peptide motif is a Lys-Leu (KL) dipeptide, this structure is denoted G1-KL, G1,2-KL and G1,2,3-KL. In dendrimers where each peptide motif is a Leu-Arg (LR) dipeptide, this structure is denoted G1-LR, G1,2-LR and G1,2,3-LR.

(‘G1’. ‘G2’ and ‘G3’ refer to the ‘generation-1’, ‘generation-2’ and ‘generation-3’ peptide motifs of the first, second and third layers, respectively. Each amino acid residue can be an L-amino acid or a D-amino acid. D-amino acids may be designated using lower case letters in the single-letter code. Alternatively, dendrimers in which each amino acid is the D-isoform can be written with a preceding “D-” before the short-form denotation of the dendrimer)

Thus, in a first aspect, this invention provides a composition for use in medicine, wherein the composition comprises a first, second or third generation peptide dendrimer, a nucleic acid and a lipid. The composition further comprises a nucleic acid and a lipid. The peptide dendrimer comprises at least: a core peptide sequence, a first branching residue and two first peptide motifs. The branching residue may be lysine, 2,4-diaminobutyric acid, omithine, or diaminopropionic acid. The nucleic acid comprises a nucleic acid of at least 30, at least 35, at least 40, at least 45, at least 50, at least 55, at least 60, at least 65, at least 70, at least 75, at least 80, at least 85, at least 90, at least 95, or at least 100 nucleotides.

In a second aspect, this invention provides a composition comprising a peptide dendrimer, a nucleic acid and a lipid, wherein the peptide dendrimer comprises at least: a core peptide sequence, a first branching residue and two first peptide motifs. The branching residue may be lysine, 2,4-diaminobutyric acid, omithine, or diaminopropionic acid. The nucleic acid comprises a single-stranded nucleic acid of at least 30, at least 35, at least 40, at least 45, or at least 50, at least 55, at least 60, at least 65, at least 70, at least 75, at least 80, at least 85, at least 90, at least 95, or at least 100 nucleotides.

In a third aspect, this invention provides a method of delivering a nucleic acid into a cell in a subject in need of the delivery, comprising administering a pharmaceutically effective amount of a composition to the subject. The composition comprises a peptide dendrimer, a nucleic acid and a lipid, wherein the peptide dendrimer comprises at least: a core peptide sequence, a first branching residue and two first peptide motifs. The branching residue may be lysine, 2,4-diaminobutyric acid, omithine, or diaminopropionic acid. The nucleic acid comprises a nucleic acid of at least 30, at least 35, at least 40, at least 45, at least 50, at least 55, at least 60, at least 65, at least 70, at least 75, at least 80, at least 85, at least 90, at least 95, or at least 100 nucleotides. The nucleic acid may be single stranded.

In some embodiments, the nucleic acid is RNA. For instance, the RNA may be selected from an mRNA, an ssRNA, a dsRNA, an sgRNA, a crRNA, a tracrRNA, a lncRNA, an siRNA, an saRNA and/or a self-amplifying RNA. Preferably, the RNA is mRNA.

In some embodiments, the nucleic acid is DNA. For instance, the DNA may comprise a ssDNA, a dsDNA, a plasmid, and/or a cDNA.

To avoid any doubt; the composition may comprise more than one nucleic acid (for instance more than one type of RNA molecule). Similarly, the composition may comprise more than one lipid. The composition may comprise more than one peptide dendrimer.

In some embodiments, the composition comprises an RNA nucleic acid and a DNA nucleic acid. The RNA nucleic acid and a DNA nucleic acid may be part of a single nucleic acid molecule.

In some embodiments, the nucleic acid comprises a modified nucleic acid. Exemplary nucleic acid modifications are described herein.

Preferably, the nucleic acid encodes a transgene and can express the transgene in a target cell. The transgene may be a protein or peptide. Additionally or alternatively, the nucleic acid can modulate expression or activity of an endogenous gene. The modulation can be an increase in the expression of the gene and/or exogenous expression of further copies of the gene, or the modulation can be a decrease in the expression of the gene.

In some embodiments the modulated endogenous gene is a gene that expresses a protein or peptide.

In some embodiments, the protein or peptide comprises an antigen, a hormone, a receptor, a chimeric antigen receptor, a transcription factor and/or a cytokine.

In some embodiments, the transgene comprises a tumour antigen, a viral protein, a bacterial protein or a protein of a microorganism that is parasitic to a mammal.

In some embodiments the composition can be used as a vaccine.

In some embodiments, the nucleic acid comprises or encodes a self-amplifying RNA.

In some embodiments, the use comprises a treatment for a genetic disorder in the subject.

In some embodiments, the nucleic acid expresses a functional version of a gene that is non-functional, downregulated, inactive or impaired in the subject.

In some embodiments, the nucleic acid encodes and/or comprises one or more components of a system for editing a genome or a system for altering gene expression. For instance, the system for editing a genome or a system for altering gene expression may be a CRISPR/Cas system. The nucleic acid may encode a Cas protein or peptide, and/or comprises an sgRNA, a crRNA, and/or a tracrRNA. The nucleic acid may comprise an mRNA encoding a Cas protein or peptide, and an RNA sequence comprising sgRNA. The composition may comprise an mRNA that encodes a Cas protein or peptide, and another RNA comprising sgRNA (as separate molecules). In some embodiments, one or more of the sgRNA, crRNA, tracrRNA and nucleic acid encoding a Cas protein, where present, are part of a single nucleic acid. In some embodiments, one or more of the sgRNA, crRNA, tracrRNA and nucleic acid encoding a Cas protein, where present, are present on two or more nucleic acids.

In some embodiments, the composition is targeted to spleen, lymph tissue, skeletal muscle, brain and adipose tissues, as well as to lungs, tumour tissue, heart, skeletal muscle, adipose tissue, brain, liver and kidney.

In some embodiments, the composition is targeted to spleen, lymph tissue, lung and/or bone. In these embodiments, the nucleic acid may be RNA, e.g. mRNA.

In some embodiments, the nucleic acid is delivered to a cell that is a leucocyte, e.g. a B lymphocyte, a T lymphocyte, a monocyte, a neutrophil, a dendritic cell, a macrophage, or a monocyte; a lymph node tissue cell, a myeloid cell, a fibroblast, a myocyte, a skeletal myocyte, an endothelial cell, a hepatocyte, a stellate cell, a neuron, an astrocyte, a splenocyte, a lung cell, a cardiomyocyte, a kidney cell, an adipose cell, a stem cell and/or a tumour cell.

In some embodiments, the composition is administered to a subject such that the nucleic acid is delivered to an immune cell.

In some embodiments, the nucleic acid expresses an immune molecule or a transcription factor in the target cell. The immune molecule may be a T cell receptor, chimeric antigen receptor, a cytokine, a decoy receptor, an antibody, a costimulatory receptor, a costimulatory ligand, a checkpoint inhibitor, an immunoconjugate, or a tumour antigen.

In some embodiments, the cell is a B lymphocyte, a T lymphocyte, a monocyte, a neutrophil, a dendritic cell, a macrophage, a monocyte, myeloid derived suppressor cell (MDSC), a tumour associated macrophage or a tumour associated neutrophil.

In some embodiments the nucleic acid is RNA, e.g. mRNA.

In some embodiments, the composition is for use in a method of treating cancer in the subject. The cancer may be a blood cancer, for example leukaemia, lymphoma, myeloma, or myelodysplastic syndrome; or a lung cancer, a cardiac cancer, a sarcoma, or liver cancer. The treatment may also comprise administration of an anticancer agent. The cancer may be Non-small cell lung cancers, Advanced melanoma, Prostate cancer, Ovarian cancer, Breast cancer, Lung cancer, Bile duct cancer (Cholangiocarcinoma), Gallbladder cancer, Neuroendocrine tumors, Hepatocellular carcinoma, Colorectal cancer, Pancreatic cancer and Solid tumors.

In some embodiments, the composition is for use in a method of treating a lung disease. For instance, the composition may be for use in treating chronic obstructive pulmonary disease (COPD) or cystic fibrosis (CF).

In some embodiments, the composition is for use in a method of treating an autoimmune disease in the subject.

In some embodiments, the composition is for use in a treatment for Pompe disease, a muscle wasting disease, a myopathy, or a muscular dystrophy, e.g. Duchenne muscular dystrophy in the subject. In these embodiments, the nucleic acid may be DNA.

In some embodiments, the composition is for use in a treatment for a limb ischemia, such as diabetic limb ischemia, in the subject, and wherein the transgene is hepatocyte growth factor (HGF), vascular endothelial growth factor (VEGF) and/or Fibroblast growth factor (FGF).

In some embodiments, the two first peptide motifs independently consist of a single amino acid, dipeptide, tripeptide or tetrapeptide motifs.

In some embodiments, the peptide dendrimer further comprises two second branching residues (e.g. lysine) and four second peptide motifs, wherein one of the second branching residues is covalently bound to one of the first peptide motifs and the other second branching residue is covalently bound to the other first peptide motif, and wherein each second branching residue is covalently bound to two second peptide motifs.

In some embodiments, the four second peptide motifs independently consist of a single amino acid, dipeptide, tripeptide or tetrapeptide motifs.

In some embodiments, the peptide dendrimer further comprises four third branching residues (e.g. lysine) and eight third peptide motifs, wherein each second peptide motif is respectively covalently bound to one of the third branching residues such that each third branching residue is covalently bound to one second peptide motif, and wherein each third branching residue is covalently bound to two third peptide motifs.

In some embodiments, the eight third peptide motifs independently consist of a single amino acid, dipeptide, tripeptide or tetrapeptide motifs.

Each peptide motif independently comprises naturally occurring L-or D-amino acids and/or non-naturally occurring L-or D-amino acids, for example, Beta-alanine (B) or aminohexanoic acid (X).

In some embodiments, the first, second and/or third peptide motifs comprise an amino acid with a basic side chain.

In some embodiments, the core sequence comprises an amino acid residue with an ionisable group such as histidine.

In some embodiments, the first, second and/or third peptide motifs comprise an amino acid with a non-polar side chain.

In some embodiments, the first, second and/or third peptide motifs comprise an amino acid with an acidic side chain.

In some embodiments, the first, second and/or third peptide motifs comprise an amino acid with an uncharged polar side chain.

In some embodiments, the first, second and third peptide motifs (where present) comprise a) an arginine (R) or lysine (K); and/or b) a leucine (L), valine (V), histidine (H) or isoleucine (I).

In some embodiments, the first, second and/or third peptide motifs comprise a leucine (L) and/or arginine (R) residue.

In some embodiments, the peptide dendrimer comprises a structure set forth in Table 2.

In some embodiments, the peptide dendrimer further comprises a tissue and/or cell targeting motif. The tissue or cell targeting motif may comprise a muscle targeting motif, for example, GAASSLNIA (SEQ ID NO: 1), an integrin targeting motif, for example arginine-glycine-aspartic acid (RGD) or a chemical modification, for example comprising mannose glycosylation.

In some embodiments, the peptide dendrimer further comprises a cell penetrating peptide. The cell penetrating peptide may comprises a TAT derived sequence. The cell penetrating peptide may comprise the peptide sequence XRXRRBRRXRRBRXB (SEQ ID NO: 2), where X is 6-aminohexanoic acid and B is beta-alanine.

In some embodiments, the peptide dendrimer further comprises an alkyl chain, alkenyl chain, an antibody or a fragment thereof, a sugar, and/or a fatty acid. An alkyl or alkenyl chain may be conjugated to the core peptide sequence, for instance at the C terminus of the peptide dendrimer. Alternatively, the alkyl or alkenyl chain may be conjugated to the N terminus of the peptide dendrimer.

In some embodiments, alkyl or alkenyl chains comprise from about 5 carbons to about 50 carbons, preferably from about 12 to about 30 carbons.

In some embodiments, the peptide dendrimer comprises a fatty acid conjugated to the C terminus of the peptide dendrimer. In other embodiments, the peptide dendrimer comprises a fatty acid conjugated to the N terminus of the peptide dendrimer.

In some embodiments, the lipid of the composition comprises a cationic lipid, a neutral lipid, an anionic lipid and/or an ionisable lipid.

In some embodiments, the lipid of the composition comprises a saturated fatty acid. Additionally or alternatively, the lipid of the composition may comprise an unsaturated fatty acid.

In some embodiments, the lipid comprises 1, 2, 3, 4, 5 or 6 fatty acid chains. Preferably, the lipid comprises 2, 3, 4 or 6 fatty acid chains.

In some embodiments, the lipid comprises dioleoylphosphatidylethanolamine (DOPE) and/or N-[1-(2,3-dioleyloxy)propyl)-N,N,N-trimethylammonium chloride (DOTMA). In some embodiments, the lipid comprises dioleoylphosphatidylethanolamine (DOPE) and dioleoylphosphatidylglycerol (DOPG).

In some embodiments, the N/P ratio is between about 0.01:1 and 100:1. For instance, the N/P ratio may be between about 0.05: and 50:1, or between about 0.1 and 30:1. Narrower ranges such as between 0.2: and 25:1, and between 0.5:1 and 20:1 are also envisaged.

In some embodiments, the N/P ratio is between 1:1 and 50:1. In some embodiments, a higher proportion of the composition is observed in the spleen and/or lymph nodes than the liver following administration to a subject.

In some embodiments, the N/P ratio is between 0.01:1 and 1:1. In some embodiments, a higher proportion of the composition is observed in the lung, spleen and/or lymph nodes than the liver following administration to a subject.

In some embodiments, the peptide dendrimer, nucleic acid and lipid form a positively charged particle.

In other embodiments, the peptide dendrimer, nucleic acid and lipid form a negatively charged particle or a particle with neutral charge.

In some embodiments, delivery of the nucleic acid to the target tissue or target cell is increased by at least 10%, 15%, 20%, 25%, 30%, 35%, 40%, 45%, 50%, 60%, 85%, 75%, 85%, 90%, 95%, 100% compared to delivery of the same nucleic acid to the same tissue or cell type using a lipid based nucleic acid delivery system. The target cell or target tissue is a cell type or organ/tissue defined herein. For instance, the target tissue may be spleen, lymphoid organs, skeletal muscle, brain and adipose tissues, as well as to lungs, tumour tissue, heart, skeletal muscle, adipose tissue, brain, liver and kidney. The lipid based nucleic acid delivery system may be DOTMA/DOPE.

The composition can be administered to a subject intravenously, intramuscularly, Intratumorally, subcutaneously, intradermally or intraperitoneally.

In some embodiments, the composition is comprised within a liquid. In other embodiments, the composition is provided as a dry composition, e.g. a dry powder. The dry composition may be prepared using lyophilisation and/or freeze-drying techniques.

In a particular embodiment; a third generation peptide dendrimer comprises: a first lysine residue and two first peptide motifs; two second lysine residues and four second peptide motifs; four third lysine residues and eight third peptide motifs; and a core peptide sequence which is covalently bound to the first lysine residue,
(i) wherein the first lysine residue is covalently bound to two first peptide motifs, which are respectively covalently bound to the two second lysine residues;
(ii) wherein each second lysine residue is covalently bound to two second peptide motifs, wherein each second peptide motif is respectively covalently bound to one of the third lysine residues; and
(iii) wherein each third lysine residue is covalently bound to two of the third peptide motifs, wherein the first, second and third peptide motifs are independently monopeptide, dipeptide, tripeptide or tetrapeptide motifs. Each of the first, second and third peptide motifs may comprise an arginine (R) or lysine (K); and/or b) a leucine (L), valine (V), histidine (H) or isoleucine (I). (The term “covalently bound” means a direct covalent bond between the recited moieties, without any intervening atoms. Thus, only the mono-, di-, tri- or tetra-peptide motif subsists between the first lysine residue and each second lysine residue; and only the mono-di-, tri-, or tetra-peptide motifs subsist between each second lysine residue and each third lysine residue.)

In some embodiments, each of the first, second and third peptide motifs, where present, may comprise (1) an amino acid with a basic side chain such as, but not limited to, Lysine (K) or Arginine (R) or Histidine (H), (2) an amino acid with an acidic side chain such as but not limiting to Aspartic acid (D) and Glutamic acid (E), (3) an amino acid with a non-polar side chain such as, but not limited, to Glycine (G), Alanine (A), Valine (V), Isoleucine (I), Leucine (L), Methionine (M), Phenylalanine (F), Beta-alanine (B), Tryptophan (W), Proline (P), aminohexanoic acid (X) and Cysteine (C) and (4) an amino acid with a uncharged polar side chain such as, but not limited to, Asparagine (N), Glutamine (Q), Serine (S), Threonine (T) and Tyrosine (Y).

Preferably at least one of the first, second and third peptide motifs comprise an arginine (R). At least two of the first, second and third peptide motifs may comprise an arginine (R). In some embodiments, all of the first, second and third peptide motifs comprise an arginine (R).

Preferably at least one of the first, second and third peptide motifs comprise a leucine (L). At least two of the first, second and third peptide motifs may comprise a leucine (L). In some embodiments, all of the first, second and third peptide motifs comprise a leucine (L).

In some embodiments, each of the first, second and third peptide motifs are dipeptide motifs. In some embodiments, each of the first, second and third peptide motifs are tripeptide motifs. In some embodiments, each of the first, second and third peptide motifs are tetrapeptide motifs. In some embodiments, each of the first, second and third peptide motifs are independently mono-, dipeptide, tripeptide or tetrapeptide motifs. Preferably, at least one of the first, second and third peptide motifs is a dipeptide motif comprising both a leucine (L) and an arginine (R). In some embodiments, at least two of the first, second and third peptide motifs are dipeptide motifs comprising both a leucine (L) and an arginine (R). In particularly preferred embodiments, each peptide motif is a dipeptide motif comprising both a leucine (L) and an arginine (R). Each amino acid residue is independently selected from the L-isoform or D-isoform.

In some embodiments, the peptide dendrimer is selected from any one of the peptide dendrimers listed in Table 2.

In some embodiments, the peptide dendrimer is G1,2,3-RL, comprising (RL)₈(KRL)₄(KRL)₂K-core.

In some embodiments, the peptide dendrimer is G1-LR,G2,3-RL, comprising (RL)₈(KRL)₄(KLR)₂K-core.

In some embodiments, the peptide dendrimer is G1,2,3-rl, comprising (rl)₈(krl)₄(krl)₂K-core (where ‘r’ is D-arginine, ‘l’ is D-leucine and ‘k’ is D-lysine.

In some embodiments, the peptide dendrimer is G1,2-RL,G3-LR, comprising (LR)₈(KRL)₄(KRL)₂K-core.

In some embodiments, the peptide dendrimer is G1,2-LR,G3-RL, comprising (RL)₈(KLR)₄(KLR)₂K-core.

In some embodiments, the peptide dendrimer is G1,2,3-LR, comprising (LR)₈(KLR)₄(KLR)₂K-core.

In some embodiments, the peptide dendrimer is RHCG1-R, comprising (R)2KRHC-NH2.

In some embodiments, the peptide dendrimer is RHCG1,2-R, comprising (R)4(KR)2KRHC-NH2.

In some embodiments, the peptide dendrimer is RHCG1-LR, comprising (LR)2KRHC-NH2.

In some embodiments, the peptide dendrimer is RHCG1-RL, 2-LR, comprising (LR)4(KRL)2KRHC-NH2.

In some embodiments, the peptide dendrimer is G1,2-RL, 3-LR, comprising (LR)₈(KRL)₄(KRL)₂KGSC-NH₂.

In some embodiments, the peptide dendrimer is RHCG1-RLR, comprising (RLR)2KRHC-NH2.

In some embodiments, the peptide dendrimer is RHCG1,2-RLR, comprising (RLR)4(KRLR)2KRHC-NH2 In some embodiments, the peptide dendrimer is G1-LRLR, comprising (LRLR)2KGSC-NH2.

In some embodiments, the peptide dendrimer is RHCG1,2-RL, 3-LR, comprising (LR)8(KRL)4(KRL)2KRHC-NH2.

In some embodiments, the peptide dendrimer is G1,2,3-RL, comprising (RL)₈(KRL)₄(KRL)₂KGSC-NH₂.

In some embodiments, the peptide dendrimer is NTX1, comprising (LR)8(KRL)4(KRL)2KGSCGAASSLNIAXRXRRBRRXRRBRXB-NH2 (where X=6-aminohexanoic acid and B=beta-alanlne).

(‘G1’ refers to the ‘generation-1’ peptide motif of the first layer. ‘G2’ refers to the ‘generation-2’ peptide motif of the second layer. ‘G3’ refers to the ‘generation-3’ peptide motif of the third layer.)

Thus, the invention provides compositions comprising: a nucleic acid, a lipid, and a dendrimer described herein, for use in medicine. The dendrimer may be a first generation dendrimer, or a second generation dendrimer, or a third generation dendrimer. Preferably, the N/P ratio, which is the amount of peptide (measured by the number of 1+ charged nitrogen atoms on the peptide, N) to the amount of nucleic acid (measured by the number of 1− charged phosphate groups in the backbone, P) Is greater than 0.05:1, for instance greater than 0.1:1. (The N/P ratio terminology can be expressed as “N/P”, “N:P”, or “NP”.) In some embodiments the N/P ratio is 0.15:1, or about 0.15:1, or at least 0.15:1. In some embodiments the N/P ratio is 0.16:1, or about 0.16:1, or at least 0.16:1. In some embodiments, the N/P ratio is at least, or greater than, 1:1, for instance about 2:1 or greater, about 2.5:1 or greater, about 3:1 or greater, about 4:1 or greater, about 5:1 or greater, about 10:1 or up to 20:1. In some embodiments, the N/P ratio is about 5:1, about 8:1, about 10:1, or about 20:1. In some embodiments, the N/P ratio is in the range of about 2:1 to about 20:1, or about 2.5:1 to about 10:1.

The lipid component of the composition may comprise DOTMA, DOPE, DOPC and/or DOPG.

The amount of lipid component can be expressed in a weight:weight ratio (“w/w”, or “w:w”), with respect to the amount of the nucleic acid in the composition may be in the range of 1:50 to 50:1. More preferably, the amount of lipid (by weight) is 1:1 to 50:1, or 2:1 to 20:1 with respect to the amount of nucleic acid (by weight). The lipid:nucleic acid ratio can be at least 2:1. Most preferably, the weight:weight ratio of lipid:nucleic acid is about 10:1. These ratios refer to the weight of the total lipid. As described herein, the composition may comprise a lipid that includes more than one lipid component, e.g. a mixture of two, three or four lipids. The weight of the lipid component is the total (combined) weight of these lipid components. Preferably, each lipid component is mixed in approximately equal proportions.

Relatedly, the invention provides a pharmaceutical composition comprising a nucleic acid, a lipid, and a first, second or third generation dendrimer described herein, and a pharmaceutically acceptable excipient.

The nucleic acid that forms part of the compositions of the invention may be an antisense oligonucleotide (ASO). Preferably, the ASO is at least 20 nucleotides in length, at least 25 nucleotides in length, at least 30 nucleotides in length, at least 35 nucleotides in length, or has a length as specified in connection with the nucleic acids disclosed elsewhere herein, e.g. at least 40 nucleotides in length.

The nucleic acid that forms part of the compositions of the invention may be an mRNA molecule. The nucleic acid that forms part of the compositions of the invention may be a lncRNA molecule. The nucleic acid that forms part of the compositions of the invention may comprise a CRISPR sequence. The nucleic acid that forms part of the compositions of the invention may comprise a double stranded region. The nucleic acid may be an siRNA molecule. The nucleic acid may be a small activating RNA (saRNA) molecule. The nucleic acid may be a self-amplifying RNA molecule. The nucleic acid may be a DNA plasmid can express an siRNA or saRNA molecule in a target cell. The nucleic acid may be a DNA plasmid (linearized or circular), e.g. a plasmid that can express a transgene in a target cell.

The transgene may be a viral protein, a bacterial protein or a protein of a microorganism that is parasitic to a mammal. The composition expressing a viral protein, a bacterial protein or a parasitic microbial protein may be used as a vaccine. For instance, an effective amount of the composition may be delivered systemically to a subject (e.g. intravenously) to achieve expression of the viral protein, bacterial protein or parasitic microbial protein in the skeletal muscle of the subject in order to prime an immune response to that viral, bacterial or parasitic protein. Thus, this invention provides methods of vaccinating a subject, and compositions for use in the vaccination of a subject. In such examples, the transgene may be expressed in an immune cell described herein, e.g. a leucocyte, such as a B lymphocyte, a T lymphocyte, a monocyte, a neutrophil, a dendritic cell, a macrophage, or a monocyte; a lymph node tissue cell.

The transgene may express a therapeutic protein for use in a gene therapy. The gene therapy may be for treating a genetic disorder in a patient. The genetic disorder may be monogenic disorder, e.g. muscular dystrophy in the patient. In embodiments where the monogenic disorder is a muscular dystrophy, the transgene may be dystrophin. In embodiments where the disorder is ischemia, the transgene may be hepatocyte growth factor (HGF), vascular endothelial growth factor (VEGF) and Fibroblast growth factors (FGF). In embodiments where the disorder is muscle wasting, the transgene may be follistatin. In embodiments where the disorder is a neuromuscular disease, the transgene may be acid α-glucosidase (GAA). While the transgene may be expressed in one or more of the tissues disclosed herein, the expressed protein may be secreted from the tissue(s) into the circulation.

It is envisaged that this invention can be used to deliver nucleic acid therapies to treat myopathies, it is also envisaged that this invention can be used to deliver nucleic acid therapies to treat muscular dystrophies such as Duchenne muscular dystrophy, myotonic dystrophy, facioscapulohumeral muscular dystrophy, Becker muscular dystrophy, limb-girdle muscular dystrophy, oculopharyngeal muscular dystrophy, Emery-Dreifuss muscular dystrophy, inheriting muscular dystrophy, congenital muscular dystrophy, and distal muscular dystrophy.

The nucleic acid therapy may be for treating muscle wasting conditions such as cachexia. The nucleic acid therapy may be for treating other muscular disorders, such as inherited muscular disorders, e.g. myotonia congenita, or familial periodic paralysis. The nucleic acid therapy may be for treating a motor neuron disease, such as ALS (amyotrophic lateral sclerosis), spinal-bulbar muscular atrophy (SBMA) or spinal muscular atrophy (SMA). The nucleic acid therapy may be for treating a mitochondrial disease, such as Friedreich's ataxia (FA), or a mitochondrial myopathy such as Kearns-Sayre syndrome (KSS), Leigh syndrome (subacute necrotizing encephalomyopathy), mitochondrial DNA depletion syndromes, mitochondrial encephalomyopathy, lactic acidosis and stroke-like episodes (MELAS), mitochondrial neurogastrointestinal encephalomyopathy (MNGIE), myoclonus epilepsy with ragged red fibers (MERRF), neuropathy, ataxia and retinitis pigmentosa (NARP), Pearson syndrome or progressive external opthalmoplegia (PEO). The nucleic acid therapy may be for treating a congenital myopathy, such as a cap myopathy, a centronuclear myopathy, a congenital myopathies with fiber type disproportion, a core myopathy, a central core disease, a multiminicore myopathies, a myosin storage myopathies, a myotubular myopathy, or a nemaline myopathy. The nucleic acid therapy may be for treating a distal myopathy, such as GNE myopathy/Nonaka myopathy/hereditary inclusion-body myopathy (HIBM), Laing distal myopathy, Markesbery-Griggs late-onset distal myopathy, Miyoshi myopathy, Udd myopathy/tibial muscular dystrophy, VCP Myopathy/IBMPFD, vocal cord and pharyngeal distal myopathy, or Welander distal myopathy. The nucleic acid therapy may be for treating an endocrine myopathy, such as hyperthyroid myopathy or hypothyroid myopathy. The nucleic acid therapy may be for treating an inflammatory myopathy such as dermatomyositis, inclusion body myositis, or polymyositis. The nucleic acid therapy may be for treating a metabolic myopathy, such as Acid maltase deficiency (AMD, Pompe disease), camitine deficiency, camitine palmitoyltransferase deficiency, debrancher enzyme deficiency (Cor disease, Forbes disease), lactate dehydrogenase deficiency, myoadenylate deaminase deficiency, phosphofructokinase deficiency (Tarui disease), phosphoglycerate kinase deficiency, phosphoglycerate mutase deficiency, or phosphorylase deficiency (McArdle disease). The nucleic acid therapy may be for treating a myofibrillar myopathy, or a scapuloperoneal myopathy. The nucleic acid therapy may be for treating a neuromuscular Junction disease, such as congenital myasthenic syndromes (CMS), Lambert-Eaton myasthenic syndrome (LEMS), or myasthenia gravis (MG). The nucleic acid therapy may be for treating a peripheral nerve disease, such as Charcot-Marie-Tooth disease (CMT), or giant axonal neuropathy (GAN). The nucleic acid therapy may be for treating a cardiovascular disease such as Thromboangiitis obliterans/Buerger disease, diabetic peripheral neuropathy (also tested in ALS, critical limb ischemia and foot ulcers), peripheral artery disease, limb ischemia, critical limb ischemia (also known as chronic limb threatening ischemia and diabetic limb ischemia), severe peripheral artery occlusive disease (PAOD), or intermittent claudication/arteriosclerosis. The nucleic acid therapy may be for treating an infectious disease, such as COVID-19, HIV, HBV, HCV, Ebola and Marburg virus, West Nile fever, SARS, avian flu, HPV, cytomegalovirus, or malaria. The nucleic acid therapy may be for treating a cancer, such as a sarcoma, melanoma, breast cancer, lung cancer, pancreatic cancer, prostate cancer, liver cancer, acute myeloid leukaemia or B-cell lymphoma. The nucleic acid therapy may be for treating an allergy, such as peanut allergy. The nucleic acid therapy may be for treating multiple sclerosis (MS). The nucleic acid therapy may be for treating myelodysplastic syndrome (MDS).

Pompe disease results from a defect in human acid α-glucosidase (GAA), a lysosomal enzyme that cleaves terminal α1-4 and α1-6 glucose from glycogen. The composition of the invention may be used to treat Pompe disease. The composition of the invention, comprising a nucleic acid that encodes GAA, may be administered to a subject that suffers from Pompe disease in order to deliver the nucleic acid to target tissue of the subject, to express the GAA in a target tissue described herein, particularly the liver and skeletal muscle. The enzyme may be secreted from tissues into the circulation.

Follistatin is an inhibitor of TGF-β superfamily ligands that repress skeletal muscle growth and promote muscle wasting. The composition of the invention, comprising a nucleic acid that encodes follistatin, may be administered to a subject that suffers from a muscle wasting disorder in order to deliver the nucleic acid to target tissue of the subject, to express the follistatin in a target tissue described herein, particularly the liver and skeletal muscle. The protein may be secreted from tissues into the circulation.

Thus, this invention provides methods for treating such disorders, and compositions for use in such treatments.

The core peptide motif of the dendrimer is a single amino acid residue or a short peptide motif such as a dipeptide or tripeptide motif. The core sequence may comprise any amino acid (L- and/or D-Isomers), e.g. a glycine (G), a serine (S), a cysteine (C), an alanine (A), a lysine (K), a leucine (L), a valine (V), an isoleucine (I), a phenylalanine (F), a methionine (M), a tyrosine (Y), a tryptophan (W), a proline (P), a threonine (T), an asparagine (N), a glutamine (Q), an aspartic acid (D), a glutamic acid (E), an arginine (R), and or a histidine (H). The core sequence may also comprise non-naturally occurring amino acids (L- and/or D-isomers), e.g. s Beta-alanine (B) and/or an aminohexanoic acid (X). Where the core is a tripeptide motif, it may comprise a glycine (G), a serine (S), and either a cysteine (C) or an alanine (A). Preferably, the core comprises an ionisable residue, such as a histidine (H). The core sequence may comprise an arginine (R), a histidine (H) and a cysteine (C). The core sequence may comprise an arginine (R) or glycine (G), a histidine (H) or serine (S) and a cysteine (C) or an alanine (A). For instance, the core sequence may be GSC or RHC. The tripeptide motif may comprise an alanine (A), a lysine (K) and a leucine (L). For instance, the core sequence may be KLA. The core peptide may be covalently bound to a further moiety, such as a cell specific targeting peptide, or may be derivatized with a lipid molecule. One, some or all of the amino acids of the dendrimer, e.g. of the core peptide motif may be covalently bound to a further moiety, such as an antibody, a cell specific targeting peptide, sugar ligands such as glucose, mannose, galactose and GalNAc (or glycans comprising the same) and/or a lipid substituent. The skilled person is readily able to select further moieties that do not adversely affect solubility or nucleic acid binding characteristics.

The lipid component of the composition may comprise a mixture of lipids, including a cationic lipid. For instance, the lipid component may comprise dioleoylphosphatidylethanolamine (DOPE) and N-[1-(2,3-dioleyloxy)propyl]-N,N,N-trimethylammonium chloride (DOTMA). The DOPE:DOTMA ratio can be readily determined for optimal properties for a given application, but will typically range from 1:5 to 5:1.

Preferably the range is 3:1 to 1:3, or 2:1 to 1:2. Most preferably the DOPE:DOTMA ratio is 1:1. In other embodiments, the lipid comprises 1,2-dioleoyl-3-trimethylammonium-propane chloride (DOTAP), e.g. as the sole lipid or in combination with DOPE.

In other embodiments, the lipid component of the composition may comprise other lipids, in addition to (or instead of) DOPE and DOTMA. Exemplary lipid components are set out below:

Cationic Lipids:

N-[1-(2,3-dioleyloxy)propyl]-N,N,N-trimethylammonium chloride (DOTMA)

1,2-dioleoyl-3-trimethylammonium-propane chloride (DOTAP)

2,3-dioleyloxy-N-(2[spermine-carboxamido]ethyl)-N,N-dimethyl-1-propanaminium trifluoroacetate (DOSPA)

3β-[N—(N′,N′-dimethylaminoethane)-carbamoyl]cholesterol hydrochloride (DC-chol)

Neutral Lipids:

1,2-dioleoyl-sn-glycero-3-phosphoethanolamine (DOPE)

1,2-dioleoyl-sn-glycero-3-phosphocholine (DOPC)

Cholesterol

Anionic Lipids:

1,2-dioleoyl-sn-glycero-3-phospho-(1′-rac-glycerol) (DOPG)

Ionisable Lipids:

DLin-DMA

DLin-KC2-DMA

DLin-MC3-DMA

Other potential lipids include 4-(2-aminoethyl)-morpholino-cholesterol-hemisuccinate, (MoChol) cholesterolhemisuccinate (CHEMS), phosphatidylcholine (PC), phosphatidylethanolamine (PE), dimyristoylphosphatidylcholine (DMPC), dipalmitoylphosphatidylcholine (DPPC), distearoylphosphatidylcholine (DSPC), dimyristoylphosphatidylethanolamine (DMPE), dipalmitoylphosphatidylethanolamine (DPPE), cholesterol-(3-imidazol-1-yl propyl)carbamate (CHIM), dimethyldioctadecylammonium bromide (DDAB), dioleoylphosphatidylserine (DOPS), dioleoylphosphatidylglycerol (DOPG), cholesterol sulfate (chol-SO₄).

It is envisaged that any of the aforementioned lipids can be used alone or in combination with each other in the compositions of the invention. Additionally, the lipids can be derivatized via linkage to PEG group such as PEG2000.

This invention provides methods of delivering nucleic acids into a target cell, the method comprising contacting the target cell with the composition of the invention. The target cell may be a myocyte, a hepatocyte, a stellate cell, a neurons, an astrocyte, a splenocyte, a lung cell, a cardiomyocyte, a kidney cell, an adipose cell, a myeloid derived suppressor cell (MDSC), a tumour associated macrophage or a tumour associated neutrophil, a stem cell or a tumour cell. This method may be performed in vitro. The cell may have been obtained from a patient, and the cell may be administered to the patient after the method has been performed, i.e. the method may be ex vivo. Alternatively, this method may be performed in vivo, as part of a medical use or treatment. The method involves cell entry via clathrin-mediated endocytosis, caveolae-mediated endocytosis and/or micropinocytosis. Such in vivo treatments can involve administering the composition to the subject to deliver of the nucleic acid to a particular target tissue, or tissues; for instance to the muscles and/or liver of the subject. The composition can be administered as a single dose, or as two or more doses.

This invention also provides novel dendrimers such as those shown in Table 2. In some embodiments, the dendrimer is a third-generation dendrimers such as the G1,2,3-LR dendrimer, the G1,2,3-rl dendrimer, the G1-LR,G2,3-RL dendrimer, the G1,2-RL,G3-LR dendrimer, and the G1,2-LR,G3-RL dendrimer, which can be collectively defined as a peptide dendrimer comprising: a first lysine residue and two first dipeptide motifs; two second lysine residues and four second dipeptide motifs; four third lysine residues and eight third dipeptide motifs; and a core peptide sequence which is covalently bound to the first lysine residue, (i) wherein the first lysine residue is covalently bound to the two first dipeptide motifs, which are respectively covalently bound to the two second lysine residues; (II) wherein each second lysine residue is covalently bound to two second dipeptide motifs, wherein each second peptide motif is respectively covalently bound to one of the third lysine residues; and (iii) wherein each third lysine residue is covalently bound to two of the third dipeptide motifs, wherein the first, second and third dipeptide motifs each comprise a leucine (L) and an arginine (R), and wherein at least one of the first, second and third dipeptide motifs consist of a leucine-arginine (LR) dipeptide motif (where the “LR” motif is recited according to the standard N-terminus to C-terminus convention). Each amino acid residue is independently selected from the L-isoform or D-isoform. These novel dendrimers can form part of the compositions of the invention, and can be used in the other aspects of the invention disclosed herein.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Comparison of the transfection efficiency of dendrimers with different lysine and leucine arrangements for DNA delivery. Changing the position of the lysine and leucine within a generation does not alter significantly the transfection efficiency in (A) HeLa or (B) Neuro2A cells. Structural modifications of the dendrimer G1,2,3-KL to its variants such as G1,2,3-K (a version of G1,2,3-KL with all the leucines removed) and substituting all the L-form amino acids to D-form does not vary significantly the transfection efficiency in (C) HeLa or (D) Neuro2A cells. There is no significant difference when G0,1,2,3-KL (a version of G1,2,3-KL with the zero generation bearing the KL motif) in (E) HeLa or (F) Neuro2A cells. Conditions: 1×10⁴ cells were transfected with 0.25 μg pCl-Luc. Error bars refer to the mean±SEM for experiments carried out in triplicate. D/D is DOTMA/DOPE.

FIG. 2. Comparison of the transfection efficiency of dendrimers with different generations (G1, G2 and G3) and cationic residues (e.g. KL vs RL) for DNA delivery. (A) Transfection efficiency in HeLa cells, (B) transfection efficiency in Neuro2A cells. Cells transfected with a luciferase-expressing plasmid (pCl-Luc) in the presence of peptide dendrimers with D/D (measured 24 h after transfection). The luminescence values were normalized by dividing them by the analogous values for cells treated with a D/D DNA complex (w/w 1:1, 0.25 μg) to generate the % of transfection. Conditions: 1×10⁴ cells were transfected with 0.25 μg pCl-Luc. Error bars refer to the mean±SEM for experiments carried out in triplicate. * denotes p<0.05. D/D is DOTMA/DOPE.

FIG. 3. Comparison of the transfection efficiency of G1,2,3-RL with other commercial transfection reagents including DOTMA/DOPE, Polyethylenimine and Lipofectamine 2000 for DNA delivery. (A) Transfection in HeLa cells without the presence of serum, (B) transfection in HeLa cells with the presence of serum, (C) transfection in Neuro2A cells without the presence of serum and (D) transfection in Neuro2A cells with the presence of serum. Cells transfected with a luciferase-expressing plasmid (pCl-Luc) in the presence of G1,2,3-RL with D/D (measured 24 h after transfection). The luminescence values were normalized by dividing them by the analogous values for cells treated with a D/D DNA complex to generate the % of transfection. Conditions: 1×10⁴ cells were transfected with 0.25 μg pCl-Luc for 4 hours without serum or in the presence of 10% serum. Error bars refer to the mean±SEM for experiments carried out in triplicate. G1,2,3-RL mediates transfection significantly more potent than other reagents in HeLa and Neuro2A cells. * denotes p<0.05, *** denotes p<0.001 and **** p<0.0001. D/D is DOTMA/DOPE.

FIG. 4. Comparison of the transfection efficiency of dendrimers with different generations (G1, G2 and G3) and cationic residues (e.g. KL vs RL) for DNA delivery in serum conditions. (A) Transfection efficiency in HeLa cells, (B) transfection efficiency in Neuro2A cells. Cells transfected with a luciferase-expressing plasmid (pCl-Luc) in the presence of peptide dendrimers with D/D (measured 24 h after transfection). The luminescence values were normalized by dividing them by the analogous values for cells treated with a D/D DNA complex (w/w 1:1, 0.25 μg) to generate the % of transfection. Conditions: 1×10⁴ cells were transfected with 0.25 μg pCl-Luc for 4 hours in the presence of 10% serum. Error bars refer to the mean±SEM for experiments carried out in triplicate. G1,2,3-RL (N/Ps between 5-20) mediates transfection significantly more potent than other groups in both HeLa and Neuro2A cells. * denotes p<0.05; G1,2,3-RL (N/Ps at 10, 20) mediates transfection significantly more potent than other groups, *** denotes p<0.001. D/D is DOTMA/DOPE.

FIG. 5. Cellular uptake pathways of the G1,2,3-RL-D/D-DNA nanoparticles. Chlorpromazine inhibits clathrin mediated endocytosis; Genistein and Rottlerin inhibit caveolae-mediated endocytosis and micropinocytosis respectively.

FIG. 6. Follistatin expression in skeletal muscles following intravenous administration of compositions comprising G1,2,3-RL with D/D and plasmid DNA expressing follistatin. Mice were injected with the G1,2,3-RL complexes twice and tissues were harvested to assay the mRNA expression level of follistatin via qPCR.

FIG. 7. Body weight of the mice following intravenous administration of the disclosed compositions.

FIG. 8. Luciferase expression in mice tissue following intravenous administration of compositions comprising G1,2,3-RL or G1,2,3-LR with DOTMA/DOPE (w/w=1:1 to DNA) and mini-circle DNA expressing luciferase. The compositions was injected at an NP ratio of 20:1. Mice were injected with the compositions and 24 hours later the tissues were harvested to measure luciferase signal in a heart, kidney, liver, lung, spleen and skeletal muscle.

FIG. 9. Luciferase expression in mice skeletal muscle following intramuscular administration of composition comprising G1,2-RL, 3-LR with DOTMA/DOPE (w/w=1:1 to DNA) and mini-circle DNA expressing luciferase. The composition was injected at an NP ratio of 20:1. Mice were injected with the compositions and 48 hours later the skeletal muscle tissues were harvested to measure luciferase signal.

FIG. 10. Luciferase expression in mice tissue following intravenous administration of compositions comprising G1,2,3-RL (top panel) or G1,2-RL, 3-LR (bottom panel) with DOTMA/DOPE (w/w=10:1 to mRNA) and mRNA expressing luciferase. Each dendrimer composition was injected at an NP ratio of 8:1 and 0.15:1. Mice were injected with the compositions and 6 hours later the tissues were harvested to measure luciferase signal in muscle (gastrocnemius), liver, lung, heart, spleen, kidney, adipose tissues, brain, cervical lymph nodes and inguinal lymph nodes. Compositions comprising G1,2,3-RL and G1,2-RL, 3-LR are capable of delivering mRNA to immune cell rich tissues including Lung, Spleen, and Lymph Nodes when injected at an NP ratio of 0.15:1. G1,2-RL, 3-LR injected at NP 8:1 is able to target delivery of mRNA to spleen.

FIG. 11. Luciferase expression in mice tissue following intravenous administration of compositions comprising G1,2,3-RL (top panel) or G1,2-RL, 3-LR (bottom panel) with DOTMA/DOPE (w/w=10:1 to mRNA) and mRNA expressing luciferase. Each dendrimer composition was injected at an NP ratio of 8:1 and 0.15:1. Mice were injected with the compositions and 6 hours later the tissues were harvested to measure luciferase signal in muscle (gastrocnemius), liver, heart, kidney, adipose tissues and brain.

FIG. 12. Comparison of luciferase expression in mice tissue following intravenous administration of compositions comprising G1,2,3-RL or G1,2-RL, 3-LR with DOTMA/DOPE (w/w=10:1 to mRNA) and mRNA expressing luciferase at an NP ratio of 8:1. Mice were injected with the compositions and 6 hours later the tissues were harvested to measure luciferase signal in muscle (gastrocnemius), liver, lung, heart, spleen, kidney, adipose tissues, brain, cervical lymph nodes and inguinal lymph nodes (FIG. 12A, top panel). The same data is presented to allow for comparison between lung, cervical lymph nodes and inguinal lymph nodes (FIG. 12A, bottom panel) and muscle (gastrocnemius), liver, heart, kidney, adipose tissues and brain (FIG. 12B). Compositions comprising G1,2-RL, 3-LR injected at an NP ratio 8:1 are capable of delivering mRNA to immune cell-rich tissues with higher efficiency compared to G1,2,3-RL injected at an NP ratio 8:1.

FIG. 13. Luciferase expression in mice tissue following intravenous administration of two doses of a composition comprising G1,2,3-RL with DOTMA/DOPE (w/w=10:1 to mRNA) and mRNA expressing luciferase. The dendrimer composition was injected at an NP ratio-0.15:1. Mice were injected with the compositions and 6 hours later the tissues were harvested to measure luciferase signal in muscle (gastrocnemius), liver, lung, heart, spleen, kidney, adipose tissues, brain, cervical lymph nodes and inguinal lymph nodes.

FIG. 14. Luciferase expression in mice tissue following intravenous administration of compositions comprising G1,2,3-RL, G1,2-RL, 3-LR or NTX1 with DOTMA/DOPE (w/w=10:1 to mRNA) and mRNA expressing luciferase. Each dendrimer composition was injected at an NP ratio of 0.15:1. Mice were injected with the compositions and 6 hours later the tissues were harvested to measure luciferase signal in muscle (gastrocnemius), liver, lung, heart, spleen, kidney, adipose tissues, brain, cervical lymph nodes and inguinal lymph nodes. Compositions comprising NTX1, which comprises a skeletal muscle targeting domain and a cell penetrating peptide domain, is capable of delivering mRNA to immune cell-rich tissues with higher efficiency compared to compositions comprising G1,2,3-RL and G1,2-RL, 3-LR.

FIG. 15. Luciferase expression in mice tissue following intravenous administration of compositions comprising G1,2,3-RL, G1,2-RL, 3-LR or NTX1 with DOTMA/DOPE (w/w=10:1 to mRNA) and mRNA expressing luciferase and compared to mice injected with a composition comprising DOTMA/DOPE (w/w=10:1 to mRNA) and mRNA expressing luciferase without dendrimer. Each dendrimer composition was injected at an NP ratio of 0.15:1. Mice were injected with the compositions and 6 hours later the tissues were harvested to measure luciferase signal in muscle (gastrocnemius), liver, lung, heart, spleen, kidney, adipose tissues, brain, cervical lymph nodes and inguinal lymph nodes.

FIG. 16. Luciferase expression in mice tissue following intravenous administration of compositions comprising G1,2-RL, 3-LR with DOTMA/DOPE (w/w=10:1 to mRNA) and mRNA expressing luciferase and compared to mice injected with a composition comprising DOTMA/DOPE (w/w=10:1 to mRNA) and mRNA expressing luciferase without dendrimer. The dendrimer composition was injected at an NP ratio of 8:1. Mice were injected with the compositions and 6 hours later the tissues were harvested to measure luciferase signal in muscle (gastrocnemius), liver, lung, heart, spleen, kidney, adipose tissues, brain, cervical lymph nodes and inguinal lymph nodes. Compositions comprising NTX1, G1,2,3-RL and G1,2-RL, 3LR each show increased delivery of mRNA to all tissues relative to compositions comprising mRNA alone or compositions comprising DOTMA/DOPE and mRNA.

FIG. 17. Luciferase and eGFP expression in HeLa or C2C12 cells following transfection with dendrimer compositions or commercially available transfection reagents. HeLa cells were transfected with 1) luciferase mRNA alone, a composition comprising G1,2-RL, 3-LR (NP=0.16:1), DOTMA/DOPE (w/w=10:1 to mRNA) and luciferase mRNA or a composition comprising Lipofectamine 2000™ and Luciferase mRNA (top left panel): or 2) eGFP mRNA alone, a composition comprising G1,2-RL, 3-LR (NP=0.18:1), DOTMA/DOPE (w/w=10:1 to mRNA) and eGFP mRNA or a composition comprising DLin-MC3-DMA: Cholesterol: DSPC: DMG-PEG lipid nano particle and eGFP mRNA (top right panel). C2C12 cells were transfected with luciferase mRNA alone, a composition comprising G1,2-RL, 3-LR (NP=8:1), DOTMA/DOPE (w/w=10:1 to mRNA) and luciferase mRNA, a composition comprising Lipofectamine 2000™ and luciferase mRNA or polyethylenimine and luciferase mRNA (bottom panel). Luciferase and eGFP expression was measured 24 hours after transfection. Peptide dendrimers of the present invention are more efficient at transfecting HeLa cells in vitro compared to commercially available transfection reagents, such as Lipofectamine 2000 and LNPs.

FIG. 18. RNA transfection efficiency in HeLa cells following transfection with fluorescently labelled sgRNA and Cas9 mRNA or G1,2-RL, 3-LR with fluorescently labelled sgRNA and Cas9 mRNA at an NP ratio of 0.15:1 or 8:1. The sgRNA was labelled with TM-Rhodamine and Cas9 mRNA was labelled with Cy5 fluorophore. Cells were transfected for 2 hours and assayed by flow cytometry. 100% of HeLa cells exposed to compositions comprising G1,2-RL, 3LR are double positive for sgRNA and Cas9 mRNA demonstrating an extremely high transfection efficiency.

FIG. 19. RNA transfection efficiency in HeLa cells following transfection with tracrRNA (trRNA), crRNA and Cas9 mRNA or G1,2-RL, 3-LR with tracrRNA, crRNA and Cas9 mRNA at an NP ratio of 0.15:1 or 8:1. The tracrRNA was labelled with ATTO 55 fluorophore. crRNA was labelled with fluorescein, and Cas9 mRNA was labelled with Cy5 fluorophore. Cells were transfected for 2 hours and assayed by flow cytometry.

FIG. 20. RNA transfection efficiency in HeLa cells following transfection with either tracrRNA (trRNA), crRNA or Cas9 mRNA. Cells were transfected with or without G1,2-RL, 3-LR. The tracrRNA was labelled with ATTO 55 fluorophore. crRNA was labelled with fluorescein, and Cas9 mRNA was labelled with Cy5 fluorophore. Cells were transfected for 2 hours and assayed by flow cytometry.

FIG. 21. mRNA delivery to immune cells in the spleen. Mice were injected intravenously with mRNA alone, or mRNA with either NTX1 (NP=0.15:1), G1,2-RL, 3-LR (NP=0.15:1) or G1,2-RL, 3-LR (NP=8:1). mRNA was labelled with AlexaFluor488. Spleens were harvested from mice 2 hours post-injection for processing. Delivery of mRNA to a wide range of immune cells present in the spleen is possible using compositions comprising NTX1, G1,2-RL, 3-LR (NP 8:1) or G1,2-RL, 3-LR (NP 0.15:1).

FIG. 22. mRNA delivery to immune cells in the bone marrow. Mice were injected intravenously with mRNA alone, or mRNA with either NTX1 (NP=0.15:1), G1,2-RL, 3-LR (NP=0.15:1) or G1,2-RL, 3-LR (NP=8:1). mRNA was labelled with AlexaFluor488. Bone marrow was harvested from mice 2 hours post-injection for processing.

FIG. 23. mRNA delivery to immune cells in the blood. Mice were injected intravenously with mRNA alone, or mRNA with either NTX1 (NP=0.15:1), G1,2-RL, 3-LR (NP=0.15:1) or G1,2-RL, 3-LR (NP=8:1). mRNA was labelled with AlexaFluor488. Blood was harvested from mice 2 hours post-injection for processing. Delivery of mRNA to a wide range of immune cells present in the blood is possible using compositions comprising NTX1, G1,2-RL, 3-LR (NP 8:1) or G1,2-RL, 3-LR (NP 0.15:1).

FIG. 24. mRNA delivery to immune cells in the lymph nodes. Mice were injected intravenously with mRNA alone, or mRNA with either NTX1 (NP=0.15:1), G1,2-RL, 3-LR (NP=0.15:1) or G1,2-RL, 3-LR (NP=8:1). mRNA was labelled with AlexaFluor488. Lymph nodes were harvested from mice 2 hours post-injection for processing.

FIG. 25. In vitro DNA delivery to HeLa and C2C12 cells. HeLa cells were transfected with either DNA alone, G1,2,3-RL (N:P=5:1) and DOTMA/DOPE (at a w/w=1:1 to DNA) with DNA and G1,2,3-RL (N:P=5:1) and DOPG/DOPE (at a w/w=1:1 to DNA) with DNA (top panel). C2C12 cells (a muscle-derived cell line) were transfected with either DNA alone, polyethylenimine with DNA, G1,2-RL, 3-LR (N:P=8:1) and DOTMA/DOPE (at a w/w=1:1 to DNA) with DNA and NTX2 (N:P=8:1) and DOTMA/DOPE (at a w/w=1:1 to DNA) with DNA (bottom panel). The DNA used in both experiments encodes a luciferase reporter gene. NTX2 is a G1,2-RL, 3-LR dendrimer conjugated to a muscle targeting peptide ASSLINA ((LR)8(KRL)4(KRL)2KGSCGAASSLNIA(Acp)-NH2).

FIG. 26. Aspartate transaminase (AST; 26A, top panel), TNF-α (26A, bottom panel), IL-6 (26B, top panel) and IL-1β (26B, bottom panel) levels following mRNA formulations dosing. Mice were treated with either mRNA alone or with mRNA, dendrimer and DOTMA/DOPE (w/w=10:1 to mRNA). The N:P ratios of the dendrimer to mRNA are indicated in the X-axis. All the formulations were dosed once apart from G1,2,3-RL and G1,2-RL, 3-LR which were also dosed twice. For mice injected with 2 doses, the 1st dose was injected 24 hours prior to the 2nd dose. The plasma of the mice was harvested 6 hours after the 2nd dose for the measurement of the AST or cytokine levels. For mice dosed for 1 time, the plasma was harvested 6 hours after dosing for the measurement of the AST and cytokine levels. Using the dendrimers of the invention to deliver mRNA in vivo does not cause significant liver toxicity or a significant immune reaction.

FIG. 27. Aspartate transaminase (top left panel), TNF-α (top right panel), IL-6 (bottom left panel) and IL-1β (bottom right panel) levels following DNA formulation dosing. Mice were treated with either DNA alone or with G1,2,3-RL and DOTMA/DOPE (w/w=1:1 to DNA). All the formulations were dosed twice before plasma samples were harvested and AST or cytokine levels measured. Using the dendrimers of the invention to deliver DNA in vivo does not cause significant liver toxicity or a significant immune reaction.

FIG. 28. mRNA transfection of the peptide dendrimers with lipid system in vitro. HeLa cells were transfected with mRNA alone or mRNA with peptide dendrimers (N:P=0.16:1) and DOTMA/DOPE (w/w=10:1 to mRNA). The mRNA used expressed an eGFP reporter gene. Following a 24 hour transfection, the cells were harvested to assay the eGFP expression. The eGFP expression was normalised with the total amount of protein of each transfection. The gene expression level was calculated by normalising the transfection value of the G1,2-RL, 3-LR, DOTMA/DOPE & mRNA treated cells. 1^{st}, 2^{nd} and 3^{rd} generation dendrimers with varying cores and 1^{st}, 2^{nd}, and 3^{rd} generation peptide motifs can transfect cells with mRNA effectively under the tested conditions.

FIG. 29. mRNA transfection of the peptide dendrimers with lipid system in vitro. HeLa cells were transfected with mRNA alone or mRNA with peptide dendrimers and DOTMA/DOPE (w/w=10:1 to mRNA). The N:P ratio of G1,2-RL,3-LR was at 0.16:1 while the N:P ratios of all other dendrimers at 8:1. The mRNA used expressed an eGFP reporter gene. Following a 24 hour transfection, the cells were harvested to assay the eGFP expression. The eGFP expression was normalised with the total amount of protein of each transfection. The gene expression level was calculated by normalising the transfection value of the G1,2-RL, 3-LR, DOTMA/DOPE & mRNA treated cells. 1^{st}, 2^{nd} and 3^{rd} generation dendrimers with varying cores and 1^{st}, 2^{nd}, and 3^{rd} generation peptide motifs can transfect cells with mRNA effectively under the tested conditions.

FIG. 30. Luciferase expression in mice tissue following intravenous administration of compositions comprising G1,2-RL, 3-LR or RHCG1-RL, 2-LR with DOTMA/DOPE (w/w=10:1 to mRNA) and mRNA expressing luciferase. The dendrimer composition was injected at either an N:P ratio of 0.15:1 or 8:1. Mice were injected with the compositions and 6 hours later the tissues were harvested to measure luciferase signal in muscle (gastrocnemius), liver, lung, heart, spleen, kidney, adipose tissues, brain, and lymph nodes. Generation 2 dendrimers are capable of targeting mRNA delivery to the spleen and lung with higher efficiency than the third generation dendrimer G1,2-RL, 3-LR.

FIG. 31. Third generation dendrimers are represented diagrammatically (with N-termini on the left and C-terminus on the right).

### DETAILED DESCRIPTION OF THE INVENTION

Aspects and embodiments of the present invention will now be discussed with reference to the above-identified figures and the technical definitions that follow below. Further aspects and embodiments will be apparent to those skilled in the art. All documents mentioned in this text are incorporated herein by reference.

Current immunotherapy response rates are around 15-20%, there is an urgent need to improve the treatment outcome. One strategy is to deliver mRNA to express proteins (such as CEBPA, IRF8, cGAS-STING, SOCS1 and/or SOCS3) to revert the immunosuppressive phenotypes of myeloid cells in the tumour microenvironment, which would provide a more favourable environment for immunotherapy to be responsive. Another strategy can be to transfer mRNA into the immune cells in the tumour to express cytokines (such as IL-2, IL-7, IL-12, IL-15, IL-21 and/or interferon) to activate the immune cells to fight against cancer cells. It is also possible to deliver mRNA to the macrophage to express a chimeric antigen receptor so that the macrophage can be activated to kill tumour cells. Delivering mRNA to express tumour antigens in antigen presenting cells would help activate the immune system to attack cancer cells. These strategies can be applied to treat all tumours, especially Non-small cell lung cancers, Advanced melanoma, Prostate cancer, Ovarian cancer, Breast cancer, Lung cancer, Bile duct cancer (Cholangiocarcinoma), Gallbladder cancer, Neuroendocrine tumors, Hepatocellular carcinoma, Colorectal cancer, Pancreatic cancer and Solid tumors.

Delivery of Nucleic Acids

The compositions of the invention can be used to deliver the nucleic acids described herein to certain tissues of the human or animal body. For instance deliver to the following tissues:
1, Skeletal muscle
2, Liver
3, Lung
4, Heart
5, White adipose tissues
6, Brown adipose tissues
7, Brain
8, Spleen
9, Bone marrow
10, Joints
11, Kidney
12, GI tract
13, Tumour
14, Eyes
15, Thymus
16, Skin
17, Lymph nodes
18, Pancreas
19, Adrenal gland
20, Testis
21, Prostate
22, Ovary
23, Uterus
24, Bladder
25, Diaphragm

mRNA Transfection

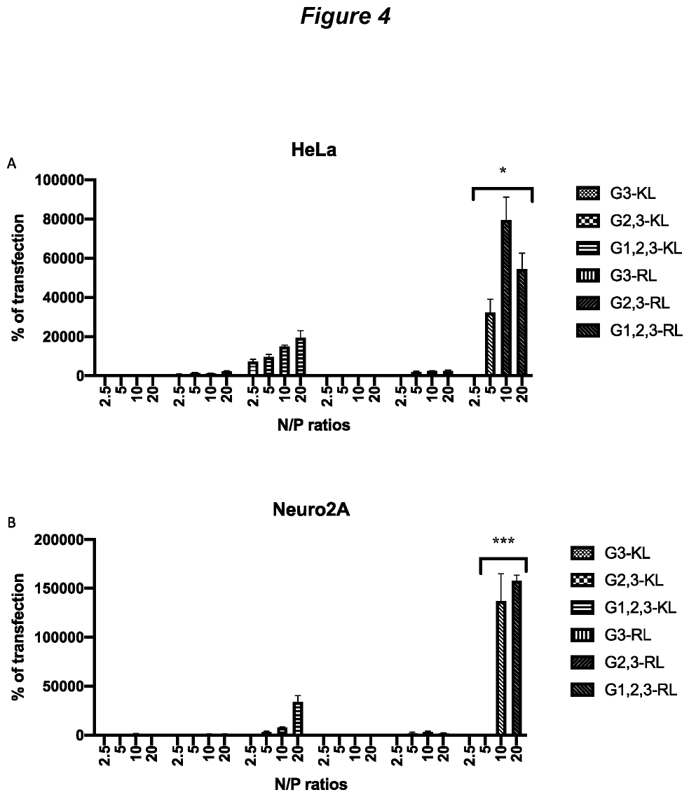

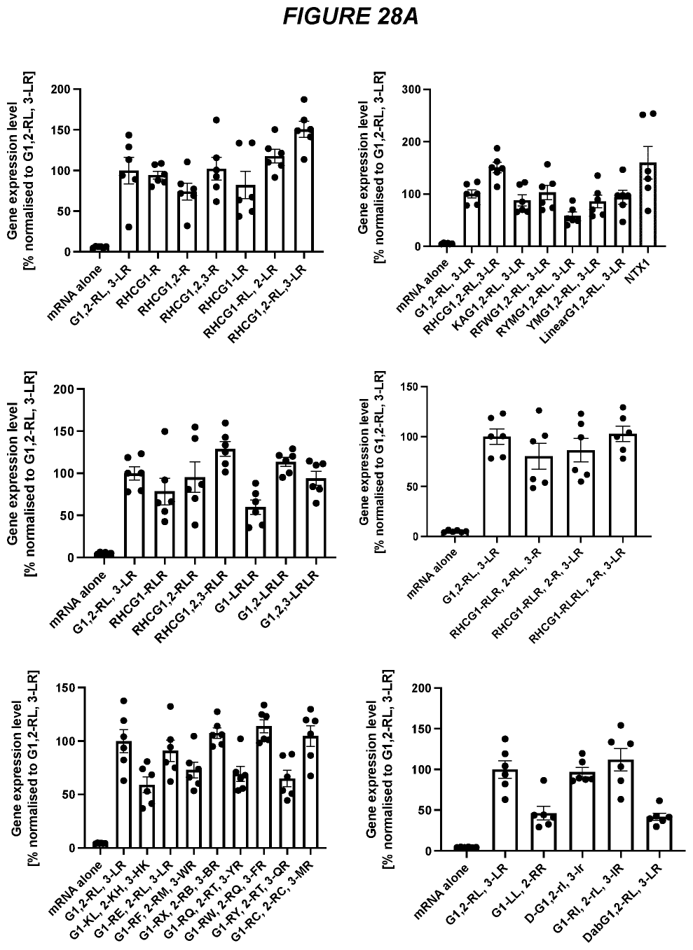

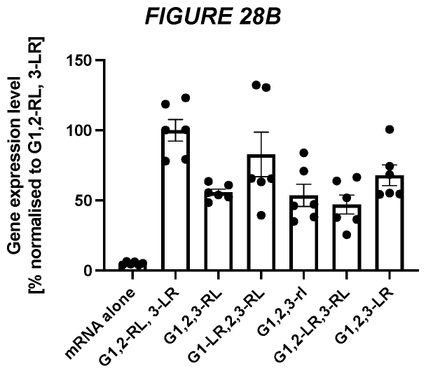

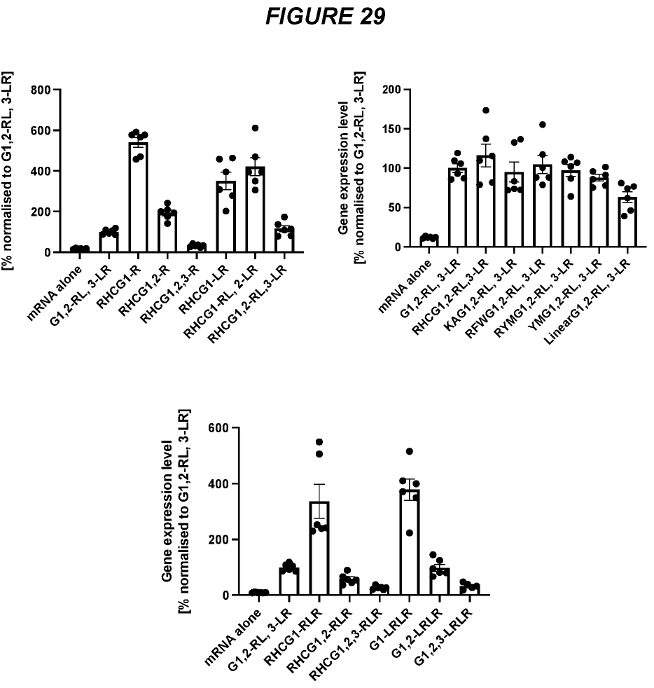

The effect on the number of generations of dendrimers on mRNA delivery was studied by transfecting HeLa cells in full growth medium conditions. We have shown that dendrimers with 1 or 2 generations (e.g. RHCG1-R, RHCG1,2-R, RHCG1-LR, RHCG1-RL, 2-LR) can transfect cells effectively and mediate similar transfection efficiency as dendrimers with 3 generations (FIG. 28A). This is indeed very different to DNA transfection because dendrimers with 1 or 2 generations do not transfect cells as well as dendrimers with 3 generations in full growth medium (FIG. 4). Interestingly, increasing the N:P ratios of the generation 1 or generation 2 dendrimers from 0.16 to 8 further increases the transfection efficiency (FIG. 29A).

The inventors studied the effect of the core sequences of the dendrimers on transfection and have shown tripeptide sequences such as GSC (e.g., from G1,2-RL,3-LR) to be effective. Dipeptide core sequences, such as KA and YM are also effective. This change in core peptide length does not affect mRNA transfection, suggesting that dendrimers with 2 amino acids in the core would transfect as well as dendrimers with 3 amino acids in the core (FIG. 28A). Different tripeptide core sequences such as RFW, RYM, perform comparably to GSC. Although an arginine, which contains a cationic group, is added to the core, it does not improve or decrease transfection. Interestingly, when the 3 amino acids are changed to RHC from GCS at the core, transfection can improve by 50%. This suggests that an ionisable group such as histidine in the core of the dendrimers can improve transfection (FIG. 28A).

The use of 12 amino acid core sequences (e.g., dendrimer ‘LinearG1,2-RL, 3-LR’) demonstrate that transfection efficiency is not impacted by increasing the length of the core sequence (FIG. 28A). This result indicated that it is possible to introduce a longer amino acid chain and/or a longer structure in the core without affecting transfection efficiency. Indeed, NTX1, which contains 27 amino acids in the core, transfects more effectively than G1,2-RL, 3-RL. This demonstrates that a longer core sequence can indeed enhance transfection. It is likely that the transfection enhancement from NTX1 is due to the presence of the cell penetrating peptide within the core sequence.

The inventors explored the effect on the number of amino acids within the generations on transfection. Thus we tested dendrimers with only 1 amino acid (R), 2 amino acids (RL or LR), 3 amino acids (RLR) and 4 amino acids (LRLR). These studies indicated that dendrimers with 1, 2, 3 or 4 amino acids in each generation can still transfect mRNA well into cells (FIG. 28A). Even using dendrimers with different numbers of amino acids in each generation showed that transfection efficiency is not affected.

Based on the G1,2-RL, 3-LR structure, the inventors designed a library of 3 generation dendrimers in which we have replaced the basic amino acid R to K, and/or changing the hydrophobic amino acid L to an acidic amino acid such as E, and/or an amino acid with a non-polar side chain such as M, F, beta-alanine (B), aminohexanoic acid (X) and W and/or an amino acid with a polar side chain such as Q, T and Y.

Replacing R to K within the dendrimers reduces the mRNA transfection efficiency (FIG. 28A). However, changing the hydrophobic amino acids to other hydrophobic amino acids and/or amino acids with polar or non-polar side chains may not affect transfection or may reduce transfection by 30-40%, as compared to G1,2-RL, 3-LR. However, all these dendrimers can induce mRNA transfection significantly higher than the mRNA alone control.

The inventors also investigated the impact of L- or D-form amino acids within the dendrimers on mRNA transfection. Based on the G1,2-RL, 3-LR dendrimer, we find that changing part or all the amino acids from L to D-form in each generation of the dendrimers would not affect transfection efficiency. Substituting the lysine to the diaminobutyric acid within the dendrimer would reduce transfection, although the transfection of this dendrimer is still significantly higher than the mRNA alone control.

The inventors demonstrated that G1-LL, 2-RR can be used to deliver mRNA with our formulation protocol, in which we used G1-LL, 2-RR at a 0.16:1 N:P ratio with DOTMA/DOPE (w/w 10:1). Interestingly, this dendrimer was used to deliver ASO in vitro and in vivo in a different formulation (Saher 2018). The formulation used was DOTMA/DOPE (w/w=2:1 to ASO) and N:P=20:1, G1-LL, 2-RR to ASO. We have tried this on transfecting cells (i.e. DOTMA/DOPE (w/w=2:1 to mRNA) and N:P=20:1, G1-LL, 2-RR to mRNA) and the transfection efficiency is poor. This formulation (DOTMA/DOPE (w/w=2:1 to mRNA) and N:P=20:1, G1-LL, 2-RR to mRNA) only yields 10% of the mRNA transfection efficiency of our improved formulation (DOTMA/DOPE (w/w=10:1 to mRNA) and N:P=0.16:1, G1-LL, 2-RR to mRNA).

The inventors have explored the 3 generation dendrimers with either RL or LR or rl in different generations for mRNA delivery. We found that most of these dendrimers transfect cells similarly, with G1,2-RL, 3-LR being the most effective in mRNA transfection. Overall, our data suggested that dendrimer with hydrophobic and cationic amino acid in each generation would yield effective mRNA delivery to the cells.

Since there are 1^{st} and 2^{nd} generation dendrimers transfecting cells more effective than G1,2-RL, 3-LR, we have selected these dendrimers for further testing on transfection. These dendrimers can in general transfect better than G1,2-RL, 3-LR in various N:P ratios. In particular, RHCG1-R, RHCG1,2-R, RHCG1-LR, RHCG1-RL, 2-LR, RHCG1-RLR and G1-LRLR at an N:P=4:1 can transfect 700% to 1815% better than G1,2-RL, 3-LR. These data demonstrated that the requirement for DNA and mRNA delivery is very different. For mRNA, there is a trend in which dendrimer with 1 or 2 generation would transfect cells better than 3 generation dendrimers in full growth medium conditions. However, generation 3 dendrimer would transfection cells with DNA way better than generation 1 or 2 dendrimers in full growth medium conditions (FIG. 4).

Certain dendrimers of the invention that are particularly preferred for mRNA delivery include RHCG1-R, RHCG1,2-R, RHCG1-LR, RHCG1-RL,2-LR, G1,2-RL,3-LR, RHCG1-RLR, RHCG1,2-RLR, G1-LRL, RHCG1,2-RL,3-LR, and G1,2,3-RL.

RNA Interference

The present invention facilitates the therapeutic down regulation of target gene expression via delivery of nucleic acids. These include RNA interference (RNAi). Small RNA molecules may be employed to regulate gene expression.

These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA Interference (RNAI), is a phenomenon in which dsRNA complexes can target specific genes of homology for silencing in a short period of time. It acts as a signal to promote degradation of mRNA with sequence identity. A 21-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, the present invention provides the use of these sequences in a composition of the invention for down-regulating the expression of a target gene.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed on John et al, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such as Ambion's online siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers et al (2003)). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thloate); P(S)S, (dithioate); P(O)NR′2; P(O)R′; P(O)OR6; CO; or CONR′2 wherein R is H (or a salt) or alkyl (1-12 C) and R6 is alkyl (1-9 C) Is joined to adjacent nucleotides through-O-or-S—.

Long Non-Coding RNA

Mammalian genomes are pervasively transcribed, producing a vast array of transcripts including many thousands of long non-coding RNA molecules (lncRNAs). It has been shown that lncRNAs can regulate the chromatin state, transcription, RNA stability, and the translation of certain genes.

RNA Activation (RNAa)

RNA activation (RNAa) is a process mediated by RNAs to enhance gene expression via a highly regulated and evolutionarily conserved pathway. RNAa can be induced by small activating RNA (saRNA), which is a class of noncoding RNA consisting of a 21-nucleotide dsRNA with 2-nucleotide overhangs at both ends. saRNA has an identical structure and chemical components to siRNA despite the fact that saRNA mediates gene activation in a sequence specific manner. To activate gene expression, the guide strand of the saRNA is loaded to AGO2, and the complex is then transported to the nucleus. Once in the nucleus, the guide strand-AGO2 complex binds directly to gene promoters or associated transcripts, recruiting key components including RNA polymerase II to initiate gene activation (Kwok et al. 2019).

Antisense Oligonucleotides (ASOs)

Antisense oligonucleotides (ASOs) are single strands of DNA or RNA that are complementary to a target sequence. The ASO hybridises with the target nucleic acid. For instance, an ASO can be used to target a coding or non-coding RNA molecule in the cell. Following target binding, the ASO/target complex may be enzymatically degraded, e.g. by RNase H.

Messenger RNA (mRNA)

The skilled person is aware that messenger RNA (mRNA) is a single-stranded molecule of RNA that takes the coding sequence of a gene to be translated into the corresponding amino acid sequence by a ribosome. mRNA is created during the process of transcription, where an enzyme (RNA polymerase) converts the gene into primary transcript mRNA (also known as pre-mRNA). This pre-mRNA usually still contains introns, regions that will not go on to code for the final amino acid sequence. These are removed in the process of RNA splicing, leaving only exons, regions that will encode the protein. This exon sequence constitutes mature mRNA. Mature mRNA is then read by the ribosome, thereby producing the encoded protein. The invention can be used to deliver mRNA molecules to target cells and tissues as a means of inducing expression of a desired protein or peptide. Inducing peptide/protein expression via mRNA delivery is particularly useful when transient expression is desired.

mRNAs and lncRNAs are typically large molecules with a negatively charged side and a hydrophobic side. mRNAs and lncRNAs will therefore require a balance between hydrophobic and hydrophilic interactions to be encapsulated and delivered to target tissues and cells. This balance between hydrophobic and hydrophilic interactions will be different from, for example, double stranded nucleic acid such as pDNA and siRNA which has charge on both sides. As mRNAs and lncRNAs are significantly larger than, for example, ASOs the requirement for encapsulation and delivery will also likely be different. As such the optimal NP ratio of dendrimer and w/w ratio of DOTMA/DOPE for mRNA and lncRNA delivery will differ compared to ASO delivery.

### Modified Nucleic Acids

Modified nucleotide bases can be used in addition to the naturally occurring bases, and may confer advantageous properties on nucleic acids containing them.

For example, modified bases may increase the stability of the nucleic acid molecule, thereby reducing the amount required. The provision of modified bases may also provide nucleic acid molecules which are more, or less, stable than unmodified nucleic acids.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 7 substituted sugars such as 7-O-methyl-; 2′-O-alkyl; 2′-O-allyl; 2′-S-alkyl; 2′-S-allyl; 2′-fluoro-; 2′-halo or azido-ribose, carbocyclic sugar analogues, a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-NB-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine, 1-methylcytosine.

Nucleic Acid-Based Vaccines

DNA vaccines, as defined by the World Health Organisation (WHO), and RNA vaccines involve the direct introduction Into appropriate tissues (of the subject to be vaccinated) a plasmid containing the DNA sequence or RNA encoding the antigen(s) against which an immune response is sought, and relies on the in situ production of the target antigen. These approaches offer a number of potential advantages over traditional approaches, including the stimulation of both B- and T-cell responses, improved vaccine stability, the absence of any infectious agent and the relative ease of large-scale manufacture. As proof of the principle of DNA vaccination, immune responses in animals have been obtained using genes from a variety of infectious agents, including influenza virus, hepatitis B virus, human immunodeficiency virus, rabies virus, lymphocytic chorio-meningitis virus, malarial parasites and mycoplasmas. In some cases, protection from disease in animals has also been obtained. However, the value and advantages of DNA vaccines must be assessed on a case-by-case basis and their applicability will depend on the nature of the agent being immunized against, the nature of the antigen and the type of immune response required for protection.

The field of DNA and RNA vaccination is developing rapidly. Vaccines currently being developed use not only DNA, but also include adjuncts that assist DNA to enter cells, target it towards specific cells, or that may act as adjuvants in stimulating or directing the immune response. As of 2020, the WHO noted that the first nucleic acid vaccines licensed for marketing were likely to use plasmid DNA derived from bacterial cells, but that, in future, others may use RNA or may use complexes of nucleic acid molecules and other entities. However, with the onset of the COVID-19 pandemic in 2020, a concerted effort was made to bring the first RNA-based, COVID-19 vaccines to market and these were approved for use in mid- to late-2020. Since approval, these RNA-based vaccines have been successfully rolled out worldwide to immunize the population against COVID-19.

Intramuscular delivery of DNA vaccines, in common with other vaccine technologies, is a common approach (Lim et at, 2020). The low replication rate of myocytes (muscle cells) in the skeletal muscle makes this an attractive target for DNA vaccination, because stable expression does not rely on genomic integration.

The RNA vaccines on the market currently use mRNA encoding the antigen as a payload. An area now being explored to increase the effectiveness of RNA vaccines is the use of self-amplifying RNA. self-amplifying RNA shares many of the structural features of mRNA and may include a 5′ cap, 3′ polyA tall and 5 and 3′ untranslated regions (UTRs). In addition to encoding the antigen of interest a self-amplifying RNA will also comprise a system for self-amplification. For example, a self-amplifying RNA may also encode an RNA-dependent RNA polymerase (RDRA), a promoter and the antigen of interest. Upon translation of an RDRA by the subjects translation machinery, the RDRA can engage the self-amplifying RNA and replicate the RNA. Including a system for self-amplification reduces the minimal RNA required in a vaccine and as a result will reduce the likelihood of a subject experiencing side effects.

Medical Therapies: Gene Therapy

The present invention contemplates use in gene therapy regimens. The nucleic acid can be present in a composition which, when introduced into target cells, results in expression of a therapeutic gene product, e.g. a transgene. Target cells include myocytes, hepatocytes, stellate cells, brain cells (neurons, astrocytes), splenocytes, lung cells, cardiomyocytes, kidney cells, adipose cells, stem cells, monocytes, macrophages, dendritic cells, neutrophils, B cell, T cell, myeloid derived suppressor cells, tumour associated macrophages, tumour associated neutrophils or tumour cells.

For gene therapy to be practical, it is desirable to employ a DNA/RNA transfer system that: (1) directs the therapeutic sequence into the target cell, (2) mediates uptake of the therapeutic nucleic acid into a proportion of the target cell population, and (3) is suited for use in vivo for therapeutic application.

The nucleic acid-containing compositions of the invention can be stored and administered in a sterile pharmaceutically acceptable carrier. Various sterile solutions may be used for administration of the composition, including water, PBS, ethanol, lipids, etc. The concentration of the DNA/RNA will be sufficient to provide a therapeutic dose, which will depend on the efficiency of transport into the cells.

Actual delivery of the gene sequence, formulated as described above, can be carried out by a variety of techniques including direct injection, instillation of lung and other epithelial surfaces, or by intravenous injection. Administration may be by syringe needle, trocar, cannula, catheter, etc, as a bolus, a plurality of doses or extended infusion, etc.

Gene Editing

The present invention contemplates use in gene editing therapies, including gene editing therapies using technologies those well known in the art such as CRISPR/Cas (e.g. CRISPR/Cas9 systems), TALENS and Zinc finger nucleases.

In some embodiments, the CRISPR/Cas system comprises a Cas nuclease, a crispr RNA (crRNA) and a trans-activating crRNA (trRNA or tracrRNA). In this system, the crRNA comprises a sequence complementary to the target DNA and serves to direct the Cas nuclease to the target site in the genome and the tracrRNA serves as a binding scaffold for the Cas nuclease which is required for Cas activity. In some embodiments, the CRISPR/Cas system comprises a Cas nuclease and a single-guide RNA (sgRNA) to direct the Cas nuclease to the target site in the target gene. An sgRNA comprises a target-specific crRNA fused to a scaffold tracrRNA in a single nucleic acid.

In some embodiments, the nucleic acid comprises a DNA or an mRNA encoding a Cas protein or peptide, for example a Cas9 protein or peptide. In some embodiments, the nucleic acid comprises an sgRNA. In some embodiments, the nucleic acid comprises a crRNA and/or a tracrRNA. In some embodiments, the nucleic acid comprises a DNA or mRNA encoding a Cas protein or peptide, a crRNA and a tracrRNA. In some embodiments, the nucleic acid comprises a DNA or mRNA encoding a Cas protein or peptide and a sgRNA.

The CRISPR/Cas system can also be used to direct repair or modification of a target gene. For example, the CRISPR/Cas system can include a nucleic acid template to promote DNA repair or to introduce an exogenous nucleic acid sequence into the target gene by, for example, promoting homology directed repair. The CRISPR/Cas system may also be used to introduce a targeted modification to the target genomic DNA, for example using base editing technology. This can be achieved using Cas proteins fused to a base editor, such as a cytidine deaminase, as disclosed in, for example, WO2017070633A2 which is incorporated by reference. In another example, the CRISPR/Cas system may be used to “rewrite” a nucleic acid sequence in a genome. For example, the CRISPR/Cas system may be a Prime editing system. In such a prime editing system, a fusion protein may be used. For example, the fusion protein may comprise a catalytically impaired Cas domain (e.g. a “nickase”) and a reverse transcriptase. The catalytically impaired Cas domain may be capable of cutting a single strand of DNA to produce a nicked DNA duplex. A Prime editing system may include a prime editing guide RNA (pegRNA) which includes an extended sgRNA comprising a primer binding site and a reverse transcriptase template sequence. Upon nicking of the DNA duplex by the catalytically impaired Cas, the primer binding site allows the 3′ end of the nicked DNA strand to hybridize to the pegRNA, while the RT template serves as a template for the synthesis of edited genetic information.

In some embodiments, the CRISPR/Cas gene editing system may include a nucleic acid template to direct repair of the target gene of interest. In other embodiments, the Cas protein or peptide may include a base editor. In still further embodiments, the CRISPR/Cas system may be a prime editing system.

CRISPR/Cas Gene Silencing and Gene Activation

CRISPR/Cas systems have been adapted for use in gene silencing and activation. Such systems are envisaged for use with the current invention. For example, in some embodiments, the nucleic acid may encode a fusion protein comprising a Cas protein or peptide fused to a transcriptional repressor or activator. In some embodiments, the Cas protein is catalytically dead. The fusion protein may be directed to a site of interest in the genome by either an sgRNA or a crRNA. On binding of the fusion protein to the site of interest, the transcriptional repressor or activator can regulate the expression of a gene of interest.

Combination Therapies

Compounds of the present invention or identified by methods of the present invention may be used in the treatment of tumours and cancer in subjects in need of treatment thereof. The compounds may be administered alone or in combination with other anticancer agents.

An “anticancer agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository. Some chemotherapeutic agents are cytotoxic.

Cytotoxic chemotherapeutic agents trigger cell death via mechanisms or means that are not receptor mediated. Cytotoxic chemotherapeutic agents trigger cell death by interfering with functions that are necessary for cell division, metabolism, or cell survival. Because of this mechanism of action, cells that are growing rapidly (which means proliferating or dividing) or are active metabolically will be killed preferentially over cells that are not. The status of the different cells in the body as dividing or as using energy (which is metabolic activity to support function of the cell) determines the dose of the chemotherapeutic agent that triggers cell death. Cytotoxic chemotherapeutic agents non-exclusively relates to alkylating agents, anti-metabolites, plant alkaloids, topoisomerase inhibitors, antineoplastics and arsenic trioxide, carmustine, fludarabine, IDA ara-C, myalotang, GO, mustargen, cyclophosphamide, gemcitabine, bendamustine, total body irradiation, cytarabine, etoposide, melphalan, pentostatin and radiation.

Anticancer agents also include protein kinase inhibitors which can be used in the treatment of a diverse range of cancers, including blood and lung cancers. Protein kinases typically promote cell proliferation, survival and migration and are often constitutively overexpressed or active in cancer. Inhibitors of protein kinases are therefore a common drug target in the treatment of cancers. Examples of kinase inhibitors for use in the clinic include Crizotinib, Ceritinib, Alectinib, Brigatinib, Bosutinib, Dasatinib, Imatinib, Nilotinib, Ponatinib, Vemurafenib, Dabrafenib, Ibrutinib, Palbociclib, Sorafenib, and Ribociclib.

Anticancer agents also include agents for use in immunotherapy, including antibodies. Immunotherapies can elicit, amplify, reduce or suppress an immune response depending on the specific disease context. For example, tumour cells expressing the PDL1 ligand suppress the normal immune response in a subject by binding to PD-1 receptor expressed on T cells. In this way, tumour cells resist immunity-induced apoptosis and promote tumour progression. Anti-PD-1 and anti-PDL1 antibodies have been employed successfully in the clinic to inhibit this immune checkpoint and promote immune cell-mediated killing of tumour cells. Other examples of immunotherapy Include oncolytic viral therapies, T-cell therapies, and cancer vaccines.

Pharmaceutical Compostions

The pharmaceutical compositions provided herein may include one or more pharmaceutically acceptable excipients, e.g., solvents, solubility enhancers, suspending agents, buffering agents, isotonicity agents, antioxidants or antimicrobial preservatives. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans. When used, the excipients of the compositions will not adversely affect the stability, bioavailability, safety, and/or efficacy of the active ingredients. Thus, the skilled person will appreciate that compositions are provided wherein there is no incompatibility between any of the components of the dosage form. Excipients may be selected from the group consisting of buffering agents, tonicity agents, chelating agents, antioxidants, antimicrobial agents, and preservatives.

Expression of Therapeutic Products

The nucleic acid that is delivered by the compositions of the invention may exhibit a therapeutic action (e.g. by acting directly to down or up regulate a target gene) or it may express a gene product (which could be a therapeutic protein or therapeutic nucleic acid) via an expression cassette comprising a coding sequence operably linked to a promoter. In this specification the term “operably linked” may include the situation where a selected nucleotide sequence and regulatory nucleotide sequence are covalently linked in such a way as to place the expression of a coding sequence under the influence or control of the regulatory sequence. Thus a regulatory sequence is operably linked to a selected nucleotide sequence if the regulatory sequence is capable of effecting transcription of a coding sequence which forms part or all of the selected nucleotide sequence. Where appropriate, the resulting transcript may then be translated into a desired protein or polypeptide.

Route of Administration

The compositions according to aspects of the present invention may be formulated for administration by a number of routes, including but not limited to, intravenous, parenteral, intra-arterial, intramuscular, intratumoural, subcutaneous, oral and nasal. The compositions may be administrated by injection to a human or animal subject, e.g. via intravenous, Intra-arterial, Intramuscular, Intradermal, subcutaneous or intratumoural injection.

Subject

The subject to be treated may be any animal or human. The subject is preferably mammalian, more preferably human. The subject may be a non-human mammal, but is more preferably human. The subject may be male or female. The subject may be a patient. Therapeutic uses may be in human or animals (veterinary use).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps. It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” It will be understood that the particular value forms another embodiment. The term “about” in relation to a numerical value is optional and means for example +/−10%.

### EXAMPLES

### Example 1-Gene Delivery with Peptide Dendrimers Displaying Cationic and Hydrophobic Residues in Combination with Lipids

### Introduction

Nucleic acid delivery systems based on cationic lipids are one of the most studied and efficient non-viral vector platforms described to date, and the rational design and development of peptidic vectors with natural amino acids are particularly attractive for therapeutic applications due to the non-toxic nature of the amino acids. The inventors have developed a structural framework for nucleic acid delivery, using peptide dendrimers. The structural framework involves three layers of peptide (or dipeptide) motifs, bound to lysine residues. The inventors have found that the distribution of cationic amino acid residues (Lys or Arg) In each generation (layer) gave peptide dendrimers transfecting more efficiently than dendrimers with charges localized solely on the surface (Kwok et al, 2013). Using a solid phase peptide dendrimer synthetic procedure, the inventors can precisely manipulate the position of every amino acid residue incorporated within the dendritic scaffold. This allows greater control of the structure and function of the dendrimer, which was normally not possible with previously studied systems such as polymers or other dendrimers where modifications were mainly made on the surface of the molecule. The peptide dendrimer/lipid vector showed high transfection efficiency, good reproducibility of results and low toxicity.

The inventors hypothesised that (1) the dendritic skeleton, (2) dendrimer compactness, (3) dendritic size, (4) dendrimer building block chirality and (5) the amino acid composition within the dendrimers may be important parameters for improving gene transfer. The inventors explored structural features by generating a new dendrimer library based on the effective G1,2,3-KL with alternating LysLeu motif and found that the transfection activity is generation dependent, with 3 generations being optimal for transfection. Attaining an even charge distribution in each generation is important, however the exact position of the charge within each generation is not important. Some activity was observed when using dendrimers of differing chirality and those having differing numbers of amino acids within each generation. However, changing the cationic amino acid from Lys to Arg within the dendrimer (i.e. from the LysLeu to ArgLeu motif) improved transfection in serum conditions. This would allow in vivo gene transfer via systemic delivery, which is correlated to the improved stability of the dendrimer-DNA complexes. The G1,2,3-RL complexes are effective in gene delivery to a panel of tissues.

Results

The Role of the Molecular Recognition Units in Determining Efficacy of Nucleic Acid Delivery

To assess the effect on fine tuning the molecular recognition units of the dendrimers on DNA packaging and delivery while keeping the balance of hydrophobic and cationic properties constant, the inventors investigated a dendrimers with different (1) skeleton, (2) compactness, (3) size, (4) chirality in the building block, and (5) amino acid composition, based on derivatives of G2,2,3-KL. The dendrimer-DNA complexes with DOTMA/DOPE (D/D). Transfection efficiency at the widely used human cervical cancer HeLa and mouse neuroblastoma Neuro2A cells was evaluated using certain dendrimers from the table below:

TABLE 1
Exemplary dendrimers. G1,2,3-KL; G1,2-KL; G1-KL; G1,2,3-LR; G1-LR, G2,3-
RL; G1,2,3-rl; G1,2-RL, G3-LR; G1,2-LR, G3-RL; G1,2,3-RL; G1,2,-RL; G1-
RL; G1,2-LK, G3-KL; G1,2,3-LK; Ala-G1,2,3-KL; G1,2,3-K; G0,1,2,3-KL; and
D-G1,2,3-KL (also denoted “G1,2,3-kl”) have alternating charges (Lys or
Arg) and hydrophobic (Leu) residues distribution throughout the dendrimer. G1,2,3-K
has just a single amino acid in the peptide motif of each generation. The
NH₂ groups present at the C terminus of the core sequences are a result
of the peptide synthesis method. The molecular weight (MW) does not include
the counter ion (trifluoroacetate).

| Name | Dendrimer structure | MW | Charge | MW/charge |
|---|---|---|---|---|
| G1,2,3-KL | (KL)₈(KKL)₄(KKL)₂KGSC-NH₂ | 4540.13 | +22 | 206 |
| G1,2-KL | (KL)₄(KKL)₂KGSC-NH₂ | 2096.80 | +10 | 210 |
| G1-KL | (KL)₂KGSC-NH₂ | 875.13 | +4 | 219 |
| G1,2,3-LR | (LR)₈(KLR)₄(KLR)₂KGSC-NH₂ | 4932.31 | +22 | 224 |
| G1-LR,G2,3-RL | (RL)₈(KRL)₄(KLR)₂KGSC-NH₂ | 4932.31 | +22 | 224 |
| G1,2,3-rl | (rl)₈(krl)₄(krl)₂kgsc-NH₂ | 4932.31 | +22 | 224 |
| G1,2-RL,G3-LR | (LR)₈(KRL)₄(KRL)₂KGSC-NH₂ | 4932.31 | +22 | 224 |
| G1,2-LR,G3-RL | (RL)₈(KLR)₄(KLR)₂KGSC-NH₂ | 4932.31 | +22 | 224 |
| G1,2,3-RL | (RL)₈(KRL)₄(KRL)₂KGSC-NH₂ | 4932.31 | +22 | 224 |
| G1,2,-RL | (RL)₄(KRL)₂KGSC-NH₂ | 2264.88 | +10 | 226 |
| G1-RL | (RL)₂KGSC-NH₂ | 931.16 | +4 | 233 |
| G1,2-LK,G3-KL | (KL)₈(KLK)₄(KLK)₂KGSC-NH₂ | 4540.13 | +22 | 206 |
| G1,2,3-LK | (LK)₈(KLK)₄(KLK)₂KGSC-NH₂ | 4540.13 | +22 | 206 |
| Ala-G1,2,3-KL | (KL)₈(KKL)₄(KKL)₂KGSA-NH₂ | 4508 | +22 | 206 |
| G1,2,3-K | (K)₈(KK)₄(KK)₂KGSC-NH₂ | 2955.92 | +22 | 134 |
| G0,1,2,3-KL | (KL)₈(KKL)₄(KKL)₂KKLC-NH₂ | 4637.33 | +23 | 202 |
| D-G1,2,3KL | (kl)₈(kkl)₄(kkl)₂kgsc-NH₂ | 4540.13 | +22 | 206 |

In vivo and in vitro transfection efficiency for mRNA and DNA delivery was also evaluated using certain dendrimers from the table below and discussed in the following examples:

TABLE 2
Further exemplary dendrimers. The NH2 groups present at the C terminus of the core sequences are
a result of the peptide synthesis method. X = 6-aminohexanoic acid, B = beta-alanine,
Dab = 2,4-Diaminobutyric acid. Lowercase letters refer to D-form amino acids, whereas uppercase
letters refers to L-form amino acids. l is the D-form leucine. There is no D or L form for glycine.

| Name | Dendrimer structure | MW |
|---|---|---|
| G1,2,3-KL | (KL)₈(KKL)₄(KKL)₂KGSC-NH₂ | 4540.13 |
| G1,2-KL | (KL)₄(KKL)₂KGSC-NH₂ | 2096.80 |
| G1-KL | (KL)₂KGSC-NH₂ | 875.13 |
| G1,2,3-LR | (LR)₈(KLR)₄(KLR)₂KGSC-NH₂ | 4932.31 |
| G1-LR,G2,3-RL | (RL)₈(KRL)₄(KLR)₂KGSC-NH₂ | 4932.31 |
| G1,2,3-rl | (rl)₈(krl)₄(krl)₂kgsc-NH₂ | 4932.31 |
| G1,2-RL,G3-LR | (LR)₈(KRL)₄(KRL)₂KGSC-NH₂ | 4932.31 |
| G1,2-LR,G3-RL | (RL)₈(KLR)₄(KLR)₂KGSC-NH₂ | 4932.31 |
| G1,2,3-RL | (RL)₈(KRL)₄(KRL)₂KGSC-NH₂ | 4932.31 |
| G1,2,-RL | (RL)₄(KRL)₂KGSC-NH₂ | 2264.88 |
| G1-RL | (RL)₂KGSC-NH₂ | 931.16 |
| G1,2-LK,G3-KL | (KL)₈(KLK)₄(KLK)₂KGSC-NH₂ | 4540.13 |
| G1,2,3-LK | (LK)₈(KLK)₄(KLK)₂KGSC-NH₂ | 4540.13 |
| Ala-G1,2,3-KL | (KL)₈(KKL)₄(KKL)₂KGSA-NH₂ | 4508 |
| G1,2,3-K | (K)₈(KK)₄(KK)₂KGSC-NH₂ | 2955.92 |
| G0,1,2,3-KL | (KL)₈(KKL)₄(KKL)₂KKLC-NH₂ | 4637.33 |
| D-G1,2,3KL | (kl)₈(kkl)₄(kkl)₂kgsc-NH₂ | 4540.13 |
| RHCG1-R | (R)2KRHC-NH2 | 854.06 |
| RHCG1,2-R | (R)4(KR)2KRHC-NH2 | 1735.16 |
| RHCG1,2,3-R | (R)8(KR)4(KR)2KRHC-NH2 | 3497.36 |
| RHCG1-LR | (LR)2KRHC-NH2 | 1080.38 |
| RHCG1-RL, 2-LR | (LR)4(KRL)2KRHC-NH2 | 2414.12 |
| RHCG1,2-RL,3-LR | (LR)8(KRL)4(KRL)2KRHC-NH2 | 5081.59 |
| KAG1,2-RL, 3-LR | (LR)8(KRL)4(KRL)2KA-NH2 | 4756.19 |
| RFWG1,2-RL, 3-LR | (LR)8(KRL)4(KRL)2KRFW-NH2 | 5174.69 |
| RYMG1,2-RL, 3-LR | (LR)8(KRL)4(KRL)2KRYM-NH2 | 5135.68 |
| YMG1,2-RL, 3-LR | (LR)8(KRL)4(KRL)2KYM-NH2 | 4979.49 |
| LinearG1,2-RL, 3-LR | (LR)8(KRL)4(KRL)2KGSCRHCRFWRYM-NH2 | 6269.00 |
| RHCG1-RLR | (RLR)2KRHC-NH2 | 1392.76 |
| RHCG1,2-RLR | (RLR)4(KRLR)2KRHC-NH2 | 3351.24 |
| RHCG1,2,3-RLR | (RLR)8(KRLR)4(KRLR)2KRHC-NH2 | 7268.21 |
| G1-LRLR | (LRLR)2KGSC-NH2 | 1469.88 |
| G1,2-LRLR | (LRLR)4(KLRLR)2KGSC-NH2 | 3881.00 |
| G1,2,3-LRLR | (LRLR)8(KLRLR)4(KLRLR)2KGSC-NH2 | 8703.24 |
| RHCG1-RLR, 2-RL, 3-R | (R)8(KRL)4(KRLR)2KRHC-NH2 | 4488.69 |
| RHCG1-RLR, 2-R, 3-LR | (LR)8(KR)4(KRLR)2KRHC-NH2 | 4941.33 |
| RHCG1-RLRL, 2-R, 3-LR | (LR)8(KR)4(KRLRL)2KRHC-NH2 | 5167.64 |
| G1-KL, 2-KH, 3-HK | (HK)8(KKH)4(KKL)2KGSC-NH2 | 4827.99 |
| G1-RE, 2-RL, 3-LR | (LR)8(KRL)4(KRE)2KGSC-NH2 | 4964.30 |
| G1-RF, 2-RM, 3-WR | (WR)8(KRM)4(KRF)2KGSC-NH2 | 5657.01 |
| G1-RX, 2-RB, 3-BR | (BR)8(KRB)4(KRX)2KGSC-NH2 | 4427.33 |
| G1-RQ, 2-RT, 3-YR | (YR)8(KRT)4(KRQ)2KGSC-NH2 | 5314.25 |
| G1-RW, 2-RQ, 3-FR | (FR)8(KRQ)4(KRW)2KGSC-NH2 | 5410.52 |
| G1-RY, 2-RT, 3-QR | (QR)8(KRT)4(KRY)2KGSC-NH2 | 5103.98 |
| G1-RC, 2-RC, 3-MR | (MR)8(KRC)4(KRC)2KGSC-NH2 | 5016.62 |
| G1-LL, 2-RR | (RR)4(KLL)2KGSC-NH2 | 2350.97 |
| D-G1,2-rl, 3-lr | (lr)8(krl)4(krl)2kgsc-NH2 | 4932.39 |
| LK-G1,2-rl, 3-lr | (lr)8(Krl)4(Krl)2Kgsc-NH2 | 4932.39 |
| G1-Rl, 2-rL, 3-lR | (lR)8(KrL)4(kRl)2KGSC-NH2 | 4932.39 |
| DabG1,2-RL, 3-LR | (LR)8(DabRL)4(DabRL)2DabGSC-NH2 | 4736.36 |
| G1-LR,2,3-RL | (RL)8(KRL)4(KLR)2KGSC-NH2 | 4932.39 |
| G1,2-RL,3-LR | (LR)8(KRL)4(KRL)2KGSC-NH2 | 4932.39 |
| G1,2-LR,3-RL | (RL)8(KLR)4(KLR)2KGSC-NH2 | 4932.39 |
| G1,2,3-LR | (LR)8(KLR)4(KLR)2KGSC-NH2 | 4932.39 |
| NTX1 | (LR)8(KRL)4(KRL)2KGSCGAASSLNIAXRXRRBRRXRRBRXB-NH2 | 7632.43 |
| NTX2 | (LR)8(KRL)4(KRL)2KGSCGAASSLNIA(Acp)-NH2 | 5830.37 |

The hydrodynamic size, polydispersity index and Zeta potential was determined for a set of the dendrimers disclosed in Tables 1 and 2.

TABLE 3
The hydrodynamic size and zeta potential of the peptide dendrimer,
lipid and mRNA complexes. The dendrimers were at an N:P ratio
of 0.16:1 and the lipids, DOTMA/DOPE, were at a w/w ratio of
10:1 to mRNA. The NH2 groups present at the C terminus of the
core sequences are a result of the peptide synthesis method.
X = 6-aminohexanoic acid, B = beta-alanine, Dab =
2,4-Diaminobutyric acid. Lowercase letters refer to D-form amino
acids, whereas uppercase letters refers to L-form amino acids.
l is the D-form leucine. There is no D or L form for glycine.

| | Hydrodynamic | Polydispersity | Zeta |
|---|---|---|---|
| | size | Index | Potential |
| Name | (nm) | (PDI) | (mV) |
| RHCG1-R | 129 | 0.123 | 33 |
| RHCG1,2-R | 123 | 0.114 | 37 |
| RHCG1,2,3-R | 125 | 0.173 | 43 |
| RHCG1-LR | 129 | 0.107 | 40 |
| RHCG1-RL, 2-LR | 117 | 0.155 | 41 |
| RHCG1,2-RL,3-LR | 134 | 0.11 | 44 |
| KAG1,2-RL, 3-LR | 125 | 0.136 | 47 |
| RFWG1,2-RL, 3-LR | 120 | 0.152 | 39 |
| RYMG1,2-RL, 3-LR | 133 | 0.137 | 43 |
| YMG1,2-RL, 3-LR | 117 | 0.116 | 35 |
| LinearG1,2-RL, 3-LR | 118 | 0.143 | 42 |
| RHCG1-RLR | 105 | 0.363 | 41 |
| RHCG1,2-RLR | 116 | 0.154 | 39 |
| RHCG1,2,3-RLR | 130 | 0.154 | 42 |
| G1-LRLR | 122 | 0.264 | 40 |
| G1,2-LRLR | 130 | 0.181 | 41 |
| G1,2,3-LRLR | 130 | 0.133 | 45 |
| RHCG1-RLR, 2-RL, 3-R | 119 | 0.219 | 36 |
| RHCG1-RLR, 2-R, 3-LR | 121 | 0.136 | 41 |
| RHCG1-RLRL, 2-R, 3-LR | 135 | 0.131 | 43 |
| G1-KL, 2-KH, 3-HK | 131 | 0.237 | 35 |
| G1-RE, 2-RL, 3-LR | 134 | 0.096 | 45 |
| G1-RF, 2-RM, 3-WR | 142 | 0.125 | 41 |
| G1-RX, 2-RB, 3-BR | 130 | 0.108 | 41 |
| G1-RQ, 2-RT, 3-YR | 130 | 0.135 | 41 |
| G1-RW, 2-RQ, 3-FR | 133 | 0.146 | 37 |
| G1-RY, 2-RT, 3-QR | 126 | 0.16 | 42 |
| G1-RC, 2-RC, 3-MR | 121 | 0.148 | 43 |
| G1-LL, 2-RR | 135 | 0.124 | 41 |
| D-G1,2-rl, 3-lr | 134 | 0.148 | 43 |
| LK-G1,2-rl, 3-lr | 132 | 0.16 | 45 |
| G1-Rl, 2-rL, 3-lR | 135 | 0.22 | 42 |
| DabG1,2-RL, 3-LR | 124 | 0.117 | 34 |
| G1,2,3-RL | 129 | 0.106 | 41 |
| G1-LR,2,3-RL | 132 | 0.136 | 41 |
| G1,2,3-rl | 130 | 0.095 | 41 |
| G1,2-RL, 3-LR | 127 | 0.122 | 16 |
| G1,2-LR,3-RL | 137 | 0.144 | 32 |
| G1,2,3-LR | 121 | 0.168 | 43 |
| NTX1 | 128 | 0.13 | 37 |

TABLE 4
The hydrodynamic size and zeta potential of the peptide dendrimer,
lipid and mRNA complexes. The dendrimers were at an N:P ratio of
8:1 and the lipids, DOTMA/DOPE, were at a w/w ratio of 10:1 to mRNA.
The NH₂ groups present at the C terminus of the core sequences
are a result of the peptide synthesis method. X = 6-aminohexanoic
acid, B = beta-alanine, Dab = 2,4-Diaminobutyric acid.
Lowercase letters refer to D-form amino acids, whereas uppercase
letters refers to L-form amino acids. l is the D-form leucine.
There is no D or L form for glycine.

| | Hydrodynamic | Polydispersity | Zeta |
|---|---|---|---|
| | size | Index | Potential |
| Name | (nm) | (PDI) | (mV) |
| RHCG1-R | 366 | 0.566 | 45 |
| RHCG1,2-R | 172 | 0.252 | 49 |
| RHCG1,2,3-R | 70 | 0.396 | 62 |
| RHCG1-LR | 326 | 0.577 | 35 |
| RHCG1-RL, 2-LR | 206 | 0.109 | 46 |
| RHCG1,2-RL,3-LR | 58 | 0.363 | 50 |
| KAG1,2-RL, 3-LR | 76 | 0.184 | 51 |
| RFWG1,2-RL, 3-LR | 91 | 0.401 | 55 |
| RYMG1,2-RL, 3-LR | 68 | 0.257 | 56 |
| YMG1,2-RL, 3-LR | 73 | 0.213 | 56 |
| LinearG1,2-RL, 3-LR | 62 | 0.289 | 54 |
| RHCG1-RLR | 183 | 0.766 | |
| RHCG1,2-RLR | 83 | 0.214 | |
| RHCG1,2,3-RLR | 73 | 0.299 | 56 |
| G1-LRLR | 250 | 0.581 | 49 |
| G1,2-LRLR | 92 | 0.189 | 53 |
| G1,2,3-LRLR | 80 | 0.287 | 50 |
| RHCG1-RLR, 2-RL, 3-R | 83 | 0.429 | 59 |
| RHCG1-RLR, 2-R, 3-LR | 75 | 0.439 | 59 |
| RHCG1-RLRL, 2-R, 3-LR | 78 | 0.37 | 56 |
| G1-KL, 2-KH, 3-HK | 103 | 0.126 | 49 |
| G1-RE, 2-RL, 3-LR | 96 | 0.158 | 53 |
| G1-RF, 2-RM, 3-WR | 80 | 0.277 | 51 |
| G1-RX, 2-RB, 3-BR | 70 | 0.403 | 56 |
| G1-RQ, 2-RT, 3-YR | 100 | 0.409 | 54 |
| G1-RW, 2-RQ, 3-FR | 105 | 0.452 | 51 |
| G1-RY, 2-RT, 3-QR | 95 | 0.178 | 52 |
| G1-RC, 2-RC, 3-MR | 87 | 0.19 | 56 |
| G1-LL, 2-RR | 213 | 0.087 | 47 |
| D-G1,2-rl, 3-lr | 73 | 0.246 | 53 |
| LK-G1,2-rl, 3-lr | 74 | 0.251 | 53 |
| G1-Rl, 2-rL, 3-lR | 74 | 0.241 | 56 |
| DabG1,2-RL, 3-LR | 78 | 0.286 | 57 |
| G1,2,3-RL | 163 | 0.509 | 41 |
| G1-LR,2,3-RL | 80 | 0.417 | 53 |
| G1,2,3-rl | 83 | 0.21 | 53 |
| G1,2-RL, 3-LR | 84 | 0.261 | 56 |
| G1,2-LR,3-RL | 89 | 0.242 | 54 |
| G1,2,3-LR | 83 | 0.272 | 60 |

Variation of the Dendritic Structure, Symmetry, Topology and Chirality

Various dendritic structures provide different amino acid distributions within the scaffolds and the number of amino acids between branching points to give structures displaying diverse compactness and size (e.g., see Table 1, above).

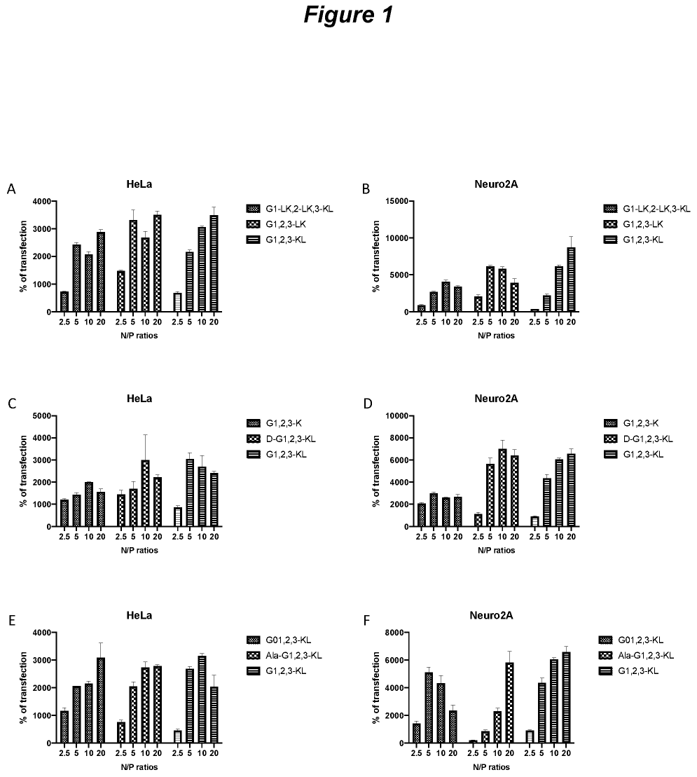

To investigate the effect of the topological variations, the dendrimers with the position of some or all KL reversed (G1,2-LK,3-KL and G1,2,3-LK) are compared with G1,2,3-KL. G1,2,3-LK did not have a significant difference on DNA binding and transfection compared to G1,2,3-KL (FIG. 1A,B). For G1,2,3-K, in which the total charge is the same as G1,2,3-KL but the size and molecular weight are reduced (the MW per charge for G1,2,3-K is 1.5 fold lower than G1,2,3-KL), transfection efficacy is reduced somewhat; not as effective as G1,2,3-KL (FIG. 1C,D). There is a trend that G1,2,3-K transfected better at a lower NIP ratio of 2.5 while increasing the N/P ratios made it less effective when compared to G1,2,3-KL.

To investigate the effect of chirality, a G1,2,3-KL dendrimer with D-form amino acids was synthesized (denoted D-G1,2,3KL). Replacing the L-form amino acid to the D-form of the dendrimers did not have any significant impact on transfection (FIG. 1C,D).

The importance of the amino acid sequence at the focal point on transfection was studied by testing G0,1,2,3-KL and Ala-G1,2,3-KL. The GlySerCys sequence in the focal point, being constant in all dendrimers previously studied, was replaced by the LysLeuAla with an additional charged residue. The change did not affect the transfection results (FIG. 1E,F).

Overall, the above results indicate that the charge distribution within each generation of the dendrimer is the determining factor for transfection efficacy. Further variations on an optimal structure may change the transfection activity slightly, but the overall effect is not important, provided that the charge distribution per se is not changed. This observation is in line with our previous observations that charge only concentrated in the outer generations, but not evenly distributed across each generation, resulted in a much decreased transfection efficiency.

Comparison of the Transfection Efficiency of KL and RL Dendrimers with Different Generations

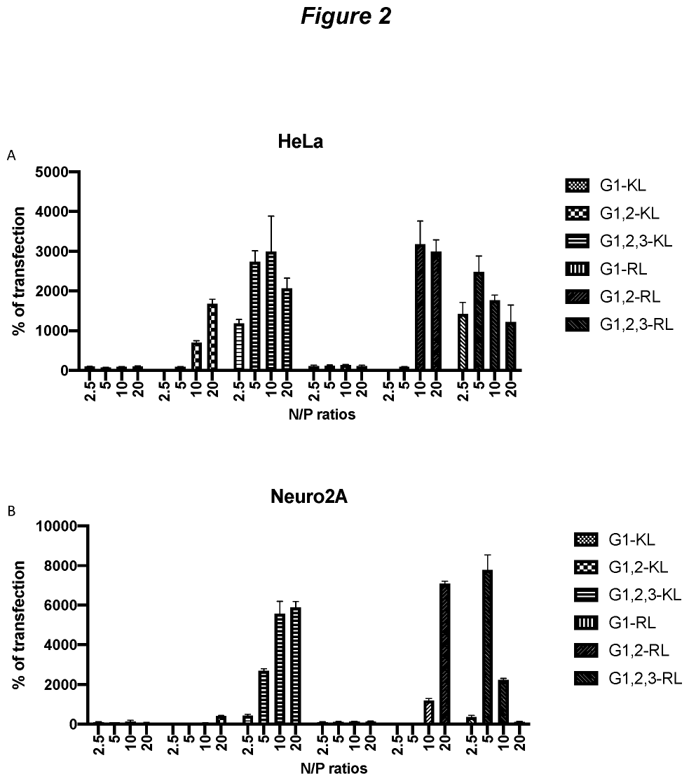

The number of generations from G1 to G2 to G3 based on the KL repeating unit in delivery efficiency was explored. The KL unit was also replaced with the RL repeating unit to compare the effect of protonated basic group with different pKa. Interestingly we observed a relationship between generations and transfection in the cells (FIG. 2). The generation dependence on transfection reveals that first generation dendrimers G1-KL or G1-RL do not transfect HeLa or Neuro2A cells with lysine or arginine as charged residues. This can be due to the fact that G1 peptides are not physically large enough to fully encapsulate plasmid DNA sufficiently to form a stable nanoparticle, as shown by the complex stability assays (data not shown).

For second generation however the arginine containing G1,2-RL shows a higher transfection efficiency (at an N/P ratio of 10 or 20 In HeLa cells and at an N/P ratio of 20 In Neuro2A cells) in comparison to G1,2-KL (FIG. 2). Such transfection superiority of G1,2-RL to G1,2-KL is in line with the ability of G1,2-RL to form more stable transfection complexes than G1,2-KL with DNA (data not shown). The optimal NIP ratio of G2 dendrimers is significantly higher than the optimal N/P ratio of G3 dendrimers for the respective best activity. Our results also showed that G1,2-KL transfected HeLa cells in a higher efficiency than in Neuro2A cells. This highlights the fact that the activity of certain transfectants can be cell dependent due to different components such as proteoglycans, etc. and their concentration on the cell surface which could have an impact on transfection complex binding and ultimately gene delivery to cells.

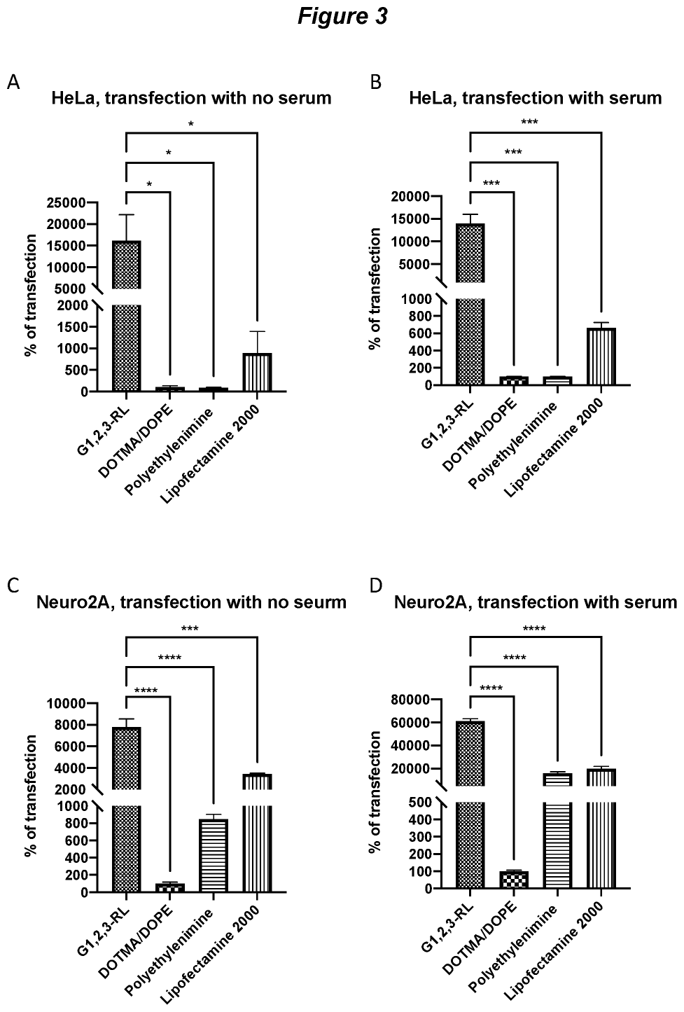

Overall, the G3 dendrimers transfect efficiently in both HeLa and Neuro2A cells. Our third generation dendrimers transfected 2-600 times better than some of the widely used commercial reagents such as Polyethylenimine (PEI), Lipofectin (also known as DOTMA/DOPE) and Lipofectamine 2000 in HeLa and Neuro2A cells (FIG. 3).

In the presence of serum, the G1,2,3-RL-dendrimer/DOTMA/DOPE formulations were up to 800 fold more efficient than DOTMA/DOPE alone (with transfection using D/D alone is defined at 100%). G1,2,3-KL gave a 200 fold improvement of transfection efficiency compared to D/D alone in HeLa cells (FIG. 4A). In the Neuro 2A cells, the activity of G1,2,3-KL and G1,2,3-RL were 340 and 1500 fold higher than DOTMA/DOPE alone respectively (FIG. 4B). This observation can be explained by the fact that the RL dendrimers bind DNA more strongly than the KL dendrimer and hence they are more stable and resistance to the challenge from the charged components in the serum.

The cytotoxicity mediated by the transfection reagents is an important parameter for in vivo applications. Toxicity of the dendrimer containing formulations was compared with that of controls. Most of the modifications of the dendrimers did not induce higher toxicity to the HeLa cells, with cell viability comparable to G1,2,3-KL (˜40% or higher) and the commercial reagent Lipofectamine 2000 (˜20%), and similar to the supplement lipid (D/D, ˜40%). Increasing the number of generation from 1 to 3 did not increase toxicity. The actual mechanism of the toxicity caused by the dendrimer formulations is not well known, however, part of the toxicity from the formulations was due to the addition of the D/D as shown before and also from the D/D alone control. Indeed, most of the lipid-dendrimer-DNA complexes did not mediate a higher toxic compared to the D/D control, while most of the dendrimer complexes resulted in higher cell viability compared to the widely used lipofectamine 2000. The toxicity of the dendrimer complexes was not directly related to the transfection efficiency as the ineffective G1-KL or RL formulations also induced 40% of cell death. In Neuro2A cells, most dendrimer-DNA complexes were not as toxic (80% or higher cell viability) as they were in the HeLa cells. Overall, the cell viability following transfection was significantly higher than the reference reagent Lipofectamine 2000 (˜45%).

Uptake Mechanism

The uptake mechanism of the composition of the invention was investigated. Chlorpromazine was used to inhibit clathrin mediated endocytosis. Genistein and Rottlerin were also used; these agents have been reported to inhibit caveolae-mediated endocytosis and micropinocytosis respectively.

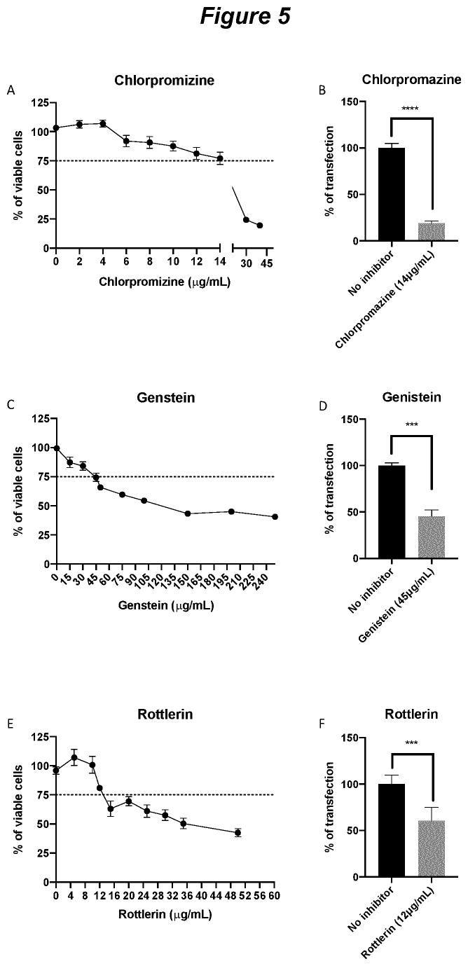

The inventors observed that the addition of Chlorpromazine significantly inhibited transfection by 80% (FIG. 5B), suggesting clathrin-mediated endocytosis plays a significant role in DNA delivery by the dendrimer system. Genistein and Rottlerin inhibited transfection by around 50% (FIG. 5D,F). This suggested that the peptide dendrimer system mediates cellular uptake via at least three different pathways, with clathrin-mediated endocytosis as the key pathway for internalisation. The ability to trigger different cellular internalisation pathways would allow uptake of the nanoparticles by different cell types and hence would explain the fact that this dendrimer system delivers the DNA cargo in vivo.

### DISCUSSION

The inventors have previously reported the effect of charge distribution of the RL and KL (i.e. a combination of a hydrophobic and a cationic group) based third generation dendrimers on transfection (Kwok et al, 2013). Providing an even distribution over the three generations gave efficient transfection reagents (in combination with DOTMA/DOPE) whereas concentration of charges only on the surface (third generation) reduced DNA transfection significantly. The present investigation on third generation dendrimer variants disclosed herein (e.g. at table 1, above) shows that, for efficient transfection, charge distribution in the dendritic structure is more important than the topology and chirality of amino acids within the scaffold.

The inventors demonstrate that the number of generations is important for transfection, with a trend that increasing the number of generations enhances transfection efficiency. The improved transfection efficiency is in line with improved complex stability. For instance, G1,2,3-RL is a highly effective dendrimer for transfection in serum condition and in vivo gene delivery (Data not shown).

Through systemic administration, the G1,2,3-RL, DOTMA/DOPE complex delivers DNA for functional gene expression in all the tissues assayed. Gene expression was especially high in liver and skeletal muscle without toxicity being observed. Delivering DNA to all different tissues is in line with the observation that the G1,2,3-RL and DOTMA/DOPE complexes can mediate cellular internalisation via clathrin-mediated endocytosis, caveolae-mediated endocytosis and macropinocytosis, which are cargo uptake process expressed in all cell types at different levels (data not shown).

The biodistribution of the G1,2,3-RL, DOTMA/DOPE, DNA composition differs from other systems described in the field. For instance, it was observed that lipid based, peptide based systems and dendrimer PAMAM scaffolds mainly delivered DNA to lung and spleen following an IV administration. PEI and amphiphilic peptide delivered DNA effectively to lung and liver, although toxicity was observed with the use of PEI. In contrast, the present invention provides an effective means of delivering to muscle, without toxicity. The relatively low gene expression in kidney and spleen implies that the composition of the invention is not subject to the clearance by the renal and reticuloendothelial systems. High gene expression in the liver therefore suggests delivery to mainly hepatocytes and stellate (data not shown).

The composition of the invention can also deliver nucleic acids functionally to the brain. While the route through or around the blood brain barrier (BBB) is not certain, transcytosis, a process that the endothelial cells endocytose the cargo at one end and exocytose at the other end of the barrier is a possibility. Transcytosis has been reported to deliver cargos bypassing the BBB by other synthetic systems such the bolaamphiphile system.

Surprisingly, the composition of the invention delivers DNA effectively to skeletal muscles, despite the fact that most non-viral gene delivery systems show ineffective delivery to this target (data not shown). Skeletal muscles exclusively express caveolin 3 (Cav3), which may suggest that the delivery system of the invention interacts with the Cav3 component to mediate effective cellular internalisation.

The peptidic dendrimers of the invention represent a versatile platform, providing efficient and potentially target-tailored transfection reagents with a large chemical space to be available with respect to the amino acid composition, the topology of the dendrimers and possible modifications on the C and N termini. The peptide dendrimer/DOTMA/DOPE system can bypass the biological barriers to deliver in vivo the DNA plasmid to the cell nucleus without any other adjuvant. It is envisaged that cell specific targeting domains, peptides or other molecules to help endosome escape and/or nuclear localization can be conjugated to the dendrimers to further improve cell/tissue targeting delivery and gene expression.

Materials and Methods

G1,2,3-KL ((KL)₈(KKL)₄(KKL)₂KGSC-NH₂) was obtained as foamy colourless solid after preparative RP-HPLC (72.3 mg, 10.3 μmol, 9%). Analytical RP-HPLC: t_{R}=1.47 min (A/D 100/0 to 0/100 in 2.2 min, λ=214 nm). MS (ESI+): C₂₁₈H₄₂₂N₆₀O₃₉S calc/found 4540.1/4539.0 [M]⁺.

G1,2-KL ((KL)₄(KKL)₂KGSC-NH₂) was obtained as foamy colourless solid after preparative RP-HPLC (90.5 mg, 27.9 μmol, 41%). Analytical RP-HPLC: t_{R}=1.36 min (A/D 100/0 to 0/100 in 2.2 min, λ=214 nm). MS (ESI+): C₉₆H₁₈₂N₂₈O₁₉S calc/found 2096.8/2096.0 [M]⁺.

G1-KL ((KL)₂KGSC-NH₂) was obtained as foamy colourless solid after preparative RP-HPLC (46.0 mg, 41.7 μmol, 60%). Analytical RP-HPLC: t_{R}=1.23 min (A/D 100/0 to 0/100 in 2.2 min, λ=214 nm). MS (ESI+): C₃₉H₇₄N₁₂O₉S calc/found 875.1/875.4 [M]⁺.

G1,2,3-RL ((RL)₈(KRL)₄(KRL)₂KGSC-NH₂) was obtained as foamy colourless solid after preparative RP-HPLC (14.6 mg, 2.0 μmol, 2%). Analytical RP-HPLC: t_{R}=1.50 min (A/D 100/0 to 0/100 in 2.2 min, λ=214 nm). HRMS (NSI+): C₂₁₈H₄₂₂N₈₈O₃₉S calc/found 4932.3/4931.4 [M]⁺; 5106.3/5105.7 [M+TFA+Na+K]+; 5220.3/5219.9 [M+2 TFA+Na+K]⁺; 5616.4/5616.3 [M+6 TFA]⁺; 5730.4/5730.3 [M+7 TFA]⁺; 5844.5/5844.3 [M+8 TFA]⁺; 5958.5/5958.3 [M+9 TFA]⁺; 6072.5/6072.3 [M+10 TFA]⁺.

G1,2-RL ((RL)₄(KRL)₂KGSC-NH₂) was obtained as foamy colourless solid after preparative RP-HPLC (143.2 mg, 42.1 μmol, 38%). Analytical RP-HPLC: t_{R}=1.45 min (AD 100/0 to 0/100 in 2.2 min, λ=214 nm). MS (ESI+): C₉₆H₁₉₀N₄₀O₁₉S calc/found 2264.9/2264.0 [M]⁺.

G1-RL ((RL)₂KGSC-NH₂) was obtained as foamy colourless solid after preparative RP-HPLC (78.5 mg, 56.6 μmol, 51%). Analytical RP-HPLC: t_{R}=1.37 min (A/D 100/0 to 0/100 in 2.2 min, λ=214 nm). MS (ESI+): C₃₈H₇₄N₁₆O₉S calc/found 931.2/931.2 [M]⁺; 1045.2/1045.4 [M+1 TFA]⁺.

G1,2-LK,3-KL ((KL)₈(KLK)₄(KLK)₂KGSC-NH₂) was obtained as foamy colourless solid after preparative RP-HPLC (56.7 mg, 8.1 μmol, 7%). Analytical RP-HPLC: t_{R}=1.47 min (A/D 100/0 to 0/100 in 2.2 min, λ=214 nm). MS (ESI+): C₂₁₈H₄₂₂N₆₀O₃₉S calc/found 4540.1/4539.0 [M]⁺.

G1,2,3-LK ((LK)₈(KLK)₄(KLK)₂KGSC-NH₂) was obtained as foamy colourless solid after preparative RP-HPLC (32.4 mg, 4.6 μmol, 4%). Analytical RP-HPLC: t_{R}=1.41 min (A/D 100/0 to 0/100 In 2.2 min, λ=214 nm). MS (ESI+): C₂₁₈H₄₂₂N₆₀O₃₉S calc/found 4540.1/4539.0 [M]⁺.

Ala-G1,2,3-KL ((LysLeu)₈(LysLysLeu)₄(LysLysLeu)₂LysGlySerAla-NH₂ was obtained from TentaGel S RAM® resin (500 mg, 0.22 mmol g−1), Ala-G1,2,3-KL as foamy colorless solid after preparative RP-HPLC (36.6 mg, 5.2 μmol, 5%). Analytical RP-HPLC: tR=1.46 min (100% A to 100% D over 2.2 min, λ=214 nm); MS (ESI+): C₂₁₈H₄₂₂N₆₆O₃₉ found/calc 4508.0/4508.1 [M]⁺.

G1,2,3-K ((K)₈(KK)₄(KK)₂KGSC-NH₂) was obtained as foamy colourless solid after preparative RP-HPLC (79.9 mg, 14.6 μmol, 12%). Analytical RP-HPLC: t_{R}=1.17 min (A/D 100/0 to 0/100 in 2.2 min, λ=214 nm). MS (ESI+): C₁₃₄H₂₆₈N₄₆O₂₅S calc/found 2955.9/2956.0 [M]⁺.

G0,1,2,3-KL ((KL)₈(KKL)₄(KKL)₂KKLC-NH₂) was obtained as foamy colourless solid after preparative RP-HPLC (54.2 mg, 7.5 μmol, 6%). Analytical RP-HPLC: t_{R}=1.47 min (A/D 100/0 to 0/100 in 2.2 min, λ=214 nm). MS (ESI+): C₂₂₅H₄₃₇N₆₁O₃₈S calc/found 4637.3/4638.0 [M]⁺.

D-G1,2,3-KL ((kl)₈(kkl)₄(kkl)₂kgsc-NHz) was obtained as foamy colourless solid after preparative RP-HPLC (28.3 mg, 4.0 μmol, 5%). Analytical RP-HPLC: t_{R}=1.44 min (A/D 100/0 to 0/100 in 2.2 min, λ=214 nm). MS (ESI+): C₂₁₈H₄₂₂N₆₀O₃₉S calc/found 4540.1/4540.0 [M]⁺ containing small impurities

DNA Transfection

Cell Lines, transfection reagents and plasmids. HeLa cells were maintained in RPMI medium with 10% (v/v) FCS and 1% (v/v) P/S in a humidified atmosphere in 5% CO₂ and 37° C. The plasmid pCl-Luc was derived from plasmid pCl (Promega, Southampton, UK) with the luciferase gene inserted. Branched PEI (25 kDa) was purchased from Sigma-Aldrich. Lipofectamine 2000 (L2000) and Lipofectin (DOTMA:DOPE, 1:1 (w/w)) were obtained from Invitrogen. PEI, L2000 and lipofectin were used as positive control transfection agents in accordance with the manufacturer's instructions.

Transfection procedure. 24 hours before transfection, HeLa cells were seeded (10,000 cells in 100 μL/well) in 96 well plates in order to reach 70% confluence. Plasmid transfection complexes were formed by mixing the dendrimers (100 μL, from 60 μg to 105 μg, dependent on N/P ratios) with lipofectin (4 μg; 100 μL). These mixtures were incubated with a pCl-Luc (4 μg; 100 μL) at different N/P ratios in OptiMEM for 30 min at 25° C. Transfection control complexes (PEI, Lipofectin or L2000) (total 100 μL in OptiMEM) were mixed with pCl-Luc (4 μg; 100 μL) at the respective manufacturers' recommended concentrations. For transfection in serum-free medium, before overlaying the DNA complexes on the cells, OptiMEM was added to dilute the complexes, so that each complex contained 0.25 μg DNA in a total volume of 100 μL in one well of a 96 well plate. For transfection in serum medium, before overlaying the DNA complexes on the cells, full growth medium was added to dilute the complexes, so that each complex contained 0.25 μg DNA in a total volume of 100 μL in one well of a 96 well plate. After removing complete media from the cells, the complexes were added to the plates. The plates were incubated for 4 hours at 37° C. Then, the transfection solutions were replaced by full growth media for 24 hours before luciferase activity was assayed.

Transgene expression assay. The cells were washed twice with PBS and incubated with reporter lysis buffer (20 μL, Promega) for 20 min at 4° C., then overnight at −80° C. After the cells were defrosted at room temperature, luciferase assay buffer (100 μL, Promega; prepared according to the manufacturer's protocol) was added to each well. The luminescent product was measured by Relative Light Units (RLU) in a FLUOstar Optima luminometer (BMG Labtech).

Protein content determination. The protein content of each cell lysate was determined by mixing the lysate (20 μL) with Bio-Rad Protein Assay Reagent (180 μL, Bio-Rad, Hemel Hempstead, UK). After incubation of 10 min, the absorbance at 590 nm was measured and converted to protein concentration using a BSA standard curve. RLU per mg of protein represented luciferase activity. The ratio of these two values is the activity per protein unit (in RLU/mg). The values displayed in the transfection figures are represented after normalisation against a control transfection experiment with DOTMA/DOPE and are shown as percentages.

Cell viability. Cells were transfected as described in ‘Transfection Procedure’. Following 24 hours of transfection, the medium was removed and the cells were washed twice with PBS. Afterwards, the cells were dried for 1 hour at room temperature (to allow permeation of the nuclear stain). Crystal violet solution (50 μL of stock solution supplied by Sigma-Aldrich) was added to the cells. They were incubated for 15 min at room temperature. After washing with distilled water (5 times), the cells were dried for 30 min at room temperature. Then, MeOH (200 μL) was added and the suspension was incubated for 1 hour at room temperature. The relative amount of the crystal violet stain retained by viable cells was determined by the absorbance of the methanolic solution at 550 nm.

Fluorescence Quenching Assay

PicoGreen (Invitrogen) was added to DNA and diluted in TE buffer (10 mM Tris/HCl at pH 7.5; 1 mM EDTA) to a final DNA concentration of 0.002 μg/μL. PicoGreen was added to DNA in a ratio of 1:150 (v/v), so that every 100 μL of the solution contained 0.2 μg DNA. The mixture was incubated for 10 min at room temperature. During the incubation, different amounts of transfection reagents were diluted in TE buffer. Then, 50 μL of lipofectin (0.2 μg) and 50 μL of dendrimers (the amount depends on the chosen NIP ratio that was varied from 0.625:1 to 40:1) were added per well of flat-bottomed 96 well plates. Afterwards, 100 μL of DNA-PicoGreen solution (0.2 μg DNA) was added per well. As a control, 100 μL DNA-PicoGreen solution (0.2 μg DNA) was added to 100 μL of TE buffer. Following 30 min incubation at room temperature, 100 μL of TE buffer was added to each well. The PicoGreen signal was then detected with a fluorescent plate reader (FLUOstar Optima, BMG Labtech) with excitation at 485 nm and emission at 520 nm. The PicoGreen signals from the complexes were normalized against the DNA control to yield the percentage of the PicoGreen signal detected.

Complex Dissociation Assay

PicoGreen (Invitrogen) was added to DNA and diluted in TE buffer (10 mM Tris/HCl at pH 7.5; 1 mM EDTA) to a final DNA concentration of 0.002 μg/μL (PicoGreen was added to DNA in a ratio of 1:150 (v/v), so that every 100 μL of the solution contained 0.2 μg DNA). The mixture was incubated for 10 min at room temperature. During the incubation, different amounts of transfection reagents were diluted in TE buffer. Then, 50 μL of lipofectin (0.2 μg) and 50 μL of dendrimers (the amount depends on the chosen N/P ratio that was varied from 0.625:1 to 40:1) were added per well of flat-bottomed 96 well plates. Afterwards, 100 μL of DNA-PicoGreen solution (0.2 μg DNA) was added per well. As a control, 100 μL DNA-Picogreen solution (0.2 μg DNA) was added to 100 μL TE buffer. Following 30 min incubation at room temperature, different concentrations of heparin (0.2 to 1.4 U/mL, Sigma-Aldrich) diluted in 100 μL of TE buffer was added to DNA-complexes or DNA alone (0.2 μg, total volume 200 μL). After 30 min incubation at room temperature, the fluorescent signal from PicoGreen was recorded using a microplate reader (FLUOstar Optima, BMG Labtech) with excitation at 485 nm and emission at 520 nm. The DNA control was used to normalize the signal.

In Vivo Luciferase DNA Expression Assays

Mice were injected with the dendrimers, lipid and DNA formulations, and tissues were isolated and snap-frozen for luciferase expression analysis 24 h and 48 h post intravenous and intramuscular injection respectively.

The tissues were homogenised with 1× reporter lysis buffer supplemented with Protease Inhibitors. Depending on the tissues, 1.5 to 3 times of the lysis buffer was used for the tissue homogenisation. Following the homogenisation, the lysates were centrifuged and the supernatants were used for assaying luciferase expression and protein content. The luciferase expression level was measured with a luminometer using a luciferase assay kit (Promega), following the manufacturer's instructions. The protein content was measured using a Biorad protein assay kit at 595 nm absorbance, following the manufacturer's instructions.

In Vivo Luciferase mRNA Expression Assays

Peptide Dendrimer-Lipid-mRNA Particles

Peptide dendrimers were diluted to a respective concentration and mixed with mRNA for a desired NP ratio such as NP=0.15:1 or 8:1. The solution was incubated in room temperature for 10 mins. Lipid such as DOTMA/DOPE was diluted to a respective concentration and mixed with the dendrimer-mRNA complexes for a desired NP ratio such as NP-4.7:1. The solution was incubated in room temperature for 20 mins. When required, the solution would be further diluted before injection.

Lipid-mRNA Particle Control

DOTMA/DOPE was diluted to a respective concentration and mixed with the dendrimer-mRNA complexes. The solution was incubated in room temperature for 20 mins. When required, the solution would be further diluted before injection.

Mice and Delivery Route

All animal procedures conducted in our studies complied with UK laws and were inclusive of ethics approval. Female CD-1 mice (n=39) aged 6-8 weeks left to habituate for 1-week upon their arrival. All mice were weighed, and the formulations were delivered intravenously (100 μl) via the tail vein using 30G insulin syringes (BD biosciences).

Bioluminescence Imaging (BLI)

All mice were imaged 6 hours post injection. BLI was performed using an IVIS Lumina II (Perkin Elmer) Imaging system. Mice were administered D-luciferin (30 mg/mL, XenoLight, Perkin Elmer) at a dose of 150 mg/kg. Mice were anaesthetised 6 mins after receiving D-luciferin in a chamber with 5% isoflurane and then placed on a heated imaging platform while being maintained on 2.5% isoflurane. Mice were imaged 10 min post administration of D-luciferin with an exposure time set to ensure the signal acquired was within an effective detection range (open filter, binning 8, f-stop 1). Bioluminescence signal was quantified by measuring photon flux (photons/s) in the defined region of interest (ROI) using the Living Image software (Perkin Elmer). Following in vivo whole-body imaging, the mice were euthanised, cardiac blood taken, and the tissues extracted for ex vivo imaging. Here each tissue was placed into individual wells of a 24-well imaging plate (black sided, Eppendorf) containing 0.3 mg/mL D-luciferin in PBS). The imaging plate was placed in the centre of imaging platform (Lumina II system) and signal was measured using the acquisition settings detailed above. Finally, the tissues were placed in storage vials and flash frozen in liquid nitrogen.

Endocytic Experiments

Cells were preincubated with each inhibitor (chlorpromazine, genstein and rottlerin) for 30 min at 37 C.

Subsequently transfection was performed for a further 4 h at 37 C as described in the transfection section, in the presence of the inhibitor.

In Vivo Studies

Inducing FST Expression Using Compositions Comprising DNA

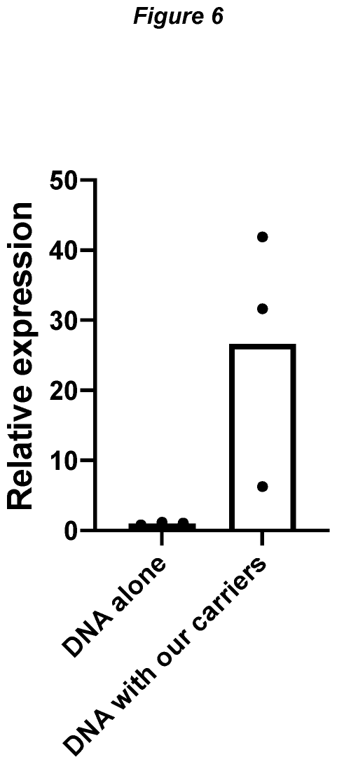

Mice were injected either with DNA expressing follistatin alone or DNA expressing follistatin with G1,2,3-RL with the D/D lipid on Day 1 and Day 3. On day 5, tissues were harvested for RNA extraction. cDNA was then synthesised from the mRNA for qPCR. The relative expression levels of follistatin (FST) in the skeletal muscle of the mice is presented in FIG. 6.

Immunogenicity/Tolerability

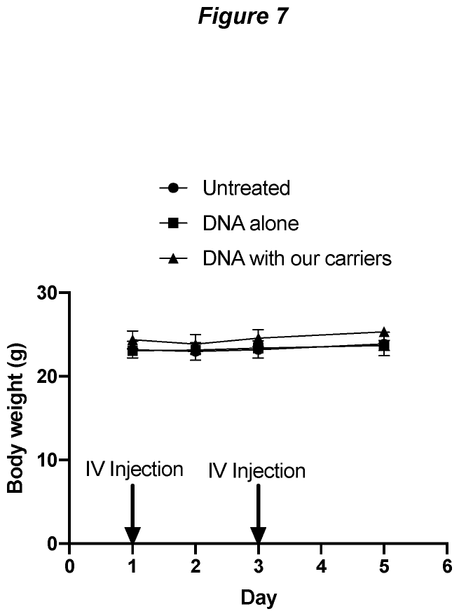

Body weight was monitored during IV protocols of the type for DNA delivery discussed above. No weight loss was observed (see FIG. 7).

Aspartate aminotransferase (AST) and Alanine aminotransferase (ALT) levels were also monitored, for mice that received no injection, or intravenous injection with DNA only or with the DNA, G1,2,3-RL and D/D formulation, on Day 1 and Day 3. AST levels remained substantially below 200 U/L and ALT levels remained below 100 U/L, for all groups. (These levels of AST and ALT are much lower than the threshold levels in disease model of liver damage (induced by carbon tetrachloride), of greater than 7000U/L. (Bonnet et al 2008))

Cytokine levels were also measured in the mice to check for immunogenicity. Serum was collected on Day 5 for the measurement of the AST and ALT level. No substantial elevations were observed. For IFN-gamma, the levels remained below 2 pg/mL, with no significant increase (other non-viral systems could elevate the IFN-gamma up to 60,000 pg/mL (Bonnet et al 2008)). For TNF-alpha, the levels remained below 20 pg/mL, with no significant increase (Lipopolysaccharides could increase the TNF-alpha up to 5000 μg/mL (Bonnet et al 2008)). For IL-6, the level for the group of mice administered with the composition of the invention remained below 20 pg/mL, with no significant increase (other non-viral systems could increase the IL-6 level up to 15000 pg/mL (Bonnet et al 2008)). For IL-1-beta and IL-10, the levels also remained very low, with no significant increase.

Inducing Luciferase Expression Using Compositions Comprising DNA

Mice were injected with the dendrimers, lipid and DNA formulation prepared for detection of luciferase expression post injection. Tissues were isolated and snap-frozen for luciferase expression analysis 24 h and 48 h post intravenous and intramuscular injection respectively.

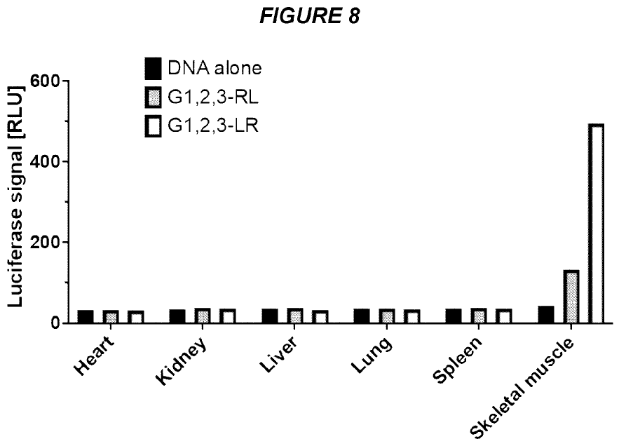

Compositions comprising both G1,2,3-RL and G1,2,3-LR injected intravenously targeted delivery of DNA to skeletal muscle as determined by luciferase expression, with G1,2,3-LR showing the most effective delivery to skeletal muscle (FIG. 8).

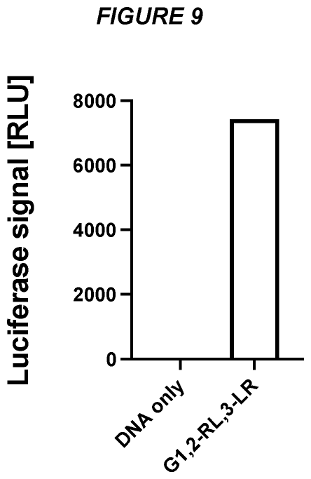

Compositions comprising G1,2-RL, 3-LR injected intramuscularly are also capable of delivering DNA to skeletal muscle as determined by luciferase expression (FIG. 9).

Inducing Luciferase Expression Using Compositions Comprising mRNA

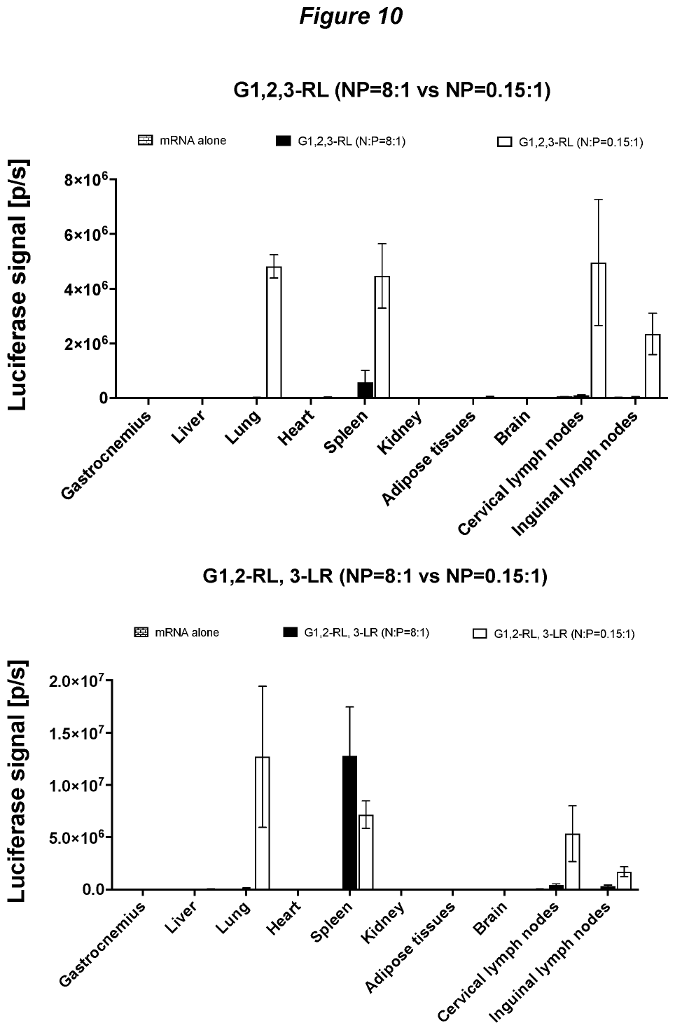

Mice were injected with a composition comprising DOTMA/DOPE, mRNA encoding luciferase and either G1,2,3-RL, (FIG. 10, top panel; the “G1,2,3-RL, lipid and mRNA formulation”) or G1,2-RL,3-LR (FIG. 10, bottom panel: the “G1,2,3-RL, lipid and mRNA formulation”). Each formulation was prepared and injected at an NP ratio of 8:1 and 0.15:1. Mice were prepared for detection of luciferase expression post injection.

Changing the NP ratio of either G1,2,3-RL or G1,2-RL,3-LR formulations leads to a change in the biodistribution of mRNA in mice tissues. An NP ratio of 0.15:1 targets a wide range of immune cell tissues including lung, spleen, and lymph nodes compared to an NP=8:1 which specifically targets spleens and to a lower extent the lymph nodes (FIG. 10). This demonstrates that altering the NP ratio of the composition can alter the tissue distribution of the composition. For instance, increasing the NP ratio can improve targeting to lymphoid organs.

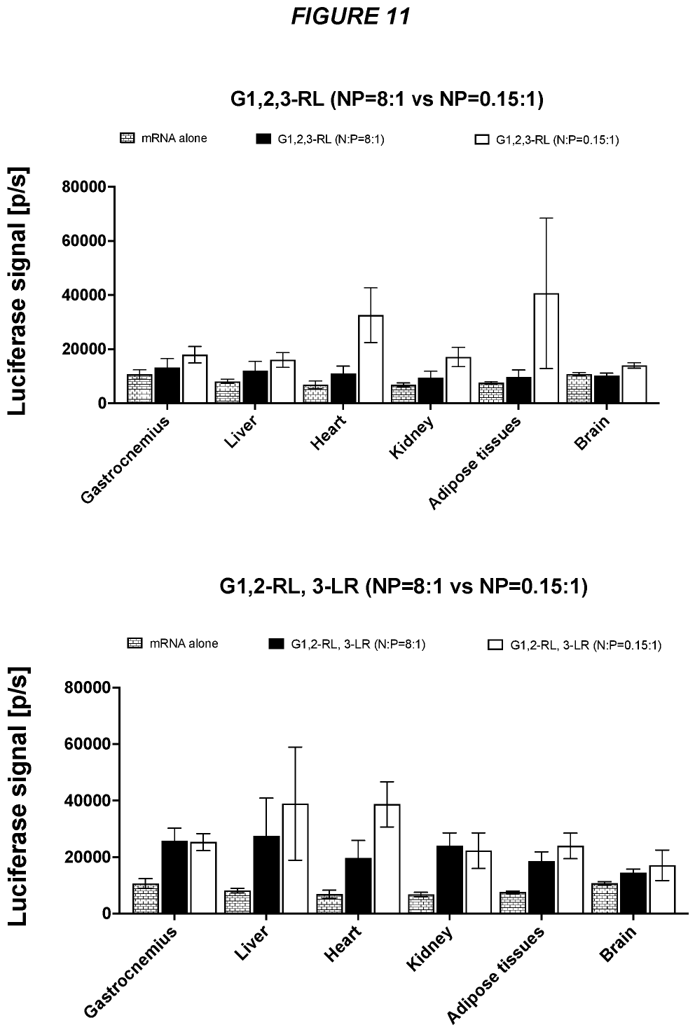

The G1,2,3-RL and G1,2-RL,3-LR, lipid and mRNA formulations can also successfully deliver mRNA to tissues such including muscle, liver, heart, kidney and adipose tissue (FIG. 11). In particular, the G1,2-RL, 3-LR, lipid and mRNA formulation, at an NP ratio of either 8:1 or 0.15:1 were able to induce significant expression in the Gastrocnemius and Quadricep muscle tissues, liver, heart, kidney and adipose tissue.

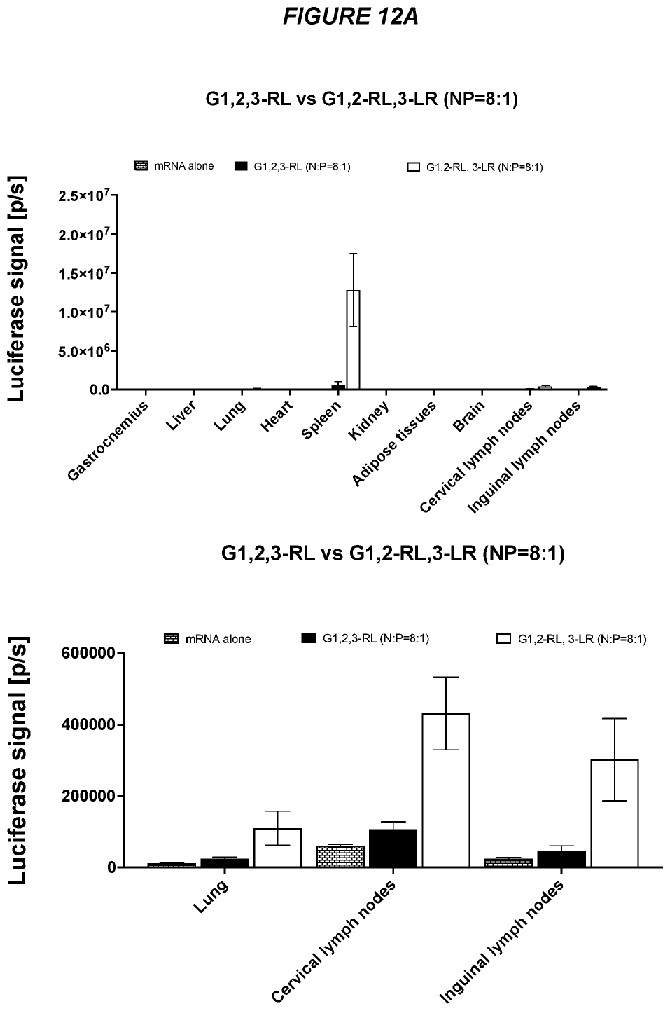

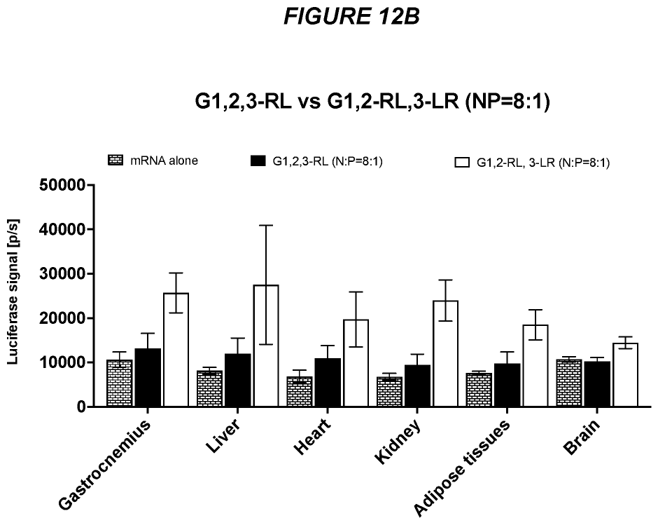

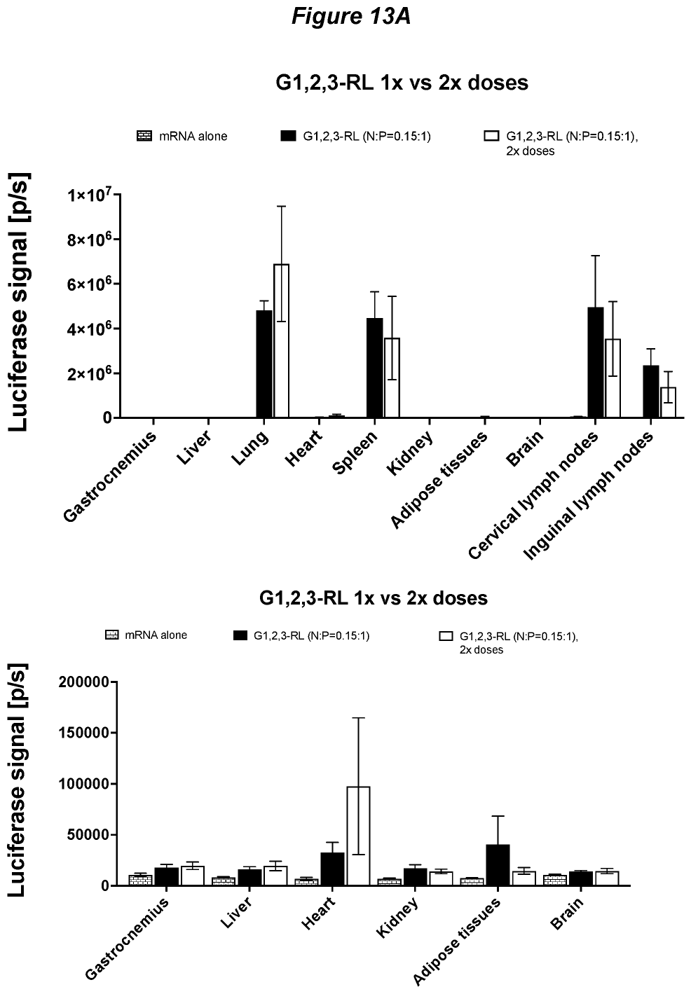

A comparison of luciferase expression was performed between mice injected with a composition comprising G1,2,3-RL, DOTMA/DOPE and mRNA vs mice injected with a composition comprising G1,2-RL,3-LR, DOTMA/DOPE and mRNA. Increased delivery to all tissues tested was observed for G1,2-RL,3-LR relative to G1,2,3-RL (FIG. 12). This demonstrates that the choice of dendrimer sequence can be used to target specific tissues.

Repeat Dosing Experiments

To determine if the compositions could be repeatedly dosed and maintain delivery to tissues, a comparison between mice receiving either a single or repeated dose (2 doses, 24 hours apart) of DOTMA/DOPE, mRNA and the peptide dendrimer was performed. Mice receiving two doses of either composition displayed increased luciferase expression compared to mRNA alone. Typically, luciferase expression in mice receiving two doses either displayed approximately the same degree of luciferase expression or increased luciferase expression compared to mice receiving a single dose. Luciferase expression was assayed 6 hours after treatment

Tissue/Cell Targeting Peptides-Dendrimer Fusion Proteins

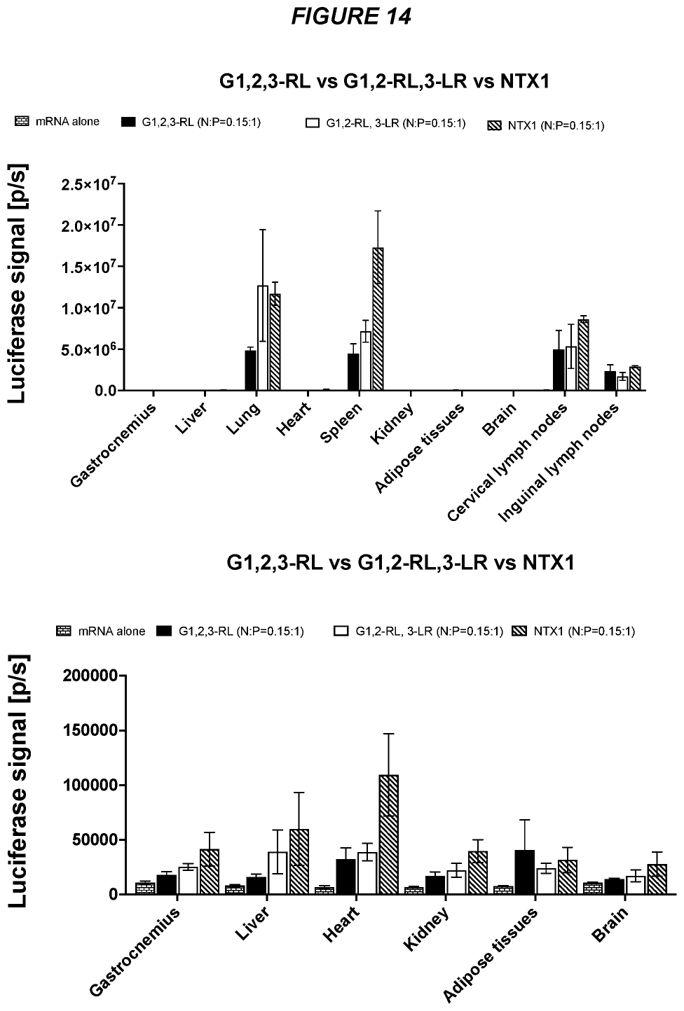

A comparison of luciferase expression was performed between compositions comprising DOTMA/DOPE, mRNA and one of G1,2,3-RL, G1,2-RL,3-LR, or NXT1 (FIG. 14). The NXT1 dendrimer comprises a G1,2-RL, 3-LR dendrimer with a core peptide sequence comprising a muscle targeting peptide and a cell penetrating peptide. NXT1 is more effective than G1,2,3-RL and G1,2-RL,3-LR at mediating mRNA delivery to a number of tissues, including muscle. Surprisingly, NXT1 is also more effective at delivering mRNA to spleen and lymph nodes compared to G1,2,3-RL and G1,2-RL,3-LR. NXT1 and G1,2-RL,3-LR show similar efficiency at delivering mRNA to the lungs.

Dendrimer Increases mRNA Delivery to Tissues Compared to Lipid Alone

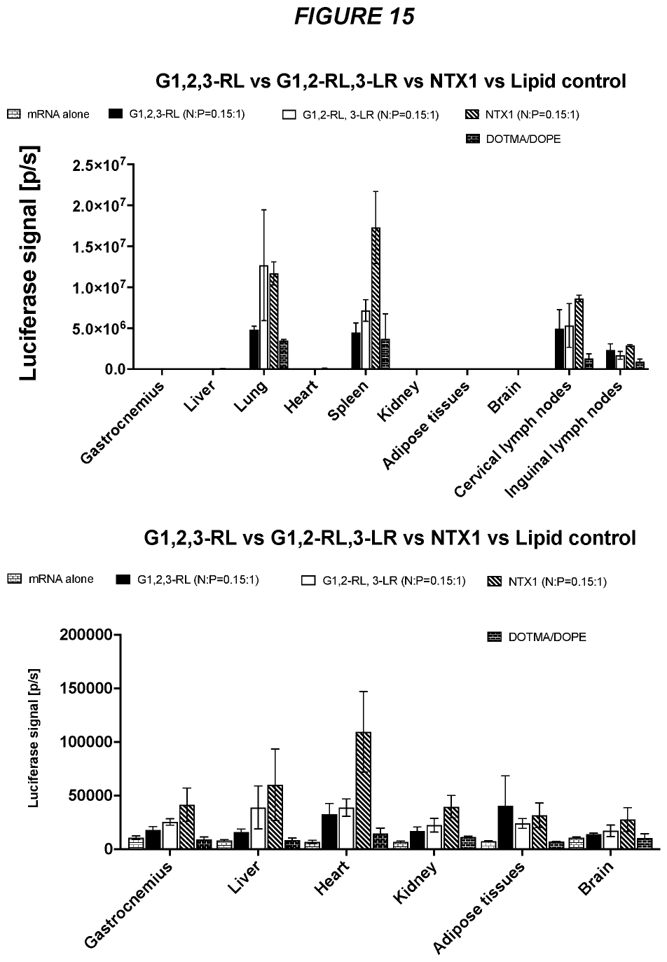

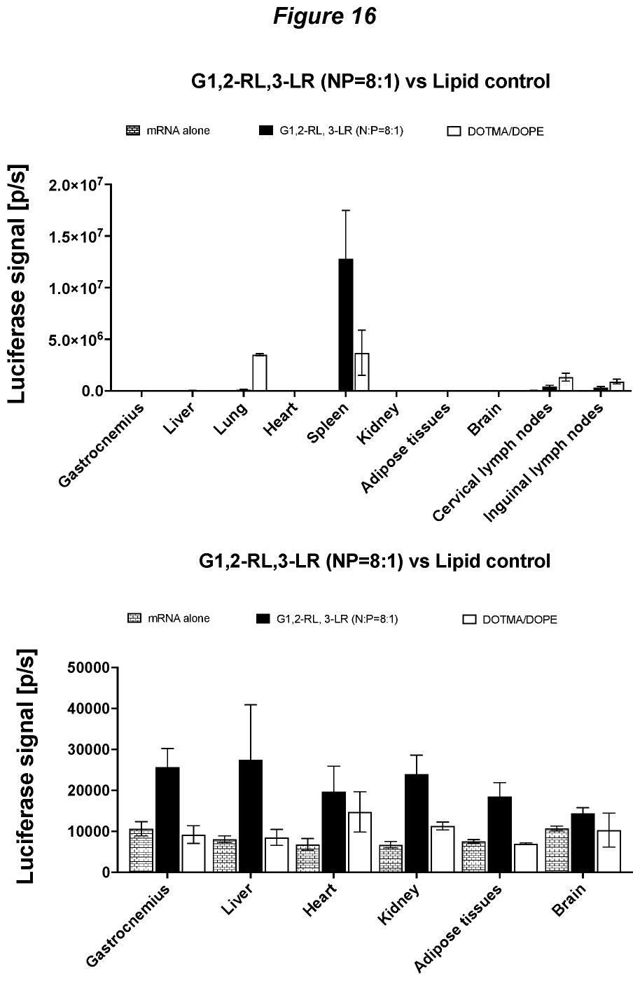

To determine whether the presence of dendrimers improves delivery of mRNA to tissues, mice were injected with compositions comprising either: i) mRNA alone; ii) mRNA and DOTMAJDOPE; iii) G1,2,3-RL, DOTMA/DOPE and mRNA; iv) G1,2-RL,3-LR, DOTMA/DOPE and mRNA, or v) NTX1, mRNA and DOTMA/DOPE and prepared for detection of luciferase expression post injection. The NP ratio in each dendrimer composition was 0.15:1. As demonstrated in FIG. 15, the presence of either G1,2,3-RL, G1,2-RL,3-LR and NTX1 improves efficiency of mRNA delivery to lung, spleen, and lymph nodes compared to mRNA alone or mRNA and DOTMA/DOPE alone.

A comparison was also performed between mice injected with either a composition comprising i) mRNA alone, li) mRNA, G1,2-RL, 3-LR and DOTMA/DOPE or III) mRNA and DOTMA/DOPE. The NP ratio in this experiment was 8:1. This demonstrates that mRNA delivery to the spleen is significantly enhanced in the presence of dendrimer compared to mRNA alone or mRNA and lipid. In addition, the expression of luciferase in lung and lymph nodes is decreased in compositions comprising dendrimer compared to lipid alone. This data further demonstrates that G1,2-RL,3-LR improves the specificity of mRNA delivery to the spleen compared to DOTA/DOPE alone.

Comparison of the Dendrimer; Lipid Delivery System to Commercially Available Lipid Delivery Systems

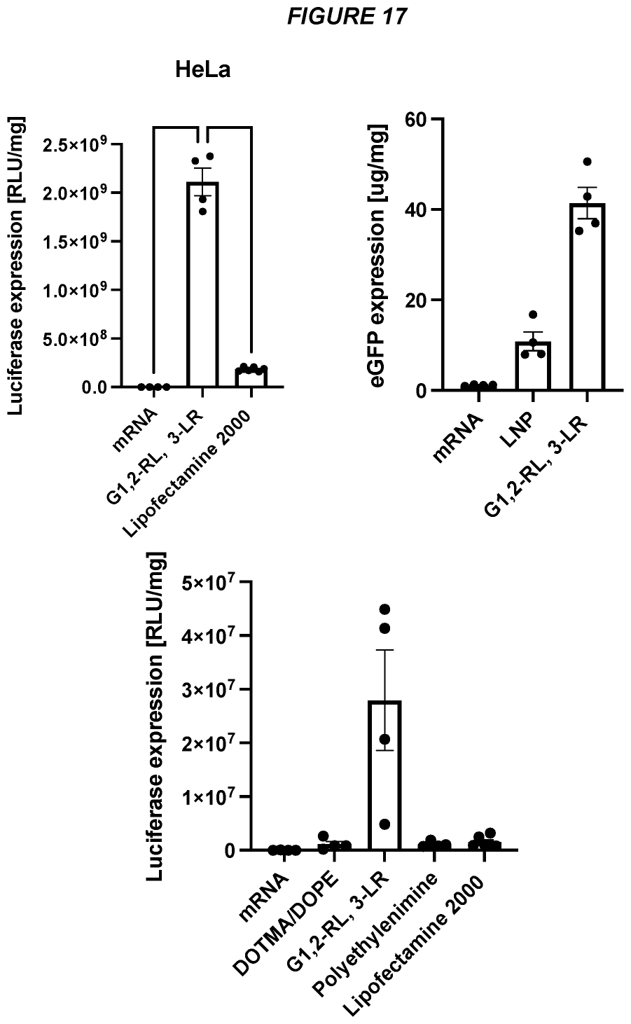

HeLa cells were transfected with mRNA encoding luciferase using either a composition comprising G1,2-RL, 3-LR and DOTMA/DOPE (NP=0.16:1) or commercially available Lipofectamine™ 2000. As shown in FIG. 17, top left panel, compositions comprising G1,2-RL, 3-LR with an NP ratio=0.16:1 and DOTMA/DOPE increase transfection efficiency in vitro by around an order of magnitude compared to the commercially available transfection reagent Lipofectamine™ 2000. Similarly, HeLa cells transfected with mRNA encoding eGFP using G1,2-RL, 3-LR at an NP ratio=0.16:1 and DOTMA/DOPE increased eGFP expression by approximately 4× compared to mRNA transfected using a DLin-MC3-DMA: Cholesterol: DSPC: DMG-PEG lipid nanoparticle delivery system (FIG. 17, top right panel).

C2C12 cells were transfected with either mRNA alone, G1,2-RL, 3-LR (N:P=8:1) and DOTMA/DOPE (at a w/w=10:1 to mRNA) with mRNA, DOTMA/DOPE (at a w/w=10:1 to mRNA) with mRNA, polyethylenimine with mRNA and Lipofectamine 2000 with mRNA for 24 hours. As shown in FIG. 17, bottom panel, dendrimer formulations improve delivery of mRNA to C2C12 cells compared to commercially available lipid-based transfection reagents.

In Vitro CRISPR Experiments

RNAs were labelled using Label IT® Nucleic Acid Labeling Kits (Mirus Bio LLC) according to the manufacturing instructions. Briefly, the single guide RNA (sgRNA), which consists of a CRISPR RNA (crRNA) fused with a tracerRNA (trRNA), was labelled with a TM-Rhodamine fluorophore while the Cas9 mRNA was labelled with a Cy5 fluorophore. crRNA and trRNA were tagged with a fluorescein and ATTO 550 fluorophore, respectively.

HeLa cells were transfected with different combinations of the labelled RNAs with a formulation of either (1) G1,2-RL, 3-LR at an NP ratio=0.15:1 and DOTMA/DOPE or (2) G1,2-RL, 3-LR at an NP=8:1 ratio and DOTMA/DOPE. The lipid to RNA ratio in all compositions was 10:1 w/w and DOTMA:DOPE was at a 1:1 weight ratio. Cells were incubated with each formulation for 2 hours and subsequently trypsinised and fixed. The percentage of the RNA uptake in the cells was assayed by flow cytometry. RNAs only without formulations were used as a control.

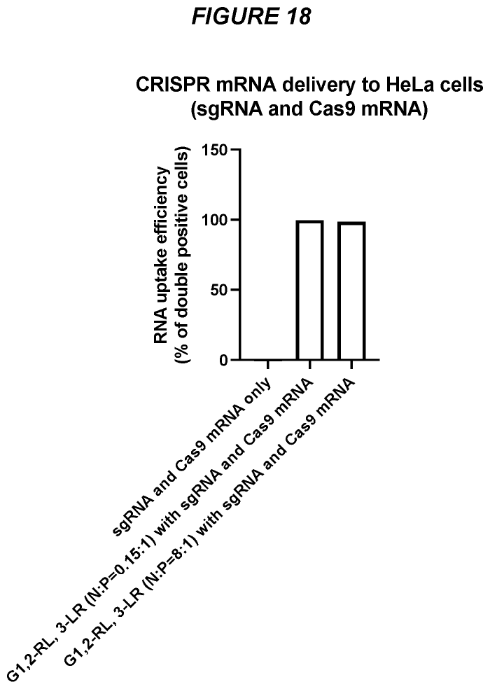

Approximately 100% of HeLa cells exposed to the sgRNA+Cas9 mRNA formulations were seen to be dual positive for sgRNA and Cas9 mRNA demonstrating that G1,2-RL, 3-LR dendrimers are highly effective transfection reagents (FIG. 18).

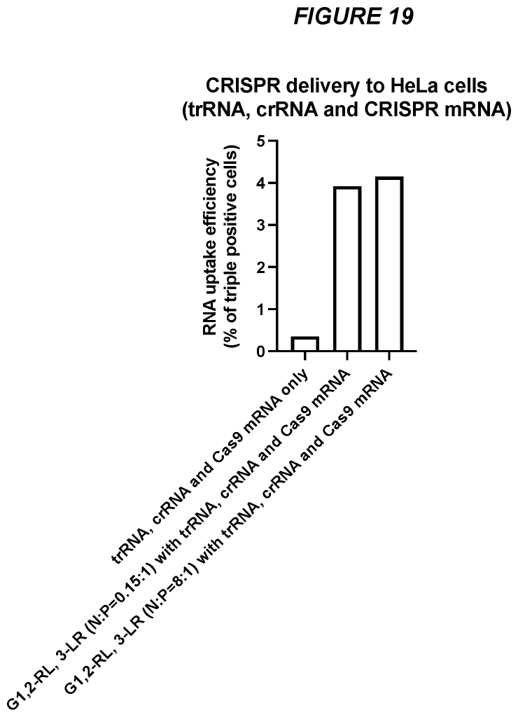

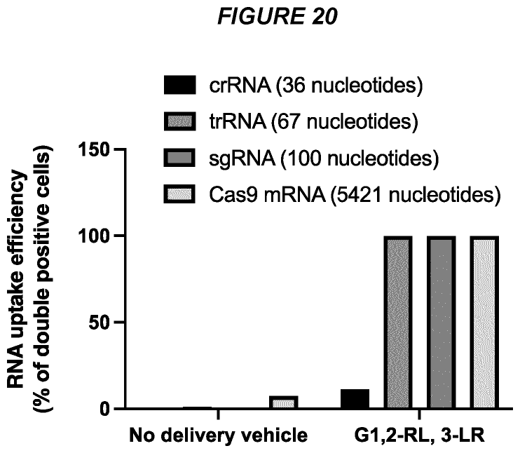

In dendrimer and lipid formulations comprising crRNA, trRNA and Cas9 mRNA, the dendrimer formulation can mediate delivery of all three components in around 4% of cells (FIG. 19). In dendrimer and lipid formulations comprising only one of the crRNA, trRNA or Cas9 mRNA, the percentage of positive cells varies. In composition comprising either trRNA or Cas9 mRNA, ˜100% of cells are positive for nucleic acid. In contrast, only 4% of cells are positive for crRNA when exposed to composition only comprising crRNA (FIG. 20). Together, this data demonstrates that while the G1, 2-RL, 3-LR dendrimer can successfully mediate delivery of relatively short RNAs (e.g. the crRNA used in this study was 36 nucleotides in length) to cells, the successful delivery of RNA increases as length of RNA to be transfected increases (trRNA and Cas9 mRNA used in this study was 67 and 5421 nucleotides in length, respectively).

Immune Cell Targeting

Fifteen 5-7-week-old female CD-1 mice (Charles River UK) were dosed with 1 mg/kg of either mRNA labelled with Alexa Fluor488 only or mRNA labelled with Alexa Fluor488 with NTX1 and DOTMA/DOPE or mRNA labelled with Alexa Fluor488 with G1,2-RL, 3-LR and DOTMA/DOPE or mRNA labelled with Alexa Fluor488 with G1,2-RL, 3-LR and DOTMA/DOPE, respectively, via intravenous injection using a dose volume of 5 ml/kg. Group 1 received vehicle and Group 5 received mRNA alone as controls. Mice in al groups were culled 2 hours post IV administration. At the time of euthanasia, whole blood, spleen, inguinal lymph nodes and femurs were collected from all mice. White Blood Cells (WBC) were obtained from EDTA treated whole blood, spleen, inguinal lymph nodes and bone marrow for flow cytometry analysis. In short, bone marrow (BM) was flushed from femurs, and spleens and lymph nodes were processed to single cell suspensions by passing them through a 70 μm filter. Red blood cells in blood, BM and spleen were lysed before isolated WBCs from all tissues were stained with different antibody panels to identify specific sub-populations of immune cells. The staining was performed in a 96-well plate format before samples were analysed using an ACEA Novocyte 3005 flow cytometer.

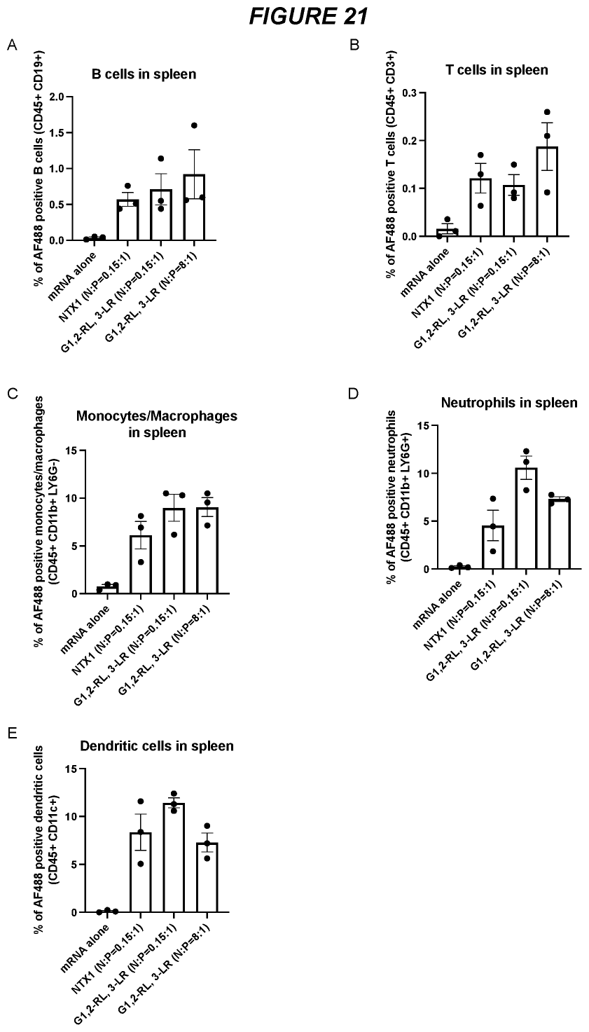

NTX1 and G1,2-RL, 3-LR mediated uptake of mRNA in immune cells present in the spleen (FIG. 21). mRNA formulated with dendrimers and lipids were taken up by (A) B cells, (B) T cells, (C) Monocytes and Macrophages, (D) Neutrophils and (E) dendritic cells. In particular, the dendrimer and lipid formulations can deliver mRNA to monocytes and macrophages, neutrophils, and dendritic cells within a relatively short time period from injection (2 hrs post-injection). NTX1 (NP=0.15:1) and G1,2-RL, 3LR (at an NP=0.15:1 and 8:1) mediates delivery of mRNA to ˜5% or more of monocytes/macrophages, neutrophils, and dendritic cells. Given the short time-period from injection to tissue harvest, it is reasonable to hypothesise that delivery of mRNA to each of the immune cell types analysed would increase over time.

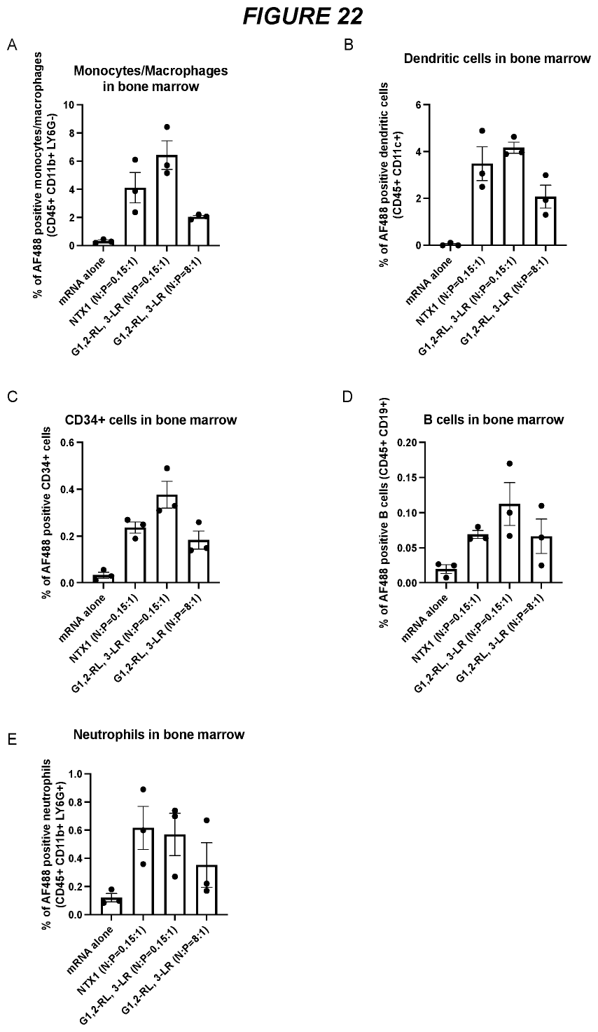

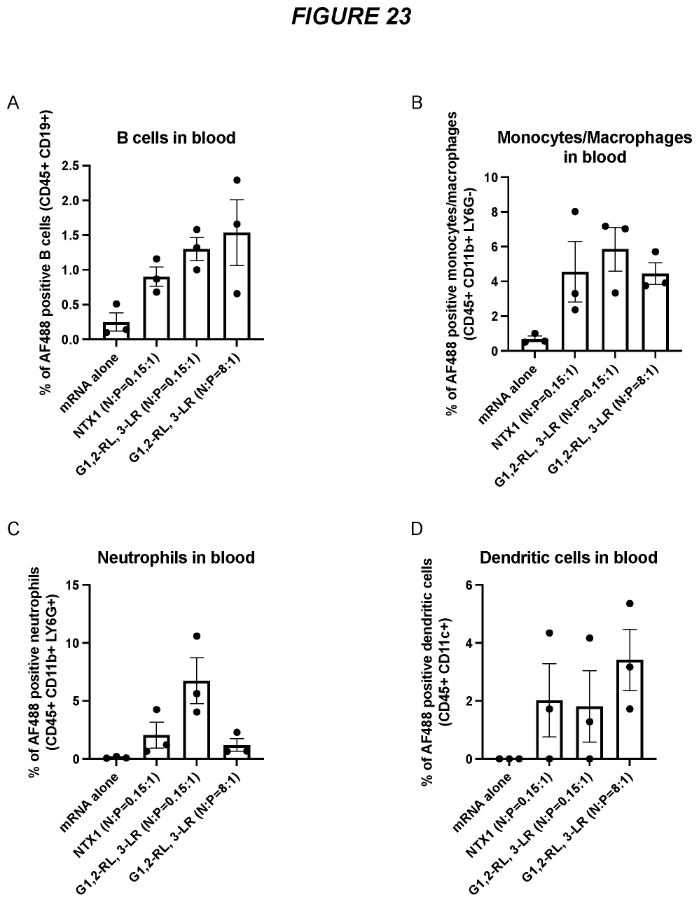

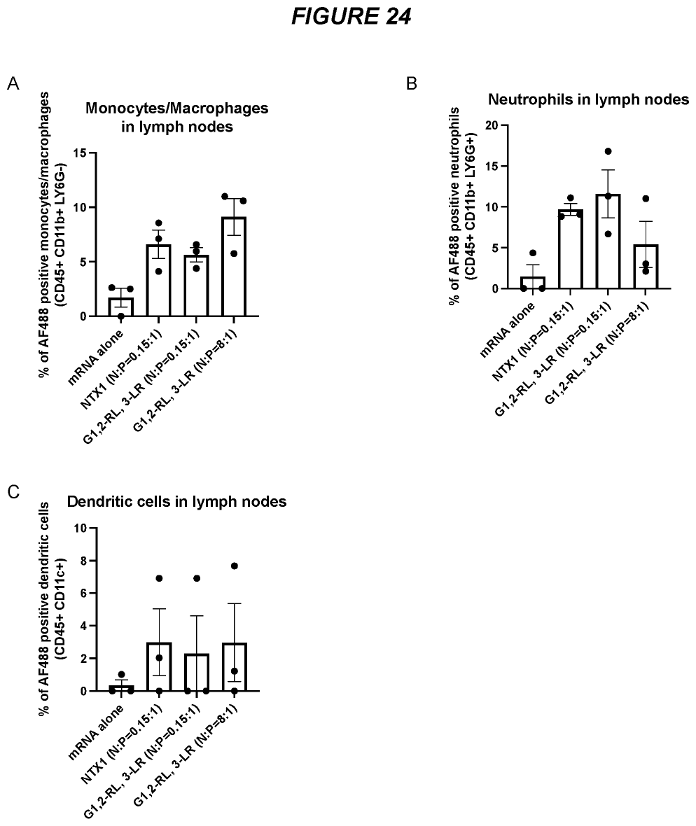

NTX1 and G1,2-RL, 3-LR mediated uptake of mRNA in immune cells present in the bone marrow (FIG. 22). mRNA formulated with dendrimers and lipids were taken up by (A) Monocytes and Macrophages (B) Dendritic cells (C) CD38+ cells, (D) B cells, and (E) Neutrophils. In particular, the dendrimer and lipid formulations can deliver mRNA to monocytes and macrophages, neutrophils, and dendritic cells within a relatively short time period from injection (2 hrs post-injection). Given the short time-period from injection to tissue harvest, it is reasonable to hypothesise that delivery of mRNA to each of the immune cell types analysed would increase over time.

In Vitro DNA Delivery

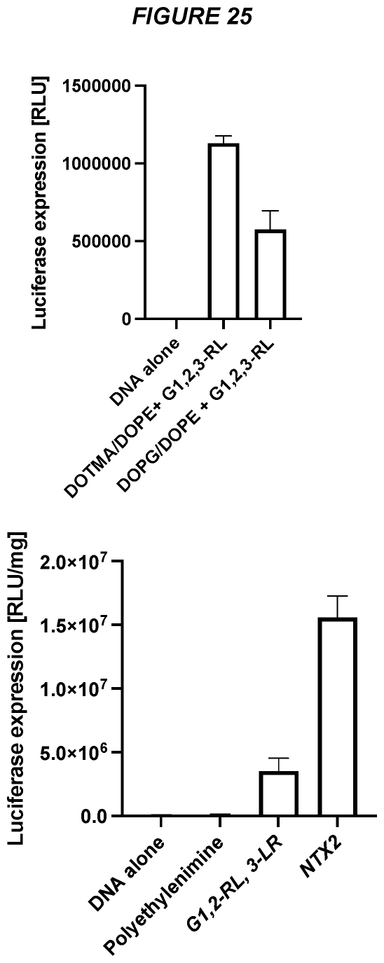

FIG. 25, top panel, shows luciferase expression in HeLa cells transfected with either DNA alone, G1,2,3-RL (N:P=5:1) and DOTMA/DOPE (at a w/w=1:1 to DNA) with DNA and G1,2,3-RL (N:P=5:1) and DOPG/DOPE (at a w/w=1:1 to DNA) with DNA. Compositions comprising G1,2,3-RL and either DOTMA/DOPE or DOPG/DOPE are capable of successfully transfecting HeLa cells in vitro.

Similarly, FIG. 25, bottom panel, shows luciferase expression in C2C12 cells (a muscle-derived cell line) transfected with either DNA alone, polyethylenimine with DNA, G1,2-RL, 3-LR (N:P=8:1) and DOTMA/DOPE (at a w/w=1:1 to DNA) with DNA and NTX2 (N:P=8:1) and DOTMA/DOPE (at a w/w=1:1 to DNA) with DNA. NTX2 is a G1,2-RL, 3-LR dendrimer conjugated to a muscle targeting peptide ASSLINA ((LR)8(KRL)4(KRL)2KGSCGAASSLNIA(Acp)-NH2). Both dendrimers are capable of successfully transfecting C2C12 cells in vitro. Dendrimers comprising a muscle targeting domain further increase transfection efficiency compared to dendrimers without a muscle targeting domain.

In Vivo Toxicity Assays

Mice were injected with the mRNA formulations either 1 or 2 times. For 2 IV injections, the mice were injected 24 hours prior to the 2nd injections. 6 hours after the 2nd IV injection, the plasma of the mice were harvested and the AST level and other cytokine levels were measured. For mice injected 1 time, plasma was harvested 6 hours post injection for the AST level and other cytokine levels measurement

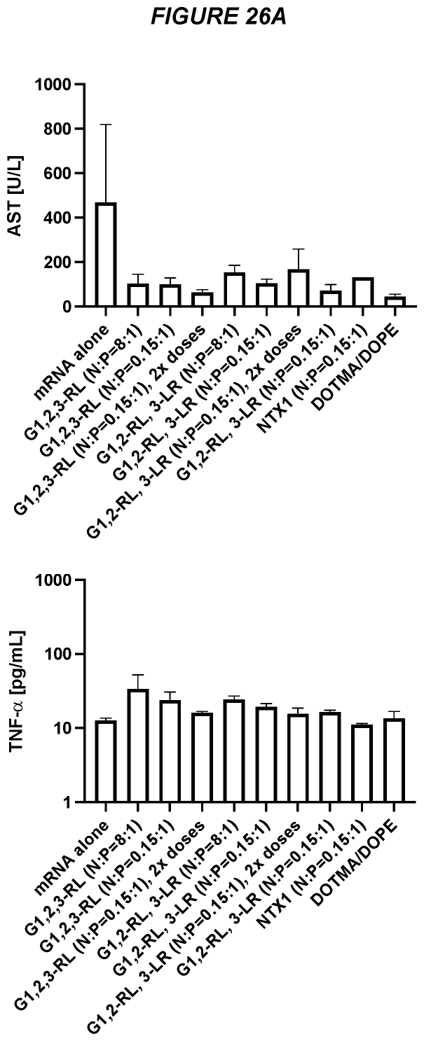

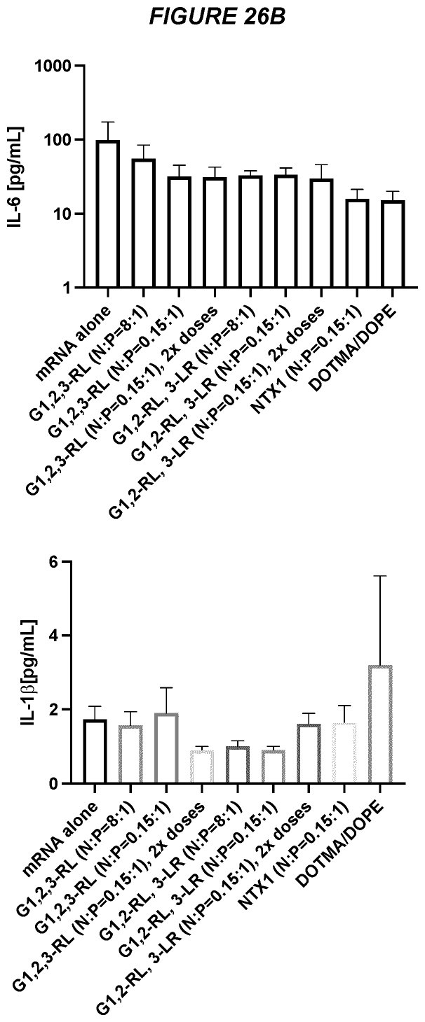

Aspartate transaminase (AST) Is a biomarker that can be used as a measure of liver health and can also be used as a marker to determine liver toxicity of an agent. All tested dendrimer-lipid-mRNA compositions tested showed no significant increase in AST levels compared to compositions comprising DOTMA/DOPE alone and in fact showed a decreased AST level compared to mRNA alone (FIG. 26A, top panel). These results indicate that the tested dendrimer compositions do not display any significant liver toxicity.

TNF-α, IL-6 and IL-1β were also measured in mice which received an injection of various dendrimer-lipid-mRNA formulations. As shown in FIG. 26A, bottom panel, and FIGS. 26B, top and bottom panels, none of the tested dendrimer compositions significantly increased plasma levels of TNF-α, IL-6 or IL-1β, respectively, compared to mRNA alone or DOTMA/DOPE and mRNA compositions. These data demonstrate that the dendrimer compositions do not cause a significant immune reaction in mice exposed to said compositions. This further demonstrates the safety of dendrimer compositions for use in delivering mRNA to tissues in vivo.

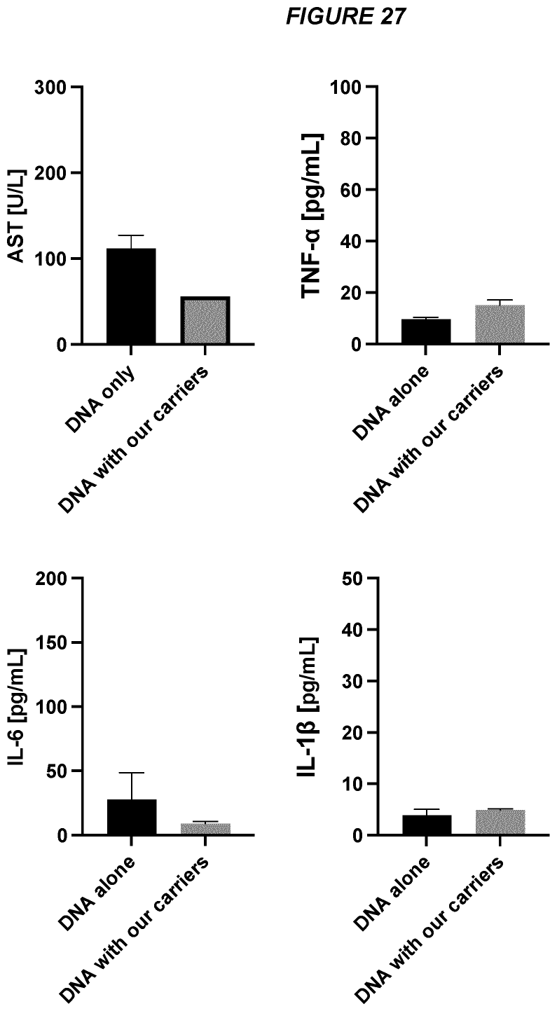

Mice were also injected with either DNA alone or a formulation comprising G1,2,3-RL, DNA and DOTMA/DOPE compositions and the plasma levels of AST, TNF-α, IL-6 and IL-13 measured after 24 hours. As shown in FIG. 27, injection with formulations comprising dendrimer did not cause a significant increase in the plasma levels of either AST (top left), TNF-α (top right), IL-6 (bottom left) or IL-1β (bottom right). These data indicate that delivery of DNA using the present dendrimers are safe and do not cause liver damage or an adverse immune reaction during.

Comparison of Peptide Dendrimer Transfection Efficiencies

The effect on the number of generations of dendrimers on mRNA delivery was studied by transfecting HeLa cells in full growth medium conditions. We have shown that dendrimers with 1 or 2 generations (e.g. RHCG1-R, RHCG1,2-R, RHCG1-LR, RHCG1-RL, 2-LR) can transfect cells effectively and mediate similar transfection efficiency as dendrimers with 3 generations (FIG. 28). This is indeed very different to DNA transfection because dendrimers with 1 or 2 generations do not transfect cells as well as dendrimers with 3 generations in full growth medium (FIG. 4). Interestingly, increasing the N:P ratios of the generation 1 or generation 2 dendrimers from 0.16 to 8 further increases the transfection efficiency (FIG. 29).

We have studied the effect of the core sequences of the dendrimers on transfection. We have changed the 3 amino acid core sequence, GSC, from G1,2-RL, 3-LR to only two amino acids, such as KA and YM. This change does not affect mRNA transfection, suggesting that dendrimers with 2 amino acids in the core would transfect as well as dendrimers with 3 amino acids in the core. Next, we substitute the 3 amino acid core sequences to different amino acids such as RFW, RYM, as compared to GSC. Although an arginine, which contains a cationic group, is added to the core, it does not improve or decrease transfection. Interestingly, when the 3 amino acids are changed to RHC from GCS at the core, transfection improves by 50%. This suggests that an ionisable group such as histidine in the core of the dendrimers can improve transfection (FIG. 28A).

We introduce 12 amino acids in the core of the dendrimer (LinearG1,2-RL, 3-LR), and transfection efficiency was not affected (FIG. 28A). This result indicates that it is possible to introduce a longer amino acid chain and/or a longer structure in the core without affecting transfection efficiency. Indeed, NTX1, which contains 27 amino acids in the core, transfection more effectively than G1,2-RL, 3-RL. This demonstrates that a longer core sequence can indeed enhance transfection. It is likely that the transfection enhancement from NTX1 is due to the presence of the cell penetrating peptide within the core sequence.

We also explore the effect on the number of amino acid within the generations on transfection. Thus we tested dendrimers with only 1 amino acid (R), 2 amino acids (RL or LR), 3 amino acids (RLR) and 4 amino acids (LRLR). Our transfection study indicates that dendrimers with 1, 2, 3 or 4 amino acids in each generation can still transfection mRNA well into cells (FIG. 28A). We even tested dendrimers with different number of amino acids in each generation and showed that transfection efficiency is not affected.

Based on the G1,2-RL, 3-LR structure, we have designed a library of 3 generation dendrimers in which we have replaced the basic amino acid R to K, and/or changing the hydrophobic amino acid L to an acidic amino acid such as E, and/or an amino acid with a non-polar side chain such as M, F, beta-alanine (B), aminohexanoic acid (X) and W and/or an amino acid with a polar side chain such as Q, T and Y.

Replacing R to K within the dendrimers reduces the mRNA transfection efficiency (FIG. 28A). However, changing the hydrophobic amino acids to other hydrophobic amino acids and/or amino acids with polar or non-polar side chains may not affect transfection or may reduce transfection by 30-40%, as compared to G1,2-RL, 3-LR. The underlining pattern is not clear and further investigation will be performed. However, al these dendrimers can induce mRNA transfection significantly higher than the mRNA alone control.

We have also investigated the impact of L- or D-form amino acids within the dendrimers on mRNA transfection. Based on the G1,2-RL, 3-LR dendrimer, we find that changing part or all the amino acids from L to D-form in each generation of the dendrimers would not affect transfection efficiency. Substituting the lysine to the diaminobutyric acid within dendrimer would reduce transfection, although the transfection of this dendrimer is still significantly higher than the mRNA alone control.

We have demonstrated that G1-LL, 2-RR can be used to deliver mRNA with our formulation protocol, in which we used G1-LL, 2-RR at a 0.16:1 N:P ratio with DOTMA/DOPE (w/w 10:1). Interestingly, this dendrimer was used to deliver ASO in vitro and in vivo in a different formulation (Saher 2018). The formulation used was DOTMA/DOPE (w/w=2:1 to ASO) and N:P=20:1, G1-LL, 2-RR to ASO. We have tried this on transfecting cells (i.e DOTMA/DOPE (w/w=2:1 to mRNA) and N:P=20:1, G1-LL, 2-RR to mRNA) and the transfection efficiency is poor. This formulation (DOTMA/DOPE (w/w=2:1 to mRNA) and N:P=20:1, G1-LL, 2-RR to mRNA) only yields 10% of the mRNA transfection efficiency of our improved formulation (DOTMA/DOPE (w/w=10:1 to mRNA) and N:P=0.16:1, G1-LL, 2-RR to mRNA).

We have explored the 3 generation dendrimers with either RL or LR or rl in different generations for mRNA delivery. We found that most of these dendrimers transfect cells similarly, with G1,2-RL, 3-LR being the most effective in mRNA transfection. Overall, our data suggested that dendrimer with hydrophobic and cationic amino acid in each generation would yield effective mRNA delivery to the cells.

Since there are 1 and 2 generation dendrimers transfecting cells more effective than G1,2-RL, 3-LR, we have selected these dendrimers for further testing on transfection. We showed that these dendrimers can in general transfect better than G1,2-RL, 3-LR in various N:P ratios. In particular, RHCG1-R, RHCG1,2-R, RHCG1-LR, RHCG1-RL, 2-LR, RHCG1-RLR and G1-LRLR at an N:P=4:1 can transfect 700% to 1815% better than G1,2-RL, 3-LR. These data demonstrated that the requirement for DNA and mRNA delivery is very different. For mRNA, there is a trend in which dendrimer with 1 or 2 generation would transfect cells better than 3 generation dendrimers in full growth medium conditions. However, generation 3 dendrimer would transfection cells with DNA way better than generation 1 or 2 dendrimers in full growth medium conditions (FIG. 4).

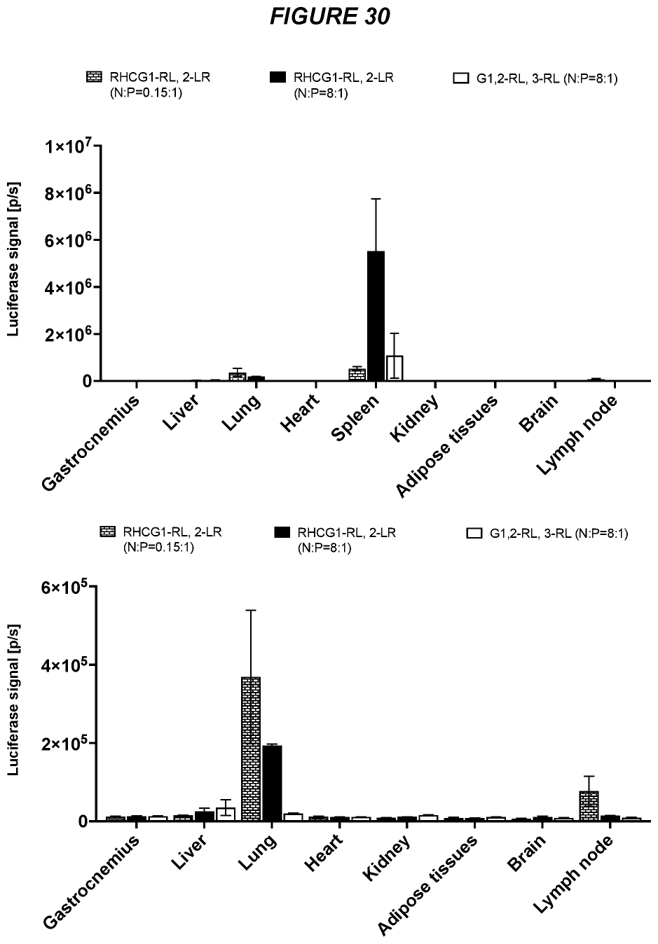

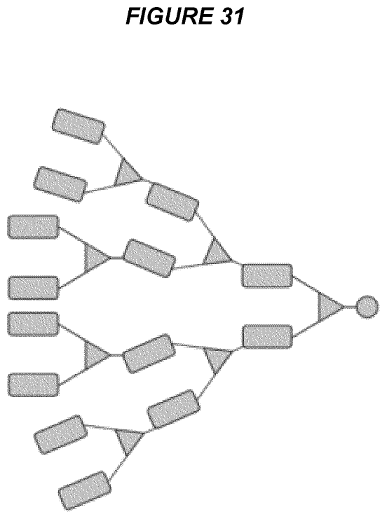

FIG. 30 shows that generation 2 dendrimers comprising an RHC peptide core delivered at an NP ratio of 8:1 targets mRNA delivery mainly to the spleen (top panel) with and to a lower extent the lung (bottom panel). At an NP ratio of 0.15:1, mRNA delivery to the lung is increased relative to an NP ratio of 8:1.

Modulating mRNA Expression

mRNA expression can be modulated by injecting mice with composition comprising PEG2000 lipid. Mice injected with a composition comprising G1,2-RL,3-LR, DOTMA/DOPE/PEG2000 and mRNA had attenuated luciferase expression compared to mice injected with a composition comprising G1,2-RL,3-LR, DOTMA/DOPE and mRNA.

Numbered Paragraphs

The following numbered paragraphs set out particular features and combinations of features of the present invention.
1. A composition for use in medicine, wherein the composition comprises a peptide dendrimer, a nucleic acid and a lipid, wherein the peptide dendrimer comprises: a first lysine residue and two first peptide motifs; two second lysine residues and four second peptide motifs; four third lysine residues and eight third peptide motifs; and a core peptide sequence which is covalently bound to the first lysine residue,
(i) wherein the first lysine residue is covalently bound to the two first peptide motifs, which are respectively covalently bound to the two second lysine residues;
(ii) wherein each second lysine residue is covalently bound to two second peptide motifs, wherein each second peptide motif is respectively covalently bound to one of the third lysine residues; and
(iii) wherein each third lysine residue is covalently bound to two of the third peptide motifs, wherein the first, second and third peptide motifs independently consist of monopeptide or dipeptide motifs, and wherein each of the first, second and third peptide motifs comprise a) an arginine (R) or lysine (K); and/or b) a leucine (L), valine (V), histidine (H) or isoleucine (I), wherein each amino acid residue is independently selected from the L-isoform or D-isoform.
2. The composition for the use according to paragraph 1, wherein at least two of the first, second and third peptide motifs comprise an arginine (R).
3. The composition for the use according to paragraph 1 or paragraph 2, wherein at least two of the first, second and third peptide motifs comprise a leucine (L).
4. The composition for the use according to any one of the preceding paragraphs, wherein each of the first, second and third peptide motifs are dipeptide motifs.
5. The composition for the use according to paragraph 4, wherein each of the first, second and third peptide motifs comprises a leucine (L) and an arginine (R).
6. The composition for the use according to any one of the preceding paragraphs, wherein the nucleic acid comprises a double stranded region.
7. The composition for the use according to paragraph 6, wherein the nucleic acid is a DNA plasmid.
8. The composition for the use according to any one of the preceding paragraphs, wherein the nucleic acid is, or encodes, an mRNA molecule or an antisense oligonucleotide (ASO).
9. The composition for the use according to any one of the preceding paragraphs, wherein the nucleic acid comprises a CRISPR sequence.
10. The composition for the use according to paragraph 6, wherein the nucleic acid is an siRNA or an saRNA molecule.
11. The composition for the use according to paragraph 7, wherein the DNA plasmid can express an siRNA or an saRNA molecule in a target cell.
12. The composition for the use according to paragraph 7 or paragraph 8, wherein the nucleic acid can express a transgene in a target cell.
13. The composition for the use according to paragraph 12, wherein the transgene is a viral protein, a bacterial protein or a protein of a microorganism that is parasitic to a mammal.
14. The composition for the use according to paragraph 13, for use as a vaccine.
15. The composition for the use according to paragraph 12, for use in a gene therapy for a genetic disorder in a patient.
16. The composition for the use according to paragraph 15, wherein the genetic disorder causes a muscular dystrophy in the patient.
17. The composition for the use according to paragraph 15, wherein the genetic disorder causes a myopathy in the patient.
18. The composition according to paragraph 12, for use in a method of treating diabetic limb ischemia, wherein the transgene is hepatocyte growth factor (HGF), vascular endothelial growth factor (VEGF) and/or Fibroblast growth factors (FGF).
19. The composition for the use according to any one of the preceding paragraphs, wherein the core peptide sequence consists of a tripeptide motif.
20. The composition according to paragraph 18, wherein the tripeptide motif comprises a glycine (G), a serine (S), and a cysteine (C) and/or an alanine (A).
19. The composition for the use according to any one of the preceding paragraphs, wherein the lipid comprises dioleoylphosphatidylethanolamine (DOPE) and/or N-[1-(2,3-dioleyloxy)propyl]-N,N,N-trimethylammonium chloride (DOTMA).
20. The composition for the use according to any one of the preceding paragraphs, wherein the lipid comprises dioleoylphosphatidylethanolamine (DOPE) and dioleoylphosphatidylglycerol (DOPG).
21. The composition for the use according to any one of the preceding paragraphs, wherein the use comprises delivery of the nucleic acid to the muscle of the patient.
22. A pharmaceutical composition comprising the composition according to any one of the preceding paragraphs, and a pharmaceutically acceptable excipient.
23. A method of delivering a nucleic acid into a target cell, the method comprising contacting the target cell with the composition according to any one of paragraphs 1 to 20, wherein the target cell is a myocyte, a hepatocyte, a stellate cell, a neurons, an astrocyte, a splenocyte, a lung cell, a cardiomyocyte, a kidney cell, an adipose cell or a tumour cell.
24. A peptide dendrimer comprising: a first lysine residue and two first peptide motifs; two second lysine residues and four second peptide motifs; four third lysine residues and eight third peptide motifs; and a core peptide sequence which is covalently bound to the first lysine residue,
(i) wherein the first lysine residue is covalently bound to the two first peptide motifs, which are respectively covalently bound to the two second lysine residues;
(ii) wherein each second lysine residue is covalently bound to two second peptide motifs, wherein each second peptide motif is respectively covalently bound to one of the third lysine residues; and
(iii) wherein each third lysine residue is covalently bound to two of the third peptide motifs,
wherein the first, second and third peptide motifs each consist of either (i) a leucine-arginine (LR) dipeptide motif, or (II) an arginine-leucine (RL) motif; wherein at least one of the first, second and third peptide motifs is (i) leucine-arginine (LR), wherein each amino acid residue is independently selected from the L-isoform or D-isoform.
25. A composition comprising a nucleic acid, a lipid and the peptide dendrimer according to paragraph 24.
26. The composition according to paragraph 25 for use in medicine.
27. Use of the composition according to paragraph 25 for delivering the nucleic acid into a cell, in vitro or ex vivo.
28. The composition or peptide dendrimer according to any one of paragraphs 1-22 or 24-26, for use in the treatment of Pompe disease, a muscle wasting disease, or a muscular dystrophy, e.g. Duchenne muscular dystrophy.

### REFERENCES

A number of publications are cited above in order to more fully describe and disclose the invention and the state of the art to which the invention pertains. Full citations for these references are provided below. The entirety of each of these references is incorporated herein.
Kwok et al. Comparative structural and functional studies of nanoparticle formulations for DNA and siRNA delivery; Nanomedicine: Nanotechnology, Biology and Medicine 7; 210-219 (2011).
Kwok et al. Peptide Dendrimer/Lipid Hybrid Systems Are Efficient DNA Transfection Reagents: Structure-Activity Relationships Highlight the Role of Charge Distribution Across Dendrimer Generations; ACSNano 7; 5 4668-4682 (2013).
Kwok et al. Systematic Comparisons of Formulations of Linear Oligolysine Peptides with siRNA and Plasmid DNA; Chem Biol Drug Des 87: 747-763 (2016).
Kwok at al. Developing small activating RNA as a therapeutic: current challenges and promises Therapeutic delivery 10(3):151-164 (2019)
Saher et al. Novel peptide-dendrimer/lipid/oligonucleotide ternary complexes for efficient cellular uptake and improved splice-switching activity; Eur J Pharmaceutics and Biopharmaceutics 132: 29-40 (2018).
Saher et a. Sugar and Polymer Excipients Enhance Uptake and Splice-Switching Activity of Peptide-Dendrimer/Lipid/Oligonucleotide Formulations. Pharmaceutics. 11(12): 666 (2019)
Sheridan et al. Gene therapy finds its niche. Nat Biotechnol 29 (2), 121-8 (2011).
Braum. Non-viral Vector for Muscle-Mediated Gene Therapy; Chapter 9 Muscle Gene Therapy, Springer Nature Switzerland AG D. Duan, J. R. Mendell (eds.) 157-178 (2019).
Ren at al. Structural basis of DOTMA for its high intravenous transfection activity in mouse; Gene Therapy 7, 764-768 (2000).
John et al. Human MicroRNA Targets; PLoS Biology, 11(2), 1862-1879 (2004).
Myers et al. Recombinant Dicer efficiently converts large dsRNAs into siRNAs suitable for gene silencing; Nature Biotechnology 21:324-328 (2003).
Lim at al. Engineered Nanodelivery Systems to Improve DNA Vaccine Technologies; Pharmaceutics 12(1) 30 (2020).
Luo et al. Arginine functionalized peptide dendrimers as potential gene delivery vehicles. Biomaterials 33, 4917-4927 (2012).
Bonnet at al. Systemic Delivery of DNA or siRNA Mediated by Linear Polyethylenimine (L-PEI) Does Not Induce an Inflammatory Response. Pharmaceutical Res. 25, 2972 (2008).
Wang at al. Adeno-associated virus vector as a platform for gene therapy delivery. Nat Rev Drug Discov. 18(5): 358-378 (2019).
Philippidis. Fourth Boy Dies in Clinical Trial of Astellas' AT132. Human Gene Therapy. 32, 19-20 (2021).
Qiu at al. Developing Biodegradable Lipid Nanoparticles for Intracellular mRNA Delivery and Genome Editing. Acc. Chem. Res. 54(21), 4001-4011 (2021).
Benizri at al. Bioconjugated Oligonucleotides: Recent Developments and Therapeutic Applications. Bioconjug Chem. 30(2): 366-383. (2019).
Jasinski at al. The Effect of Size and Shape of RNA Nanoparticles on Biodistribution. Mol Ther. 26(3), 784-792 (2018).
Huang et al. Delivery of Therapeutics Targeting the mRNA-Binding Protein HuR Using 3DNA Nanocarriers Suppresses Ovarian Tumor Growth. Cancer Research. 76(6), 1549-1559 (2016).
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A composition comprising a peptide dendrimer, a nucleic acid and a lipid, for use in medicine
wherein the peptide dendrimer comprises at least: a core peptide sequence, a first branching residue and two first peptide motifs, wherein the branching residue is lysine, 2,4-diaminobutyric acid, omithine, or diaminopropionic acid
and wherein the nucleic acid comprises a nucleic acid of at least 30, at least 35, at least 40, at least 45, or at least 50 nucleotides.

2. The composition for use according to claim 1, wherein the nucleic acid is RNA.

3. The composition for use according to claim 2, wherein the RNA is selected from an mRNA, an ssRNA, a dsRNA, an sgRNA, a crRNA, a tracrRNA, a lncRNA, an siRNA, an saRNA and/or a self-amplifying RNA.

4. The composition for use according to claim 3, wherein the RNA is mRNA.

5. The composition for use according to claim 1, wherein the nucleic acid is DNA.

6. The composition for use according to claim 5, wherein the DNA comprises a ssDNA, a dsDNA, a plasmid, and/or a cDNA.

7. The composition for use according to any one of the preceding claims, wherein the composition comprises an RNA nucleic acid and a DNA nucleic acid.

8. The composition for use according to claim 7, wherein the RNA nucleic acid and a DNA nucleic acid are part of a single nucleic acid molecule.

9. The composition for use according to any one of the preceding claims, wherein the nucleic acid comprises a modified nucleic acid.

10. The composition for use according to any one of the preceding claims, wherein the nucleic acid encodes a transgene and can express the transgene in a target cell.

11. The composition for use according to any one of the preceding claims, wherein the use involves treatment of a disease in the subject by modulating expression or activity of an endogenous gene.

12. The composition for use according to claim 11, wherein the modulation is an increase in the expression of the gene and/or exogenous expression of further copies of the gene.

13. The composition for use according to claim 11, wherein the modulation is a decrease in the expression of the gene.

14. The composition for use according to any one of claims 11-13, wherein the endogenous gene is translated to a protein or peptide.

15. The composition for use according to claim 14, wherein the protein or peptide comprises an antigen, a hormone, a receptor, a chimeric antigen receptor, a transcription factor and/or a cytokine such as IL-2, IL-7, IL-12, IL-15, IL-21 and/or interferon.

16. The composition for use according to claim 10, wherein the transgene comprises a tumour antigen, a viral protein, a bacterial protein or a protein of a microorganism that is parasitic to a mammal.

17. The composition for use according to any one of the preceding claims, wherein the composition is a vaccine.

18. The composition for use according to claim 17, wherein the nucleic acid comprises or encodes a self-amplifying RNA.

19. The composition for use according to any one of claims 1 to 16, wherein the use comprises a treatment for a genetic disorder in the subject.

20. The composition for use according to claim 19, wherein the nucleic acid expresses a functional version of a gene that is non-functional, downregulated, inactive or impaired in the subject.

21. The composition for use according to any one of the preceding claims, wherein the nucleic acid encodes and/or comprises one or more components of a system for editing a genome or a system for altering gene expression.

22. The composition for use according to claim 21, wherein the system for editing a genome or a system for altering gene expression is a CRISPR/Cas system.

23. The composition for use according to claim 21 or 22, wherein the nucleic acid encodes a Cas protein or peptide, and/or comprises an sgRNA, a crRNA, and/or a tracrRNA.

24. The composition for use according to claim 23, wherein the nucleic acid comprises an mRNA encoding a Cas protein or peptide, and an RNA sequence comprising sgRNA.

25. The composition for use according to claim 24, wherein the mRNA encoding a Cas protein or peptide, and the RNA sequence comprising sgRNA are separate molecules.

26. The composition for use according to claim 23 or 24, wherein one or more of the sgRNA, crRNA, tracrRNA and nucleic acid encoding a Cas protein, where present, are part of a single nucleic acid.

27. The composition for use according to claim 23 or 24, wherein one or more of the sgRNA, crRNA, tracrRNA and nucleic acid encoding a Cas protein, where present, are present on two or more nucleic acids.

28. The composition for use according to any one of the preceding claims, wherein the composition is targeted to spleen, lymph tissue, lung, bone, thymus, liver, tumour tissue, cardiac tissue, skeletal muscle, kidney, adipose tissues and/or brain.

29. The composition for use according to claim 28, wherein the composition is targeted to spleen, lymph tissue, lung and/or bone.

30. The composition for use according to claim 29, wherein the nucleic acid is RNA, e.g. mRNA.

31. The composition for use according to any one of the preceding claims, wherein the use comprises administering the composition to a subject such that the nucleic acid is delivered to a cell that is a leucocyte, e.g. a B lymphocyte, a T lymphocyte, a monocyte, a neutrophil, a dendritic cell, a macrophage, or a monocyte; a lymph node tissue cell, a myeloid cell, a fibroblast, a myocyte, a skeletal myocyte, an endothelial cell, a hepatocyte, a stellate cell, a neuron, an astrocyte, a splenocyte, a lung cell, a cardiomyocyte, a kidney cell, an adipose cell, a stem cell and/or a tumour cell.

32. The composition for use according to any one of the preceding claims, wherein the use comprises administering the composition to a subject such that the nucleic acid is delivered to an immune cell.

33. The composition for use according to claim 31 or 32, wherein the nucleic acid expresses an immune molecule or a transcription factor in the cell.

34. The composition for use according to claim 33, wherein the immune molecule is a T cell receptor, chimeric antigen receptor, a cytokine, a decoy receptor, an antibody, a costimulatory receptor, a costimulatory ligand, a checkpoint inhibitor, an immunoconjugate, or a tumour antigen.

35. The composition for use according to any one of claims 31 to 34, wherein the cell is a B lymphocyte, a T lymphocyte, a neutrophil, a dendritic cell, a macrophage, a monocyte, a myeloid derived suppressor cell (MDSC), a tumour associated macrophage or a tumour associated neutrophil.

36. The composition for use according to claim 35, wherein the nucleic acid is RNA, e.g. mRNA.

37. The composition for use according to any one of the preceding claims, wherein the composition is for use in a method of treating cancer in the subject.

38. The composition for use according to claim 37, wherein the cancer is a blood cancer, for example leukaemia, lymphoma, myeloma, myelodysplastic syndrome; or a lung cancer, a cardiac cancer, a sarcoma, or a liver cancer.

39. The composition for use according to claim 37 or claim 38, wherein the method comprises administration of an anticancer agent.

40. The composition for use according to any one of the preceding claims, wherein the composition is for use in a method of treating a lung disease or an autoimmune disease in the subject.

41. The composition for use according to any one of claims 19 to 28, wherein the method is a treatment for Pompe disease, a muscle wasting disease, a myopathy, or a muscular dystrophy, e.g. Duchenne muscular dystrophy in the subject.

42. The composition for use according to claim 41, wherein the nucleic acid is DNA.

43. The composition for use according to any one of claims 1 to 31, wherein the method is a treatment for a limb ischemia, such as diabetic limb ischemia, in the subject, and wherein the transgene is hepatocyte growth factor (HGF), vascular endothelial growth factor (VEGF) and/or Fibroblast growth factor (FGF).

44. The composition for use according to any one of the preceding claims, wherein the two first peptide motifs independently consist of a single amino acid, dipeptide, tripeptide or tetrapeptide motifs.

45. The composition for use according to any one of the preceding claims, wherein the peptide dendrimer further comprises two second branching residues (e.g. lysine) and four second peptide motifs,
wherein one of the second branching residues is covalently bound to one of the first peptide motifs and the other second branching residue is covalently bound to the other first peptide motif, and wherein each second branching residue is covalently bound to two second peptide motifs.

46. The composition for use according to claim 45, wherein the four second peptide motifs independently consist of a single amino acid, dipeptide, tripeptide or tetrapeptide motifs.

47. The composition for use according to claim 45 or 46, wherein the peptide dendrimer further comprises four third branching residues (e.g. lysine) and eight third peptide motifs,
wherein each second peptide motif is respectively covalently bound to one of the third branching residues such that each third branching residue is covalently bound to one second peptide motif, and wherein each third branching residue is covalently bound to two third peptide motifs.

48. The composition for use according to claim 47, wherein the eight third peptide motifs independently consist of a single amino acid, dipeptide, tripeptide or tetrapeptide motifs.

49. The composition for use according to any one of claims 44 to 48, wherein the first, second and/or third peptide motifs comprise an amino acid with a basic side chain.

50. The composition for use according to any one of claims 44 to 49, wherein the first, second and/or third peptide motifs comprise an amino acid with a non-polar side chain.

51. The composition for use according to any one of claims 44 to 50, wherein the first, second and/or third peptide motifs comprise an amino acid with an acidic side chain.

52. The composition for use according to any one of claims 44 to 51, wherein the first, second and/or third peptide motifs comprise an amino acid with an uncharged polar side chain.

53. The composition for use according to any one of claims 44 to 52, wherein the first, second and/or third peptide motifs comprise a leucine (L) and/or arginine (R) residue.

54. The composition for use according to any one of claims 44 to 53, wherein the core sequence comprises at least two amino acids.

55. The composition for use according to any one of claims 44 to 54, wherein the core sequence comprises up to 30 amino acids.

56. The composition for use according to any one of claims 44 to 54, wherein the core sequence comprises an ionisable amino acid such as histidine.

57. The composition for use according to any one of the preceding claims, wherein the peptide dendrimer comprises a structure set forth in Table 2.

58. The composition for use according to any one of the preceding claims, wherein the peptide dendrimer further comprises a tissue and/or cell targeting motif.

59. The composition for use according to claim 58, wherein the tissue or cell targeting motif comprises a muscle targeting motif, for example, GAASSLNIA (SEQ ID NO: 1), an integrin targeting motif, for example arginine-glycine-aspartic acid or a chemical modification, for example comprising mannose glycosylation.

60. The composition for use according to any one of the preceding claims, wherein the peptide dendrimer further comprises a cell penetrating peptide (CPP).

61. The composition for use according to claim 60, wherein the cell penetrating peptide comprises the peptide sequence XRXRRBRRXRRBRXB (SEQ ID NO: 2), where X is 6-aminohexanoic acid and B is beta-alanine.

62. The composition for use according to claim 60, wherein the cell penetrating peptide comprises a TAT derived sequence.

63. The composition for use according to any one of the preceding claims, wherein the peptide dendrimer further comprises an alkyl chain, alkenyl chain, an antibody or a fragment thereof, a targeting peptide, a sugar, a cell penetrating peptide, an endosomal escape peptide, a nuclear localisation motif, and/or a fatty acid.

64. The composition for use according to claim 63, wherein the alkyl or alkenyl chain is conjugated to the core peptide sequence.

65. The composition for use according to claim 63 or 64, wherein the alkyl or alkenyl chain, antibody or fragment thereof, the targeting peptide, sugar, targeting peptide, cell penetrating peptide, endosomal escape peptide, nuclear localisation motif, and/or fatty acid is conjugated to the C terminus of the peptide dendrimer.

66. The composition for use according to any one of claims 63 to 65, wherein the alkyl or alkenyl chain, antibody or the fragment thereof, targeting peptide, sugar, targeting peptide, cell penetrating peptide, endosomal escape peptide, nuclear localisation motif, and/or fatty acid is conjugated to the N terminus of the peptide dendrimer.

67. The composition for use according to any one of claims 63 to 66, wherein the alkyl or alkenyl chain comprises from about 5 carbons to about 50 carbons, preferably from about 12 to about 30 carbons.

68. The composition for use according to any one of the preceding claims, wherein the lipid comprises a cationic lipid, a neutral lipid, an anionic lipid and/or an ionisable lipid.

69. The composition for use according to any one of the preceding claims, wherein the lipid comprises a saturated fatty acid.

70. The composition for use according to any one of the preceding claims, wherein the lipid comprises an unsaturated fatty acid.

71. The composition for use according to claim 69 or 70, wherein the lipid comprises 1, 2, 3, 4, 5 or 6 fatty acid chains.

72. The composition for use according to any one of the preceding claims, wherein the lipid comprises dioleoylphosphatidylethanolamine (DOPE) and/or N-[1-(2,3-dioleyloxy)propyl]-N,N,N-trimethylammonium chloride (DOTMA).

73. The composition for use according to any one of the preceding claims, wherein the lipid comprises dioleoylphosphatidylethanolamine (DOPE) and dioleoylphosphatidylglycerol (DOPG).

74. The composition for use according to any one of the preceding claims, wherein the N/P ratio is between about 0.01:1 and 100:1.

75. The composition for use according to claim 74, wherein the N/P ratio is between 1:1 and 50:1.

76. The composition for use according claim 75, wherein a higher proportion of the composition is observed in the spleen and/or lymph nodes than the liver following administration to a subject.

77. The composition for use according claim 75, wherein the N/P ratio is between 0.01:1 and 1:1

78. The composition for use according to claim 77, wherein a higher proportion of the composition is observed in the lung, spleen and/or lymph nodes than the liver following administration to a subject.

79. The composition for use according to any one of the preceding claims, wherein the peptide dendrimer, nucleic acid and lipid form a positively charged particle.

80. The composition for use according to any one of the preceding claims, wherein the peptide dendrimer, nucleic acid and lipid form a negatively charged particle or a particle with neutral charge.

81. The composition for use according to any one of claims 28 to 32, wherein delivery of the nucleic acid to the target tissue or cell is increased by at least 10%, 15%, 20%, 25%, 30%, 35%, 40%, 45%, 50%, 60%, 65%, 75%, 85%, 90%, 95%, 100% compared to delivery of the same nucleic acid to the same tissue or cell type using a lipid based nucleic acid delivery system.

82. The composition for use according to claim 81, wherein the target tissue is spleen, lymph node, lung, liver or bone.

83. The composition for use according to claim 81 or 82, wherein the lipid based nucleic acid delivery system is DOTMA/DOPE.

84. The composition for use according to any one of the preceding claims, wherein the administration is intravenous, intramuscular, intratumoral, subcutaneous, intradermal or intraperitoneal.

85. The composition for use according to any one of the preceding claims, wherein the composition is comprised within a liquid.

86. A composition comprising a peptide dendrimer, a nucleic acid and a lipid, wherein the peptide dendrimer comprises at least: a core peptide sequence, a first branching residue and two first peptide motifs wherein the branching residue is lysine, 2,4-diaminobutyric acid, omithine, or diaminopropionic acid;
wherein the nucleic acid comprises a single-stranded nucleic acid of at least 30, at least 35, at least 40, at least 45, or at least 50 nucleotides.

87. The composition according to claim 86, wherein the single-stranded nucleic acid is RNA.

88. The composition according to claim 87, wherein the RNA is selected from an mRNA, an ssRNA, an sgRNA, a crRNA, a tracrRNA, a lncRNA, and/or a self-amplifying RNA.

89. The composition according to claim 88, wherein the RNA is mRNA.

90. The composition according to any one of claims 86 to 89, wherein the composition comprises two or more RNAs.

91. The composition according to claim 86, wherein the single-stranded nucleic acid is a ssDNA.

92. The composition according to any one of claims 86 to 91, wherein the composition comprises an RNA nucleic acid and a DNA nucleic acid.

93. The composition according to claim 92, wherein the RNA nucleic acid and a DNA nucleic acid are part of a single nucleic acid molecule.

94. The composition according to any one of claims 86 to 93, wherein the single-stranded nucleic acid comprises a modified nucleic acid.

95. The composition according to any one of claims 86 to 94, wherein the single-stranded nucleic acid encodes a transgene and can express the transgene in a target cell.

96. The composition according to any one of claims 86 to 95, wherein the single-stranded nucleic acid can modulate expression or activity of an endogenous gene.

97. The composition according to claim 96, wherein the modulation is an increase in the expression of the gene and/or exogenous expression of further copies of the gene.

98. The composition according to claim 96, wherein the modulation is a decrease in the expression of the gene.

99. The composition according to any one of claims 96 to 98, wherein the endogenous gene is translated to a protein or peptide.

100. The composition according to claim 99, wherein the protein or peptide comprises an antigen, a hormone, a receptor, a chimeric antigen receptor, a transcription factor and/or a cytokine such as IL-2, IL-7, IL-12, IL-15, IL-21 and/or interferon.

101. The composition according to claim 95, wherein the transgene comprises a viral protein, a bacterial protein and/or a protein of a microorganism that is parasitic to a mammal.

102. The composition according to any one of claims 86 to 101, wherein the composition is vaccine.

103. The composition according to claim 108, wherein the single-stranded nucleic acid comprises or encodes a self-activating RNA.

104. The composition according to any one of claims 86 to 100, wherein the use comprises a treatment for a genetic disorder in the subject.

105. The composition according to claim 104, wherein the nucleic acid expresses a functional version of a gene that is non-functional, downregulated, inactive or impaired in the subject.

106. The composition according any one of claims 86 to 105, wherein the single-stranded nucleic acid encodes and/or comprises one or more components of a system for editing a genome or a system for altering gene expression.

107. The composition according to claim 106, wherein the system for editing a genome or a system for altering gene expression is a CRISPR/Cas system.

108. The composition according to claim 106 or 107, wherein the single-stranded nucleic acid encodes a Cas protein or peptide, and/or comprises an sgRNA, a crRNA, and/or a tracrRNA.

109. The composition according to claim 108, wherein the single-stranded nucleic acid comprises an mRNA encoding a Cas protein or peptide, and an sgRNA.

110. The composition according to claim 108 or 109, wherein one or more of the sgRNA, crRNA, tracrRNA and nucleic acid encoding a Cas protein, where present, are part of a single nucleic acid.

111. The composition according to claim 108 or 109, wherein one or more of the sgRNA, crRNA, tracrRNA and nucleic acid encoding a Cas protein, where present, are present on two or more nucleic acids.

112. The composition according to any one of claims 86 to 111, wherein the composition is suitable for targeting the nucleic acid to spleen, lymph tissue, lung, bone, liver, cardiac tissue, tumour tissue, skeletal muscle, kidney, adipose tissues and/or brain.

113. The composition according to claim 112, wherein the composition is suitable for targeting the nucleic acid to spleen, lymph tissue, lung and/or bone.

114. The composition according to claim 113, wherein the nucleic acid is RNA, e.g. mRNA.

115. The composition according to any one of the claims 86 to 114, for use in a method of treating cancer in a subject.

116. The composition for use according to claim 115, wherein the cancer is a blood cancer, for example leukaemia, lymphoma or myeloma, cardiac cancer, a sarcoma, or liver cancer.

117. The composition for use according to claim 115 or claim 116, wherein the method comprises administration of an anticancer agent.

118. The composition according to any one of claims 86 to 114, for use in a method of treating a lung disease or an autoimmune disease in a subject.

119. The composition according to any one of claims 86 to 114, for use in treating Pompe disease, a muscle wasting disease, a myopathy, or a muscular dystrophy, e.g. Duchenne muscular dystrophy in a subject.

120. The composition for use according to claim 119, wherein the nucleic acid is DNA.

121. The composition for use according to any claim 86 to 120, wherein the composition is delivers the nucleic acid to a leucocyte, e.g. a B lymphocyte, a T lymphocyte, a monocyte, a neutrophil, a dendritic cell, a macrophage, or a monocyte; a lymph node tissue cell, a myeloid cell, a fibroblast, a myocyte, a skeletal myocyte, a hepatocyte, a stellate cell, a neuron, an astrocyte, a splenocyte, a lung cell, a cardiomyocyte, a kidney cell, an adipose cell or a tumour cell in the subject.

122. The composition for use according to any one of claims 86 to 121, wherein the use comprises administering the composition to a subject such that the nucleic acid is delivered to an immune cell.

123. The composition for use according to claim 121 or 122, wherein the nucleic acid expresses an immune molecule or a transcription factor in the cell.

124. The composition for use according to claim 123, wherein the immune molecule is a T cell receptor, a cytokine, a decoy receptor, an antibody, a costimulatory receptor, a costimulatory ligand, a checkpoint inhibitor, an immunoconjugate, or a tumour antigen.

125. The composition for use according to any one of claims 121 to 124, wherein the cell is a B lymphocyte, a T lymphocyte, a monocyte, a neutrophil, a dendritic cell, a macrophage, or a monocyte.

126. The composition for use according to claim 125, wherein the nucleic acid is RNA, e.g. mRNA.

127. The composition according to any one of claims 86 to 97, or the composition for use according to any one of claims 115 to 126, wherein the two first peptide motifs independently consist of a single amino acid, dipeptide, tripeptide or tetrapeptide motifs.

128. The composition or the composition for use according to claim 127, wherein the peptide dendrimer further comprises two second branching residues (e.g. lysine) and four second peptide motifs,
wherein one of the second branching residues is covalently bound to one of the first peptide motifs and the other second branching residue is covalently bound to the other first peptide motif, and wherein each second branching residue is covalently bound to two second peptide motifs.

129. The composition or the composition for use according to claim 128, wherein the four second peptide motifs independently consist of monopeptide, dipeptide, tripeptide or tetrapeptide motifs.

130. The composition or the composition for use according to claim 127 or 128, wherein the peptide dendrimer further comprises four third branching residues (e.g. lysine) and eight third peptide motifs,
wherein each second peptide motif is respectively covalently bound to one of the third branching residues such that each third branching residue is covalently bound to one second peptide motif, and wherein each third branching residue is covalently bound to two third peptide motifs.

131. The composition or the composition for use according to claim 130, wherein the eight third peptide motifs independently consist of monopeptide, dipeptide, tripeptide or tetrapeptide motifs.

132. The composition or composition for use according to any one of claims 127 to 131, wherein the first, second and/or third peptide motifs comprise an amino acid with a basic side chain.

133. The composition or composition for use according to any one of claims 127 to 132, wherein the first, second and/or third peptide motifs comprise an amino acid with a non-polar side chain.

134. The composition or composition for use according to any one of claims 127 to 133, wherein the first, second and/or third peptide motifs comprise an amino acid with an acidic side chain.

135. The composition or composition for use according to any one of claims 127 to 134, wherein the first, second and/or third peptide motifs comprise an amino acid with an uncharged polar side chain.

136. The composition or composition for use according to any one of claims 127 to 135, wherein the first, second and/or third peptide motifs comprise a leucine (L) and/or arginine (R) residue.

137. The composition or composition for use according to any one of claims 86 to 136, wherein the peptide dendrimer comprises a structure set forth in Table 2.

138. The composition or the composition for use according to any one of claims 86 to 137, wherein the peptide dendrimer comprises a tissue and/or cell targeting motif.

139. The composition or the composition for use according to claim 138, wherein the tissue targeting motif comprises a muscle targeting motif, for example, GAASSLNIA (SEQ ID NO: 1), an integrin targeting motif, for example arginine-glycine-aspartic acid or a chemical modification, for example comprising mannose glycosylation.

140. The composition or the composition for use according to any one of claims 86 to 139, wherein the peptide dendrimer comprises a cell penetrating peptide.

141. The composition for use according to claim 140, wherein the cell penetrating peptide comprises a TAT derived sequence.

142. The composition or the composition for use according to claim 140, wherein the cell penetrating peptide comprises the peptide sequence XRXRRBRRXRRBRXB (SEQ ID NO: 2), where X is 6-aminohexanoic acid and B is beta-alanine.

143. The composition or the composition for use according to any one of claims 86 to 142, wherein the peptide dendrimer comprises an alkyl chain, alkenyl chain, an antibody or a fragment thereof, a sugar, and/or a fatty acid.

144. The composition or the composition for use according to claim 143, wherein the alkyl or alkenyl chain is conjugated to the core peptide sequence.

145. The composition for use according to claim 144, wherein the alkyl or alkenyl chain is conjugated to the C terminus of the peptide dendrimer.

146. The composition for use according to claim 144, wherein the alkyl or alkenyl chain is conjugated to the N terminus of the peptide dendrimer

147. The composition or the composition for use according to any one of claims 143 to 146, wherein the alkyl or alkenyl chain comprises from about 5 carbons to about 50 carbons, preferably from about 12 to about 30 carbons.

148. The composition or the composition for use according to claim 143, wherein the peptide dendrimer comprises a fatty acid conjugated to the C terminus of the peptide dendrimer.

149. The composition for use according to claim 143, wherein the peptide dendrimer comprises a fatty acid conjugated to the N terminus of the peptide dendrimer.

150. The composition for use according to any one of claims 86 to 149, wherein the lipid comprises a cationic lipid, a neutral lipid, an anionic lipid and/or an ionisable lipid.

151. The composition for use according to any one of claims 86 to 150, wherein the lipid comprises a saturated fatty acid.

152. The composition for use according to any one of claims 86 to 151, wherein the lipid comprises an unsaturated fatty acid.

153. The composition for use according to claim 151 or 152, wherein the lipid comprises 1, 2, 3, 4, 5 or 6 fatty acid chains.

154. The composition or the composition for use according to any one of claims 86 to 153, wherein the lipid comprises dioleoylphosphatidylethanolamine (DOPE) and/or N-[1-(2,3-dioleyloxy)propyl]-N,N,N-trimethylammonium chloride (DOTMA).

155. The composition or the composition for use according to any one of claims 86 to 154, wherein the lipid comprises dioleoylphosphatidylethanolamine (DOPE) and dioleoylphosphatidylglycerol (DOPG).

156. The composition or the composition for use according to any one of claims 86 to 155, wherein the N/P ratio is between about 0.01:1 and 100:1.

157. The composition or the composition for use according to claim 156, wherein the NIP ratio is between 1:1 and 50:1.

158. The composition or the composition for use according claim 157, wherein a higher proportion of the composition is observed in the spleen and/or lymph nodes than the liver following administration to a subject.

159. The composition or the composition for use according claim 156, wherein the N/P ratio is between 0.01:1 and 1:1

160. The composition or the composition for use according to claim 159, wherein a higher proportion of the composition is observed in the lung, spleen and/or lymph nodes than the liver following administration to a subject.

161. The composition for according to any one of claims 86 to 160, wherein following contacting a target cell with the composition in vitro, delivery of the nucleic acid to the target cell is increased by at least 10%, 15%, 20%, 25%, 30%, 35%, 40%, 45%, 50%, 60%, 65%, 75%, 85%, 90%, 95%, 100% compared to delivery of the same nucleic acid to the same cell type using a lipid based nucleic acid delivery system.

162. The composition for use according to claim 161, wherein the target cell is a cancer cell or an immune cell.

163. The composition for use according to claim 161 or 162, wherein the lipid based nucleic acid delivery system is Lipofectamine 2000.

164. The composition or the composition for use according to any one of claims 86 to 163, wherein the peptide dendrimer, nucleic acid and lipid form a positively charged particle.

165. The composition or the composition for use according to any one of claims 86 to 164, wherein the composition is comprised within a liquid.

166. The composition according to any one of claims 86 to 165, wherein the composition is a powder composition.

167. A method of delivering a nucleic acid into a cell in a subject in need of the delivery, comprising administering a pharmaceutically effective amount of a composition according to any one of claims 1 to 165 to the subject.


SEQUENCE LISTING (3 sequences)

SEQ ID NO: 1 - protein, 9 aa, Artificial Sequence
  Muscle targeting motif
GAASSLNIA

SEQ ID NO: 2 - protein, 15 aa, Artificial Sequence
  Cell penetrating peptide
  MOD_RES (1)..(1): Xaa= 6-aminohexanoic acid
  MOD_RES (3)..(3): Xaa= 6-aminohexanoic acid
  MOD_RES (6)..(6): Ala= beta-alanine
  MOD_RES (9)..(9): Xaa= 6-aminohexanoic acid
  MOD_RES (12)..(12): Ala= beta-alanine
  MOD_RES (14)..(14): Xaa= 6-aminohexanoic acid
  MOD_RES (15)..(15): Ala= beta-alanine
XRXRRARRXRRARXA

SEQ ID NO: 3 - protein, 7 aa, Artificial Sequence
  Muscle targeting peptide
ASSLINA